(19) United States
(12) Patent Application Publication
(10) Pub. No.: US 2011/0166088 A1
(43) Pub. Date: Jul. 7, 2011

(54) BENZOTHIAZOLES AND AZA-ANALOGUES THEREOF USE AS ANTIBACTERIAL AGENTS

(75) Inventors: Jitendra A. Sattigeri, Gurgaon (IN); Naresh Kumar, Gurgaon (IN); Ajay Yadav, Gurgaon (IN); Lalima Sharma, Chandigarh (IN); Ian A. Cliffe, Gurgaon (IN); Shibu B. Varughese, Ahmed Nagar (IN); Shaikh Rizwan Shabbir, New Delhi (IN); V. Samuel Raj, Sivakasi (IN); Dilip J. Upadhyay, Kalyan West (IN); Pradip K. Bhatnager, Exton, PA (US)

(73) Assignee: RANBAXY LABORATORIES LIMITED, New Delhi, Delhi (IN)

(21) Appl. No.: 13/000,561

(22) PCT Filed: Jun. 25, 2009
(86) PCT No.: PCT/IB09/52753

§ 371 Date: Mar. 28, 2011

(30) Foreign Application Priority Data
Jun. 25, 2008 (IN) 1525/DEL/2008

(51) Int. Cl. A61K 31/7068; C07D 417/14; C07D 413/14; C07H 17/02; A61K 31/444; A61K 31/4439; A61K 31/496; A61K 31/5355; A61K 31/513; A61K 31/506; A61P 31/04

(52) U.S. Cl. 514/27; 546/256; 546/270.1; 544/364; 544/131; 544/316; 544/333; 544/295; 536/17.4; 514/333; 514/338; 514/253.1; 514/233.8; 514/274; 514/256; 514/252.18

(57) ABSTRACT

The present invention provides Gyrase B and/or Topoisomerase IV par E inhibitors, which can be used as antibacterial agents. Compounds disclosed herein can be used for treating or preventing conditions caused by or contributed by gram positive, gram negative and anaerobic bacteria, more particularly against, for example, Staphylococci, Streptococci, Enterococci, Haemophilus, Pseudomonas spp., Acenetobacter spp., Moraxalla spp., Chlamydia spp., Mycoplasma spp., Legionella spp., Ktycobacterium spp., Helicobacter, Clostridium spp., Bacteroides spp., Corynebacterium, Bacillus spp., Enterobactericeae,’ (E. coli, Klebsiella spp., Proteus spp., etc.) or any combination thereof. Also provided, are processes for preparing compounds disclosed herein, pharmaceutical compositions containing compounds disclosed herein, and methods of treating bacterial infections. (Formula)

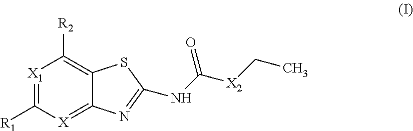

### FIELD OF INVENTION

The present invention provides Gyrase B and/or Topoisomerase IV par E inhibitors, which can be used as antibacterial agents. Compounds disclosed herein can be used for treating or preventing conditions caused by or contributed by gram positive, gram negative and anaerobic bacteria, more particularly against, for example, Staphylococci, Streptococci, Enterococci, Haemophilus, Pseudomonas spp., Acenetobacter spp., Moraxalla spp., Chlamydia spp., Mycoplasma spp., Legionella spp., Mycobacterium spp., Helicobacter, Clostridium spp., Bacteroides spp., Corynebacterium, Bacillus spp., Enterobactericeae (E. coli, Klebsiella spp or, Proteus spp.) or any combination thereof. Also provided, are processes for preparing compounds disclosed herein, pharmaceutical compositions containing compounds disclosed herein, and methods of treating bacterial infections.

### BACKGROUND OF THE INVENTION

Emergence of drug resistance to the existing drugs and increasing need of drugs to treat the endemic and epidemic diseases have driven major pharmaceutical companies to discover novel antibacterial targets. The international microbiological community continues to express serious concern that the evolution of bacterial resistance could result in strains against which currently available antibacterial agents will be ineffective. In general, bacterial pathogens may be classified either gram-positive or gram-negative pathogens. Antibiotics which are effective against both types of organisms are called as broad-spectrum antibiotics. Gram-positive organisms are particularly important for example, Staphylococci, Enterococci, Streptococci and Mycobacterium because of the development of resistant strain that are both difficult to treat and difficult to eradicate from the hospital environment once established.

Fluoroquinolones have been used to treat a great variety of infection including respiratory tract infections (Smith H. J. et al., “J. Antimicrobial Chemother.” 2002, 49, 893-895). As a result of their wide spectrum of activity, quinolones have been extensively used. Because of this high level use and to some degree of misuse, it has caused rapid development of bacterial resistance to these agents. With the approval of the three most recent antibacterial agents, linezolid in 2000, daptomycin in 2004 and telithromycin in 2002-04, three new classes of agents have been introduced into the market. However, resistance has already been reported for all these three agents, thus providing an opportunity for additional agents in these classes to overcome the new resistance identified. In addition, new targets should be explored to avoid these resistance already reported in the existing classes of antibiotics.

Methicillin resistant Staphylococcus aureus (MRSA) infections constitute the single most important cause of health care-associated infections, increasing lengths of hospital stay, severity of illness, deaths and costs. Although these infections occurred primarily in hospitals, they are becoming increasingly common in communities nationwide, especially where groups of people are in close quarters, including military facilities, sports teams and prisons. MRSA infection is more difficult to treat because the bacteria are resistant to β-lactam antibiotics such as methicillin, oxacillin, penicillin and amoxicillin They are also resistant to macrolides, fluoroquinolones, clindamycin and trimethoprim/sulfamethoxazole. These infections can progress to life-threatening blood or bone infections because there are fewer effective antibiotics available for treatment. The treatment for MRSA may be longer, more expensive and more complicated, and infections can reappear frequently. The glycopeptide antibiotics, teicoplanin and vancomycin are currently the mainstay of treatment of infections with MRSA. However, strains of MRSA have emerged to show intermediate susceptibility to glycopeptide antibiotics (GISA), or vancomycin (VISA). Oxazolidinones are new class of molecules active against MRSA and linezolid is the only drug available in the market. However, the toxicity of linezolid is the major issue and linezolid resistance has started emerging.

As a result, the need to combat drug-resistant bacteria and the increasing failure of the available drugs, there has been resurgent interest in discovering new antibiotics particularly those with either a novel mechanism of action and/or containing new pharmacophoric groups. One attractive strategy for the development of new antibiotics is to inhibit DNA gyrase, a bacterial enzyme necessary for DNA replication and therefore, necessary for bacterial cell growth and division. Gyrase activity is also associated with events in DNA transcription, repair and recombination.

DNA topoisomerases are enzymes that control the topology of the DNA in cells. DNA gyrase and topoisomerase IV are essential enzymes and play important role in DNA replication and compaction (Drlica and Zhao, “Microbiol Mol Biol Rev.” 1997, 61, 377-92). DNA supercoiling activity is essential in all bacteria but not found in humans and it is an ideal target for antibacterials. Gyrase catalyzes the conversion of relaxed, closed circular duplex DNA to a negatively superhelical form, which is more favorable for recombination. The mechanism of supercoiling reaction involves the wrapping of gyrase around a region of the DNA, double strand breaking in that region, passing a second region of the DNA through the break and rejoining the broken strands (Maxwell, A. “Trends Microbiol” 1997, 5, 102-109; Drlica and Zhao, “Microbiol Mol Biol Rev.” 1997, 61, 377-92). The supercoiling reaction is driven by the binding of ATP to gyrase and the ATP is then hydrolyzed during the reaction (Levine C. et al., “Biochim Biophys Acta” 1998, 1400, 29-43). This ATP binding and subsequent hydrolysis cause conformational changes in the DNA-bound gyrase that are necessary for its activity.

Bacterial DNA gyrase is a 400 kilodalton protein consisting of A₂B₂ heterotetramer (Maxwell, A. “Trends Microbiol” 1997, 5, 102-109). The A subunit (gyrA) comprises an N-terminal domain involved in DNA cleavage and religation and a C-terminal DNA-wrapping domain. The B-subunit (gyrB) contains a ATP hydrolysis at N-terminal domain and C-terminal domain interacts with both Gyrase A and DNA. Another conserved and essential type-II topoisomerase in bacteria, called TopoIV, is primarily responsible for separating the linked closed circular bacterial chromosomes produced in replication. This enzyme relaxes the supercoiled DNA. Topoisomerase IV is a C₂E₂ enzyme, encoded by parC and parE. These subunits parC and parE are highly identical to GyrA and GyrB, respectively. In S. aureus, the identity between GyrB and parE is 52%, where as the identity between GyrA and B is only 5%. The overall sequence identity between gyrase and topoisomerase IV in different bacterial species is high. Therefore, the compounds that target bacterial type-II topoisomerases have the potential to inhibit two targets in cell i.e. DNA gyrase and Topo IV; as is the case in present invention.

The continuous emergence of antibiotic resistance demands the development of novel classes of antibiotics. In pursuit of that goal, the present invention discloses some substituted benzthiazoles and thiazolopyridines compounds useful for the treatment of bacterial infection. WO07/148,093 discloses some benzothiazoles and thiazolopyridines useful as antibacterial agents. WO2007056330 discloses benzimidazole derivatives as Gyrase B inhibitors. WO2007/038367 discloses indazoles, benzothiazoles, benzoisothiazoles, benzisoxazoles, pyrazolopyridines, iosthiazolopyridines and prepatation and uses thereof. WO2006/130613 discloses substituted biarylheterocycle derivatives as protein kinase inhibitors for the treatment of cancer and other diseases. WO2000/075145 discloses cell adhesion-inhibiting anti-inflammatory compounds. WO2006/013095 discloses preparation of arylazolopyridines as p38 kinase inhibitors.

### SUMMARY OF THE INVENTION

The present invention provides substituted benzthiazole and thiazolopyridine compounds having Gyrase B and/or Topo Par E inhibitory activity. The compounds can be used in the treatment or prevention of bacterial infection. Also, provided are processes for synthesizing such compounds.

The compounds of the said invention exhibit activity against strains of Gram-positive, Gram-negative and anaerobic bacteria. Therefore, the compounds of present invention are useful for the treatment of pathologic condition arisen from bacterial infection.

Pharmaceutical compositions containing such compounds are provided together with the pharmaceutically acceptable carriers or diluents, which can be used for the treatment or prevention of bacterial infections. These pharmaceutical compositions may be administered or coadministered by a wide variety of routes including, for example, oral, topical, rectal, internasal, or by parenteral route. The composition may also be administered or coadministered in slow release dosage forms.

Although one specific enantiomer has been shown by way of example, the racemates, diastereomers, N-oxides, polymorphs, pharmaceutically acceptable salts, pharmaceutically acceptable solvates, co-crystals, prodrugs and metabolites having the same type of activity are also provided as well as pharmaceutical compositions comprising the compounds, their metabolites, racemates, enantiomers, N-oxides, polymorphs, solvates, co-crystals, prodrugs or pharmaceutically acceptable salts thereof, in combination with a pharmaceutically acceptable carrier and optionally included excipients.

The therapeutically effective amounts of one or more compounds of the present invention can be used in combination with one or more other therapeutic agents, for example, protein synthesis inhibitors, aminoglycosides, cell wall synthesis inhibitors (glycopeptides, beta-lactams, etc.), RNA and DNA synthesis inhibitors or fatty acid synthesis inhibitors.

Other objects will be set forth in accompanying description and in the part will be apparent from the description or may be learnt by the practice of the invention.

### DETAILED DESCRIPTION OF THE INVENTION

In accordance with one aspect of the invention, are provided compounds having the structure of Formula I

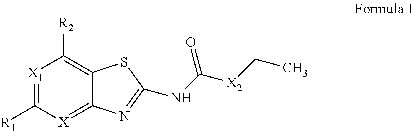

and its pharmaceutically acceptable salts, pharmaceutically acceptable solvates, co-crystals, enantiomers, diastereomers, polymorphs, prodrugs, metabolites or N-oxides wherein

### X is N, —C—F or —CCOOR_{f};

### X₁ is —CH—, N, —C—F or —CCOOR_{f};

### X₂ is —NH— or —O—;

R₁ is cycloalkyl, aryl, heteroaryl or heterocyclyl;
R₂ is hydrogen, alkyl, cycloalkyl, aryl, heteroaryl, heterocyclyl, —NR_{f}R_{q}, —CONR_{f}R_{q}, —COR_{f}, —SO₂R_{f}—COOR_{f}, —CR_{f}═NOR_{f} or —OCONR_{f}R_{q}; wherein R_{f} and R_{q} are independently selected from to hydrogen, alkyl, cycloalkyl, aryl, heteroaryl and heterocyclyl; with the proviso that

when R₂ is —NR_{f}R_{q}, —CH₂NR_{f}R_{q} or —CONR_{f}R_{q} and R_{f} is heterocyclyl or —CH₂-heterocyclyl then said heterocyclyl cannot be 1-aza-bicyclo[2.2.2]octane.

when R₂ is —NR_{f}R_{q} wherein R_{f} is heteroaryl and R₁ is phenyl or pyridinyl then it cannot be further substituted with —CH₂-thiazolidinedione or —CH₂-dialkoxy.

In one embodiment, the present invention provides a compound of Formula Ia,

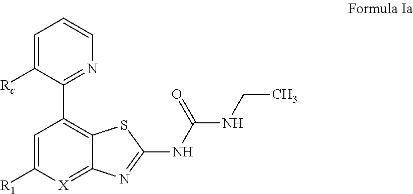

wherein X is —C—F

### R_{c} is H or F

R₁ is cycloalkyl, aryl, heteroaryl or heterocyclyl;

In another embodiment, said alkyl is selected from a branched or unbranched saturated hydrocarbon chain having 1 to 20 carbon atoms, for example, methyl, ethyl, n-propyl, iso-propyl, n-butyl, iso-butyl, t-butyl or n-hexyl.

In another embodiment, said cycloalkyl is selected from cyclopropyl, cyclobutyl, cyclopentyl, cyclohexyl, adamantyl or bicyclo[2.2.1]heptanyl.

In another embodiment, said heteroaryl is selected from monocyclic ring, for example, pyridinyl, pyrimidinyl, thiophenyl, isoxazolyl, oxadiazolyl, furanyl, pyrazolyl, imidazolyl, pyrrolyl, oxazolyl, 1,2,3-triazolyl, thienyl or fused bicyclic ring, for example, benzoimidazolyl, benzofuranyl, indolyl, benzothiazolyl or benzoxazolyl.

In another embodiment, said heterocyclyl is selected from piperidinyl, morpholinyl, pyrrolidinyl, piperazinyl, azabicyclohexyl, oxazolidinyl, tetrahydrofuranyl, tetrahydropyridinyl, dihydropyridinyl, tetrahydrofuranyl or dihydrofuranyl

In another embodiment, said alkyl, aryl, heteroaryl, heterocyclyl or cycloalkyl are either unsubstituted or substituted with one or more substituent, for example, alkyl, cycloalkyl, aryl, heteroaryl, heterocyclyl, hydroxy, alkoxy, oxo, halo, alkylcarbonyl, carboxy, alkoxycarbonyl, aryloxy, heteroaryloxy, heterocyclyloxy, —CO-cycloalkyl, —CO-aryl, —CO-heteroaryl, —CO-heterocyclyl, —NHCO-alkyl, —NR_{f}R_{q}— wherein R_{f} and R_{y} areas defined earlier.

In another embodiment, the invention encompasses compounds that include, for example,
1-Ethyl-3-[4-fluoro-7-(3-fluoropyridin-2-yl)-5-(pyridin-3-yl)-1,3-benzothiazol-2-yl]urea (Compound No. 1),
1-Ethyl-3-[4-fluoro-7-(3-fluoropyridin-2-yl)-5-(6-methyloxypyridin-3-yl)-1,3-benzothiazol-2-yl]urea (Compound No. 2),
1-Ethyl-3-[4-fluoro-7-(3-fluoropyridin-2-yl)-5-(6-methylpyridin-3-yl)-1,3-benzothiazol-2-yl]urea (Compound No. 3),
1-Ethyl-3-[4-fluoro-7-(3-fluoropyridin-2-yl)-5-(pyridin-4-yl)-1,3-benzothiazol-2-yl]urea (Compound No. 4),
1-Ethyl-3-[4-fluoro-7-(3-fluoropyridin-2-yl)-5-(5-fluoropyridin-3-yl)-1,3-benzothiazol-2-yl]urea (Compound No. 5),
1-Ethyl-3-[4-fluoro-7-(3-fluoropyridin-2-yl)-5-(1-methyl-1H-pyrazol-4-yl)-1,3-benzothiazol-2-yl]urea (Compound No. 6),
1-{5-[6-(4-Acetylpiperazin-1-yl)pyridin-3-yl]-4-fluoro-7-(3-fluoropyridin-2-yl)-1,3-benzothiazol-2-yl}-3-ethylurea (Compound No. 7),
1-Ethyl-3-{4-fluoro-7-(3-fluoropyridin-2-yl)-5-[6-(morpholin-4-yl)pyridin-3-yl]-1,3-benzothiazol-2-yl}urea (Compound No. 8),
1-{5-[6-(Dimethylamino)pyridin-3-yl]-4-fluoro-7-(3-fluoropyridin-2-yl)-1,3-benzothiazol-2-yl}-3-ethylurea (Compound No. 9),
1-Ethyl-3-[4-fluoro-7-(3-fluoropyridin-2-yl)-5-(6-fluoropyridin-3-yl)-1,3-benzothiazol-2-yl]urea (Compound No. 10),
1-Ethyl-3-[4-fluoro-7-(3-fluoropyridin-2-yl)-5-(2-methoxypyrimidin-5-yl)-1,3-benzothiazol-2-yl]urea (Compound No. 11),
1-[5-(6-Aminopyridin-3-yl)-4-fluoro-7-(3-fluoropyridin-2-yl)-1,3-benzothiazol-2-yl]-3-ethylurea (Compound No. 12),
1-Ethyl-3-[4-fluoro-7-(3-fluoropyridin-2-yl)-5-(pyrimidin-5-yl)-1,3-benzothiazol-2-yl]urea (Compound No. 13),
1-Ethyl-3-[4-fluoro-5-(2-fluoropyridin-4-yl)-7-(3-fluoropyridin-2-yl)-1,3-benzothiazol-2-yl]urea (Compound No. 14),
1-Ethyl-3-{4-fluoro-7-(3-fluoropyridin-2-yl)-5-[6-(piperazin-1-yl)pyridin-3-yl]-1,3-benzothiazol-2-yl}urea (Compound No. 15),
Methyl 4-{2-[(ethylcarbamoyl)amino]-4-fluoro-7-(3-fluoropyridin-2-yl)-1,3-benzothiazol-5-yl}benzoate (Compound No. 16),
Methyl 3-{2-[(ethylcarbamoyl)amino]-4-fluoro-7-(3-fluoropyridin-2-yl)-1,3-benzothiazol-5-yl}benzoate (Compound No. 17),
Methyl (4-{2-[(ethylcarbamoyl)amino]-4-fluoro-7-(3-fluoropyridin-2-yl)-1,3-benzothiazol-5-yl}phenyl)acetate (Compound No. 18),
1-{5-[2-(Cyclopentylamino)pyrimidin-5-yl]-4-fluoro-7-(3-fluoropyridin-2-yl)-1,3-benzothiazol-2-yl}-3-ethylurea (Compound No. 19),
(4-{2-[(Ethylcarbamoyl)amino]-4-fluoro-7-(3-fluoropyridin-2-yl)-1,3-benzothiazol-5-yl}phenyl)acetic acid (Compound No. 20),
4-{2-[(Ethylcarbamoyl)amino]-4-fluoro-7-(3-fluoropyridin-2-yl)-1,3-benzothiazol-5-yl}benzoic acid (Compound No. 21),
3-{2-[(Ethylcarbamoyl)amino]-4-fluoro-7-(3-fluoropyridin-2-yl)-1,3-benzothiazol-5-yl}benzoic acid (Compound No. 22),
1-Ethyl-3-{4-fluoro-7-(3-fluoropyridin-2-yl)-5-[4-(hydroxymethyl)phenyl]-1,3-benzothiazol-2-yl}urea (Compound No. 23),
1-Ethyl-3-{4-fluoro-7-(3-fluoropyridin-2-yl)-5-[3-(hydroxymethyl)phenyl]-1,3-benzothiazol-2-yl}urea (Compound No. 24),
1-Ethyl-3-[4-fluoro-7-(3-fluoropyridin-2-yl)-5-{2-[(2-methoxyethyl)amino]pyrimidin-5-yl}-1,3-benzothiazol-2-yl]urea (Compound No. 25),
1-Ethyl-3-[4-fluoro-7-(3-fluoropyridin-2-yl)-5-{6-[(2-methylpropyl)amino]pyridin-3-yl}-1,3-benzothiazol-2-yl]urea (Compound No. 26),
1-Ethyl-3-{4-fluoro-7-(3-fluoropyridin-2-yl)-5-[2-(4-hydroxypiperidin-1-yl)pyrimidin-5-yl]-1,3-benzothiazol-2-yl}urea (Compound No. 27),
1-Ethyl-3-{4-fluoro-7-(3-fluoropyridin-2-yl)-5-[2-(piperidin-1-yl)pyrimidin-5-yl]-1,3-benzothiazol-2-yl}urea (Compound No. 28),
1-Ethyl-3-{4-fluoro-7-(3-fluoropyridin-2-yl)-5-[2-(pyrrolidin-1-yl)pyrimidin-5-yl]-1,3-benzothiazol-2-yl}urea (Compound No. 29),
Ethyl 1-(5-{2-[(ethylcarbamoyl)amino]-4-fluoro-7-(3-fluoropyridin-2-yl)-1,3-benzothiazol-5-yl}pyrimidin-2-yl)piperidine-4-carboxylate (Compound No. 30),
Methyl 5-{2-[(ethylcarbamoyl)amino]-4-fluoro-7-(3-fluoropyridin-2-yl)-1,3-benzothiazol-5-yl}pyridine-2-carboxylate (Compound No. 31),
1-Ethyl-3-{4-fluoro-7-(3-fluoropyridin-2-yl)-5-[2-(4-oxopiperidin-1-yl)pyrimidin-5-yl]-1,3-benzothiazol-2-yl}urea (Compound No. 32),
Ethyl 3-[4-(5-{2-[(ethylcarbamoyl)amino]-4-fluoro-7-(3-fluoropyridin-2-yl)-1,3-benzothiazol-5-yl}pyrimidin-2-yl)piperazin-1-yl]propanoate (Compound No. 33),
1-Ethyl-3-[4-fluoro-7-(3-fluoropyridin-2-yl)-5-{2-[4-(hydroxymethyl)piperidin-1-yl]pyrimidin-5-yl}-1,3-benzothiazol-2-yl]urea (Compound No. 34),
1-Ethyl-3-[4-fluoro-7-(3-fluoropyridin-2-yl)-5-{2-[3-(hydroxymethyl)piperidin-1-yl]pyrimidin-5-yl}-1,3-benzothiazol-2-yl]urea (Compound No. 35),
1-Ethyl-3-[4-fluoro-7-(3-fluoropyridin-2-yl)-5-phenyl-1,3-benzothiazol-2-yl]urea (Compound No. 36),
1-Ethyl-3-[4-fluoro-7-(pyridin-2-yl)-5-(pyridin-3-yl)-1,3-benzothiazol-2-yl]urea (Compound No. 37),
1-Ethyl-3-[4-fluoro-5-(6-methylpyridin-3-yl)-7-(pyridin-2-yl)-1,3-benzothiazol-2-yl]urea (Compound No. 38),
1-Ethyl-3-[4-fluoro-5-(6-methoxypyridin-3-yl)-7-(pyridin-2-yl)-1,3-benzothiazol-2-yl]urea (Compound No. 39),
1-Ethyl-3-[4-fluoro-7-(pyridin-2-yl)-5-(pyrimidin-5-yl)-1,3-benzothiazol-2-yl]urea (Compound No. 40),
1-Ethyl-3-[4-fluoro-5-(1-methyl-1H-pyrazol-4-yl)-7-(pyridin-2-yl)-1,3-benzothiazol-2-yl]urea (Compound No. 41),
Methyl 4-{2-[(ethylcarbamoyl)amino]-4-fluoro-7-(pyridin-2-yl)-1,3-benzothiazol-5-yl}benzoate (Compound No. 42),
Methyl 3-{2-[(ethylcarbamoyl)amino]-4-fluoro-7-(pyridin-2-yl)-1,3-benzothiazol-5-yl}benzoate (Compound No. 43),
Ethyl (3-{2-[(ethylcarbamoyl)amino]-4-fluoro-7-(pyridin-2-yl)-1,3-benzothiazol-5-yl}phenoxy)acetate (Compound No. 44),
Ethyl (4-{2-[(ethylcarbamoyl)amino]-4-fluoro-7-(pyridin-2-yl)-1,3-benzothiazol-5-yl}phenoxy)acetate (Compound No. 45),
Methyl 4-{2-[(ethylcarbamoyl)amino]-4-fluoro-7-(pyridin-2-yl)-1,3-benzothiazol-5-yl}-2-methoxybenzoate (Compound No. 46),
Ethyl 1-[(4-{2-[(ethylcarbamoyl)amino]-4-fluoro-7-(pyridin-2-yl)-1,3-benzothiazol-5-yl}phenyl)carbonyl]piperidine-3-carboxylate (Compound No. 47),
Methyl 5-{2-[(ethylcarbamoyl)amino]-4-fluoro-7-(pyridin-2-yl)-1,3-benzothiazol-5-yl}pyridine-3-carboxylate (Compound No. 48),
Methyl (4-{2-[(ethylcarbamoyl)amino]-4-fluoro-7-(pyridin-2-yl)-1,3-benzothiazol-5-yl}-1H-pyrazol-1-yl)acetate (Compound No. 49),
Methyl 4-{2-[(ethylcarbamoyl)amino]-4-fluoro-7-(pyridin-2-yl)-1,3-benzothiazol-5-yl}thiophene-2-carboxylate (Compound No. 50),
1-Ethyl-3-[4-fluoro-5-(6-{[2-(morpholin-4-yl)ethyl]amino}pyridin-3-yl)-7-(pyridin-2-yl)-1,3-benzothiazol-2-yl]urea (Compound No. 51),
1-Ethyl-3-[4-fluoro-5-{3-[(4-methylpiperazin-1-yl)carbonyl]phenyl}-7-(pyridin-2-yl)-1,3-benzothiazol-2-yl]urea (Compound No. 52),
1-Ethyl-3-{4-fluoro-5-[6-(4-methylpiperazin-1-yl)pyridin-3-yl]-7-(pyridin-2-yl)-1,3-benzothiazol-2-yl}urea (Compound No. 53),
1-Ethyl-3-{4-fluoro-5-[2-(4-methylpiperazin-1-yl)pyridin-4-yl]-7-(pyridin-2-yl)-1,3-benzothiazol-2-yl}urea (Compound No. 54),
1-Ethyl-3-[4-fluoro-5-{4-[(4-methylpiperazin-1-yl)carbonyl]phenyl}-7-(pyridin-2-yl)-1,3-benzothiazol-2-yl]urea (Compound No. 55),
1-Ethyl-3-{4-fluoro-5-[2-(piperazin-1-yl)pyridin-3-yl]-7-(pyridin-2-yl)-1,3-benzothiazol-2-yl}urea (Compound No. 56),
1-[5-{6-[3-(Dimethylamino)propoxy]pyridin-3-yl}-4-fluoro-7-(pyridin-2-yl)-1,3-benzothiazol-2-yl]-3-ethylurea (Compound No. 57),
1-Ethyl-3-[4-fluoro-5-{2-[3-(hydroxymethyl)piperidin-1-yl]pyrimidin-5-yl}-7-(pyridin-2-yl)-1,3-benzothiazol-2-yl]urea (Compound No. 58),
1-Ethyl-3-{4-fluoro-5-[6-(morpholin-4-yl)pyridin-3-yl]-7-(pyridin-2-yl)-1,3-benzothiazol-2-yl}urea (Compound No. 59),
tert-Butyl 4-{2-[(ethylcarbamoyl)amino]-4-fluoro-7-(pyridin-2-yl)-1,3-benzothiazol-5-yl}-3,6-dihydropyridine-1(2H)-carboxylate (Compound No. 60),
Ethyl N-(5-{2-[(ethylcarbamoyl)amino]-4-fluoro-7-(pyridin-2-yl)-1,3-benzothiazol-5-yl}pyrimidin-2-yl)-N-methylglycinate (Compound No. 61),
1-{5-[2-(4-Aminopiperidin-1-yl)pyrimidin-5-yl]-4-fluoro-7-(pyridin-2-yl)-1,3-benzothiazol-2-yl}-3-ethylurea hydrochloride salt (Compound No. 62),
1-Ethyl-3-[4-fluoro-5-{2-[(piperidin-4-ylmethyl)amino]pyrimidin-5-yl}-7-(pyridin-2-yl)-1,3-benzothiazol-2-yl]urea hydrochloride salt (Compound No. 63),
Ethyl N-(5-{2-[(ethylcarbamyl)amino]-4-fluoro-7-(3-fluoropyridin-2-yl)-1,3-benzothiazol-5-yl}pyrimidin-2-yl)glycinate (Compound No. 64),
Methyl 4-{2-[(ethylcarbamoyl)amino]-4-fluoro-7-(3-fluoropyridin-2-yl)-1,3-benzothiazol-5-yl}-2-methoxybenzoate (Compound No. 65),
Ethyl 1-[(4-{2-[(ethylcarbamoyl)amino]-4-fluoro-7-(3-fluoropyridin-2-yl)-1,3-benzothiazol-5-yl}-phenyl)carbonyl]piperidine-3-carboxylate (Compound No. 66),
Ethyl (4-{2-[(ethylcarbamoyl)amino]-4-fluoro-7-(3-fluoropyridin-2-yl)-1,3-benzothiazol-5-yl}phenoxy)acetate (Compound No. 67),
Ethyl (3-{2-[(ethylcarbamoyl)amino]-4-fluoro-7-(3-fluoropyridin-2-yl)-1,3-benzothiazol-5-yl}phenoxy)acetate (Compound No. 68),
Methyl 5-{2-[(ethylcarbamoyl)amino]-4-fluoro-7-(3-fluoropyridin-2-yl)-1,3-benzothiazol-5-yl}pyridine-3-carboxylate (Compound No. 69),
Ethyl N-(5-{2-[(ethylcarbamoyl)amino]-4-fluoro-7-(3-fluoropyridin-2-yl)-1,3-benzothiazol-5-yl}pyrimidin-2-yl)-N-methylglycinate (Compound No. 70),
Diethyl[(5-{2-[(ethylcarbamoyl)amino]-4-fluoro-7-(3-fluoropyridin-2-yl)-1,3-benzothiazol-5-yl}pyrimidin-2-yl)amino]propanedioate (Compound No. 71),
Dimethyl N-(5-{2-[(ethylcarbamoyl)amino]-4-fluoro-7-(3-fluoropyridin-2-yl)-1,3-benzothiazol-5-yl}pyrimidin-2-yl)-L-glutamate (Compound No. 72),
1-Ethyl-3-[4-fluoro-7-(3-fluoropyridin-2-yl)-5-{2-[(piperidin-4-ylmethyl)amino]pyrimidin-5-yl}-1,3-benzothiazol-2-yl]urea hydrochloride salt (Compound No. 73),
1-{5-[2-(3-Aminopyrrolidin-1-yl)pyrimidin-5-yl]-4-fluoro-7-(3-fluoropyridin-2-yl)-1,3-benzothiazol-2-yl}-3-ethylurea hydrochloride salt (Compound No. 74),
1-Ethyl-3-{4-fluoro-7-(3-fluoropyridin-2-yl)-5-[2-(piperazin-1-yl)pyrimidin-5-yl]-1,3-benzothiazol-2-yl}urea hydrochloride salt (Compound No. 75),
1-Ethyl-3-{4-fluoro-7-(3-fluoropyridin-2-yl)-5-[2-(piperidin-4-ylamino)pyrimidin-5-yl]-1,3-benzothiazol-2-yl}urea hydrochloride salt (Compound No. 76),
1-{5-[2-(4-Aminopiperidin-1-yl)pyrimidin-5-yl]-4-fluoro-7-(3-fluoropyridin-2-yl)-1,3-benzothiazol-2-yl}-3-ethylurea hydrochloride salt (Compound No. 77),
1-[5-{2-[2-(1,2-Dihydroxy-ethyl)-4,5-dihydroxy-tetrahydro-furan-3-yloxy]-pyrimidin-5 yl}-4-fluoro-7-(3-fluoro-pyridin-2yl)-benzothiazol-2yl]-3-ethyl-urea (Compound No. 78),
1-(5-{2-[5-(1,2-Dihydroxy-ethyl)-2,2-dimethyl-tetrahydro-furo[2,3-d][1,3]dioxol-6-yloxy]-pyrimidine-5-yl}-4-fluoro-7-pyridin-2-yl-benzpthiazol-2-yl)-3-ethyl-urea (Compound No. 79),
1-{5-[2-(2,3-Dihydroxypropoxy)pyrimidin-5-yl]-4-fluoro-7-(3-fluoropyridin-2-yl)-1,3-benzothiazol-2-yl}-3-ethylurea (Compound No. 80),
1-Ethyl-3-[4-fluoro-7-(3-fluoropyridin-2-yl)-5-{1-[2-(morpholin-4-yl)ethyl]-1H-pyrazol-4-yl}-1,3-benzothiazol-2-yl]urea hydrochloride salt (Compound No. 81),
1-Ethyl-3-{4-fluoro-5-[6-(piperazin-1-yl)pyridin-3-yl]-7-(pyridin-2-yl)-1,3-benzothiazol-2-yl}urea (Compound No. 82),
1-Ethyl-3-[4-fluoro-7-(3-fluoropyridin-2-yl)-5-{4-[(4-methylpiperazin-1-yl)carbonyl]phenyl}-1,3-benzothiazol-2-yl]urea (Compound No. 83),
1-Ethyl-3-[4-fluoro-5-(1H-pyrazol-4-yl)-7-(pyridin-2-yl)-1,3-benzothiazol-2-yl]urea Hydrochloride salt (Compound No. 84),
1-Ethyl-3-[4-fluoro-7-(pyridin-2-yl)-5-(1,2,3,6-tetrahydropyridin-4-yl)-1,3-benzothiazol-2-yl]urea Hydrochloride salt (Compound No. 85),
1-Ethyl-3-{4-fluoro-5-[2-(piperazin-1-yl)pyrimidin-5-yl]-7-(pyridin-2-yl)-1,3-benzothiazol-2-yl}urea hydrochloride salt (Compound No. 86),
1-{5-[2-(3-Aminopyrrolidin-1-yl)pyrimidin-5-yl]-4-fluoro-7-(pyridin-2-yl)-1,3-benzothiazol-2-yl}-3-ethylurea hydrochloride salt (Compound No. 87),
1-Ethyl-3-{4-fluoro-5-[2-(piperidin-4-ylamino)pyrimidin-5-yl]-7-(pyridin-2-yl)-1,3-benzothiazol-2-yl}urea hydrochloride salt (Compound No. 88),
Methyl 5-{2-[(ethylcarbamoyl)amino]-4-fluoro-7-(pyridin-2-yl)-1,3-benzothiazol-5-yl}pyridine-2-carboxylate (Compound No. 89),
Methyl (4-{2-[(ethylcarbamoyl)amino]-4-fluoro-7-(pyridin-2-yl)-1,3-benzothiazol-5-yl}phenyl)acetate (Compound No. 90),
Diethyl N-(5-{2-[(ethylcarbamoyl)amino]-4-fluoro-7-(pyridin-2-yl)-1,3-benzothiazol-5-yl}pyrimidin-2-yl)aspartate (Compound No. 91),
Diethyl N-(5-{2-[(ethylcarbamoyl)amino]-4-fluoro-7-(pyridin-2-yl)-1,3-benzothiazol-5-yl}pyrimidin-2-yl)-L-glutamate (Compound No. 92),
4-{2-[(Ethylcarbamoyl)amino]-4-fluoro-7-(pyridin-2-yl)-1,3-benzothiazol-5-yl}benzoic acid Lithium salt (Compound No. 93),
3-{2-[(Ethylcarbamoyl)amino]-4-fluoro-7-(pyridin-2-yl)-1,3-benzothiazol-5-yl}benzoic acid Lithium salt (Compound No. 94),
(3-{2-[(Ethylcarbamoyl)amino]-4-fluoro-7-(pyridin-2-yl)-1,3-benzo thiazol-5-yl}phenoxy)acetic acid Lithium salt (Compound No. 95),
(4-{2-[(Ethylcarbamoyl)amino]-4-fluoro-7-(pyridin-2-yl)-1,3-benzothiazol-5-yl}phenoxy)acetic acid Lithium salt (Compound No. 96),
4-{2-[(Ethylcarbamoyl)amino]-4-fluoro-7-(pyridin-2-yl)-1,3-benzothiazol-5-yl}-2-methoxybenzoic acid (Compound No. 97),
1-[(4-{2-[(Ethylcarbamoyl)amino]-4-fluoro-7-(pyridin-2-yl)-1,3-benzothiazol-5-yl}phenyl)carbonyl]piperidine-3-carboxylic acid Lithium salt (Compound No. 98),
5-{2-[(Ethylcarbamoyl)amino]-4-fluoro-7-(pyridin-2-yl)-1,3-benzothiazol-5-yl}pyridine-3-carboxylic acid Lithium salt (Compound No. 99),
(4-{2-[(Ethylcarbamoyl)amino]-4-fluoro-7-(pyridin-2-yl)-1,3-benzothiazol-5-yl}-1H-pyrazol-1-yl)acetic acid lithium salt (Compound No. 100),
4-{2-[(Ethylcarbamoyl)amino]-4-fluoro-7-(pyridin-2-yl)-1,3-benzothiazol-5-yl}thiophene-2-carboxylic acid Lithium salt (Compound No. 101),
5-{2-[(Ethylcarbamoyl)amino]-4-fluoro-7-(pyridin-2-yl)-1,3-benzothiazol-5-yl}pyridine-2-carboxylic acid Lithium salt (Compound No. 102),
(4-{2-[(Ethylcarbamoyl)amino]-4-fluoro-7-(pyridin-2-yl)-1,3-benzothiazol-5-yl}phenyl)acetic acid Lithium salt (Compound No. 103),
1-(5-{2-[(Ethylcarbamoyl)amino]-4-fluoro-7-(pyridin-2-yl)-1,3-benzothiazol-5-yl}pyrimidin-2-yl)piperidine-4-carboxylic acid Lithium salt (Compound No. 104),
5-{2-[(1-Carboxy-2-methoxy-2-oxoethyl)amino]pyrimidin-5-yl}-2-[(ethylcarbamoyl)amino]-4-fluoro-7-(pyridin-2-yl)-1,3-benzothiazole Lithium salt (Compound No. 105),
N-(5-{2-[(Ethylcarbamoyl)amino]-4-fluoro-7-(pyridin-2-yl)-1,3-benzothiazol-5-yl}pyrimidin-2-yl)aspartic acid Lithium salt (Compound No. 106),
N-(5-{2-[(Ethylcarbamoyl)amino]-4-fluoro-7-(pyridin-2-yl)-1,3-benzothiazol-5-yl}pyrimidin-2-yl)glycine Lithium salt (Compound No. 107),
4-[(3-{2-[(Ethylcarbamoyl)amino]-4-fluoro-7-(pyridin-2-yl)-1,3-benzothiazol-5-yl}phenyl)amino]-4-oxobutanoic acid Lithium salt (Compound No. 108),
4-{2-[(Ethylcarbamoyl)amino]-4-fluoro-7-(3-fluoropyridin-2-yl)-1,3-benzothiazol-5-yl}-2-methoxybenzoic acid (Compound No. 109),
1-[(4-{2-[(Ethylcarbamoyl)amino]-4-fluoro-7-(3-fluoropyridin-2-yl)-1,3-benzothiazol-5-yl}phenyl)carbonyl]piperidine-3-carboxylic acid Lithium salt (Compound No. 110),
(4-{2-[(Ethylcarbamoyl)amino]-4-fluoro-7-(3-fluoropyridin-2-yl)-1,3-benzothiazol-5-yl}phenoxy)acetic acid Lithium salt (Compound No. 111),
5-{2-[(Ethylcarbamoyl)amino]-4-fluoro-7-(3-fluoropyridin-2-yl)-1,3-benzothiazol-5-yl}pyridine-3-carboxylic acid Lithium salt (Compound No. 112),
N-(5-{2-[(Ethylcarbamoyl)amino]-4-fluoro-7-(3-fluoropyridin-2-yl)-1,3-benzothiazol-5-yl}pyrimidin-2-yl)glycine Lithium salt (Compound No. 113),
N-(5-{2-[(Ethylcarbamoyl)amino]-4-fluoro-7-(3-fluoropyridin-2-yl)-1,3-benzothiazol-5-yl}pyrimidin-2-yl)-N-methylglycine Lithium salt (Compound No. 114),
(3-{2-[(Ethylcarbamoyl)amino]-4-fluoro-7-(3-fluoropyridin-2-yl)-1,3-benzothiazol-5-yl}phenoxy)acetic acid Lithium salt (Compound No. 115),
N-(5-{2-[(Ethylcarbamoyl)amino]-4-fluoro-7-(pyridin-2-yl)-1,3-benzothiazol-5-yl}pyrimidin-2-yl)-N-methylglycine Lithium salt (Compound No. 116),
Methyl[4-(5-{2-[(ethylcarbamoyl)amino]-4-fluoro-7-(pyridin-2-yl)-1,3-benzothiazol-5-yl}pyridin-2-yl)piperazin-1-yl]acetate hydrochloride salt (Compound No. 117),
[4-(5-{2-[(Ethylcarbamoyl)amino]-4-fluoro-7-(pyridin-2-yl)-1,3-benzothiazol-5-yl}pyrimidin-2-yl)piperazin-1-yl]acetic acid Lithium salt (Compound No. 118),
1-Ethyl-3-[4-fluoro-5-{2-[4-(2-hydroxyethyl)piperazin-1-yl]pyrimidin-5-yl}-7-(pyridin-2-yl)-1,3-benzothiazol-2-yl]urea hydrochloride salt (Compound No. 119),
3-[4-(5-{2-[(Ethylcarbamoyl)amino]-4-fluoro-7-(pyridin-2-yl)-1,3-benzothiazol-5-yl}pyrimidin-2-yl)piperazin-1-yl]propanoic acid (Compound No. 120),
3-[4-(5-{2-[(Ethylcarbamoyl)amino]-4-fluoro-7-(pyridin-2-yl)-1,3-benzothiazol-5-yl}pyrimidin-2-yl)piperazin-1-yl]propanoic acid Lithium salt (Compound No. 121),
Ethyl 3-[4-(5-{2-[(ethylcarbamoyl)amino]-4-fluoro-7-(pyridin-2-yl)-1,3-benzothiazol-5-yl}pyridin-2-yl)piperazin-1-yl]propanoate hydrochloride salt (Compound No. 122),
1-Ethyl-3-[4-fluoro-5-{4-[(4-methylpiperazin-1-yl)methyl]phenyl}-7-(pyridin-2-yl)-1,3-benzothiazol-2-yl]urea (Compound No. 123),
1-Ethyl-3-{4-fluoro-5-[4-(morpholin-4-ylmethyl)phenyl]-7-(pyridin-2-yl)-1,3-benzothiazol-2-yl}urea hydrochloride salt (Compound No. 124),
1-Ethyl-3-[4-fluoro-5-(4-{[4-(2-hydroxyethyl)piperazin-1-yl]methyl}phenyl)-7-(pyridin-2-yl)-1,3-benzothiazol-2-yl]urea hydrochloride salt (Compound No. 125),
1-Ethyl-3-[4-fluoro-5-{4-[(4-hydroxypiperidin-1-yl)methyl]phenyl}-7-(pyridin-2-yl)-1,3-benzothiazol-2-yl]urea hydrochloride salt (Compound No. 126),
1-Ethyl-3-[4-fluoro-5-(4-{[4-(hydroxymethyl)piperidin-1-yl]methyl}phenyl)-7-(pyridin-2-yl)-1,3-benzothiazol-2-yl]urea hydrochloride salt (Compound No. 127),
1-Ethyl-3-[4-fluoro-5-(5-{[4-(2-hydroxyethyl)piperazin-1-yl]methyl}thiophen-3-yl)-7-(pyridin-2-yl)-1,3-benzothiazol-2-yl]urea hydrochloride salt (Compound No. 128),
Ethyl 1-[(4-{2-[(ethylcarbamoyl)amino]-4-fluoro-7-(pyridin-2-yl)-1,3-benzothiazol-5-yl}thiophen-2-yl)methyl]piperidine-4-carboxylate hydrochloride salt (Compound No. 129),
Methyl N-[(4-{2-[(ethylcarbamoyl)amino]-4-fluoro-7-(pyridin-2-yl)-1,3-benzothiazol-5-yl}thiophen-2-yl)methyl]-L-serinate hydrochloride salt (Compound No. 130),
Ethyl N-[(4-{2-[(ethylcarbamoyl)amino]-4-fluoro-7-(pyridin-2-yl)-1,3-benzothiazol-5-yl}thiophen-2-yl)methyl]-L-alaninate hydrochloride salt (Compound No. 131),
1-Ethyl-3-[4-fluoro-5-{5-[(4-hydroxypiperidin-1-yl)methyl]thiophen-3-yl}-7-(pyridin-2-yl)-1,3-benzothiazol-2-yl]urea hydrochloride salt (Compound No. 132),
1-Ethyl-3-{4-fluoro-5-[5-({[2-(morpholin-4-yl)ethyl]amino}methyl)thiophen-3-yl]-7-(pyridin-2-yl)-1,3-benzothiazol-2-yl}urea hydrochloride salt (Compound No. 133),
1-[5-(5-{[bis(2-Hydroxyethyl)amino]methyl}thiophen-3-yl)-4-fluoro-7-(pyridin-2-yl)-1,3-benzothiazol-2-yl]-3-ethylurea hydrochloride salt (Compound No. 134),
1-Ethyl-3-{4-fluoro-5-[5-(morpholin-4-ylmethyl)thiophen-3-yl]-7-(pyridin-2-yl)-1,3-benzothiazol-2-yl}urea hydrochloride salt (Compound No. 135),
1-Ethyl-3-[4-fluoro-5-{5-[(4-methylpiperazin-1-yl)methyl]thiophen-3-yl}-7-(pyridin-2-yl)-1,3-benzothiazol-2-yl]urea hydrochloride salt (Compound No. 136),
1-Ethyl-3-[4-fluoro-5-(5-{[4-(2-hydroxyethyl)piperazin-1-yl]methyl}thiophen-2-yl)-7-(pyridin-2-yl)-1,3-benzothiazol-2-yl]urea hydrochloride salt (Compound No. 137) or
Ethyl 1-[(5-{2-[(ethylcarbamoyl)amino]-4-fluoro-7-(pyridin-2-yl)-1,3-benzothiazol-5-yl}thiophen-2-yl)methyl]piperidine-4-carboxylate hydrochloride salt (Compound No. 138)

In yet another aspect, provided herein are pharmaceutical compositions comprising therapeutically effective amounts of one or more compounds described herein together with one or more pharmaceutically acceptable carriers, excipients, or diluents.

In another aspect, provided herein are methods for treating or preventing conditions caused by or contributed to by bacterial infections comprising administering to a mammal in need thereof therapeutically effective amount of one or more compounds of Formula I described herein.

The methods may include one or more of the following embodiments. For example, the condition can be selected from community acquired pneumonia, upper or lower respiratory tract infections, complicated skin and skin structure infections (cSSSI), uncomplicated skin and soft structure infections, hospital acquired (nosocomial) infections, urinary tract infections, intra-abdominal infections, enterococci infections, bacteraemia infections with known or suspected endocarditis, nosocomial bone or joint infections, acne vulgaris, mastitis, catheter infection, foreign body, prosthesis infections or peptic ulcer disease.

In another embodiment, the bacterial infections can be caused by gram positive, gram negative or anaerobic bacteria.

In yet another embodiment, the gram positive, gram negative or anaerobic bacteria can be selected from Staphylococci, Streptococci, Enterococci, Haemophilus, Pseudomonas spp., Klebsiella spp., Moraxalla spp., Chlamydia spp., Mycoplasm spp., Legionella spp., Mycobacterium spp., Helicobacter, Clostridium spp., Bacteroides spp., Corynebacterium, Bacillus spp., Enterobactericeae (E. coli, Klebsiella spp., Proteus spp., etc)

In another embodiment, the bacterium is cocci.

In another embodiment, the cocci are drug resistant.

In another embodiment, the drug resistant cocci are selected from methicillin resistant Staphylococcus aureus (MRSA), vancomycin resistant S. aureus (VRSA), methicillin resistant Staphylococcus epidermidis (MRSE), Streptococcus pyogenes (erm, mef, telithromycin resistance), Enterococcus faecalis and faecium (vancomycin and telithromycin resistance), penicillin resistant Streptococcus pneumoniae (PRSP), and multi-drug resistant Streptococcus pneumoniae.

Also, provided herein are methods for treating, preventing or inhibiting nosocomial and/or community acquired bacterial infection or a associated disease, disorder or infection thereof, comprising administering to a mammal in need thereof, a therapeutically effective amount of one or more fluorobenzthiazole compounds of pharmaceutically acceptable salts, esters, polymorphs, pharmaceutically acceptable solvates, co-crystals, enantiomers, diastereomers, N-oxides, prodrugs or metabolites thereof, in combination with one or more therapeutic agents selected from other antibacterial compounds, for example, protein synthesis inhibitors (linezolid, telithromycin, tigecycline, etc,) aminoglycosides (gentamycin, kanamycin, etc), cell wall synthesis inhibitors (glycopeptides such as vancomycin, teicoplanin, telavancin, bleomycin, etc, beta-lactams, such as penicillin, cephalosporins, carbapenems, etc.), RNA and DNA synthesis inhibitors (quinolones such as nalidixic acid, oxolinic acid etc, fluoquinolones such as ciprofloxacin, levofloxacin, ofloxacin, moxifloxacin, etc.) fatty acid synthesis inhibitors and its derivatives and other therapeutic agents, which can be used to treat, prevent or inhibit nosocomial and community acquired bacterial infection or a associated disease, disorder or infection thereof.

Also, provided herein are methods for treating or preventing acne vulgaris and inflammatory conditions thereof comprising administering to a mammal in need thereof therapeutically effective amounts of one or more compounds of Formula I in combination with one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid, and other therapeutic agents, which can be used to treat acne or condition the skin.

Also, provided herein is the use of a pharmaceutical composition of the combination of the compounds of the said invention with various other therapeutic agents as described above in the manufacture of a medicament for treating, preventing or inhibiting nosocomial or community acquired bacterial infection or any associated disease, disorder or infection thereof.

### DEFINITIONS

The term “protecting group” is used herein to refer to known moieties which have the desirable property of preventing specific chemical reaction at a site on the molecule undergoing chemical modification intended to be left unaffected by the particular chemical modification. Also the term “protecting group”, unless or otherwise specified, may be used with groups such as hydroxy, amino, and carboxy. The examples of such groups are found in T. W. Greene and P. G. M. Wuts, “Protective groups in organic synthesis”, 3^{rd} ed., John Wiley and Sons Inc., New York, 1999, which is incorporated herein by reference.

The term “pharmaceutically acceptable salts” refers to the inorganic and organic base or acid addition salts of compounds of present invention. These salts can be prepared in situ during the final isolation and purification of the compounds or by separately reacting the purified compound in its free form with a suitable organic or inorganic base or acid and isolating the salt thus obtained. Representative salts include, but not limited to, trifluoroacetate, hydrochloride, acetate, fumarate, phosphate, tosylate, hydrobromide, sulfate, bisulfate, nitrate, oxalate, valerate, oleate, palmitate, stearate, laurate, borate, benzoate, lactate, citrate, maleate, succinate, tartrate, naphthylate, mesylate, glucoheptonate, lactobionate, laurylsulfonate and the like. Where the compounds carry acidic moiety, the salts derived from inorganic bases include, but not limited to, lithium, sodium, potassium, calcium, magnesium, zinc, aluminium as well as non-toxic ammonium, quaternary ammonium and amine cations, including, but not limited to ammonium, tetramethylammonium, tetraethylammonium, methylamine, triethylamine, ethylamine, diethylamine, and the like. The salts derived from organic bases include, but not limited to, salts of natural or synthetic amino acids, betaine, caffeine, 2-diethylaminoethanol, N-ethylmorpholine, glucosamine, dibenzylethylene-diamine, chloroprocaine, choline, diethanolamine, ethylenediamine, piperazine, procaine, purine, tromethamine and the like. The free base form may be regenerated by contacting the salt form with a base. While the free base form may differ from the salt form in terms of physical properties, such as solubility, the salts are equivalent to their respective free bases for the purposes of the present invention.

The term “pharmaceutically acceptable solvates” refers to solvates with water (i.e., hydrates) or pharmaceutically acceptable solvents, for example solvates with ethanol and the like. Such solvates are also encompassed within the scope of the disclosure. Furthermore, some of the crystalline forms for compounds described herein may exist as polymorphs and as such are intended to be included in the scope of the disclosure.

The present invention within its scope also includes ‘prodrugs’ of these agents. In general, such prodrugs will be functional derivatives of these compounds, which are readily convertible in vivo into the active drugs. Conventional procedure for the selection and preparation of suitable prodrug derivatives are described, for example, in “Targeted prodrug design to optimize drug delivery”, AAPS PharmSci. 2000, 2(1), E6.

The term “pharmaceutically acceptable carriers” is intended to include non-toxic, inert solid, semi-solid or liquid filler, diluent, encapsulating material or formulation auxiliary of any type.

The term “co-crystals” defines the crystalline phase wherein at least two components of the crystal interact by hydrogen bonding and possibly by other non-covalent interactions rather than by ion pairing.

The term “polymorphs” refers to all crystalline forms and amorphous forms of the compounds described herein. In addition, some of the compounds described herein may form solvates with water or common organic solvents. Such solvates are also encompassed within the scope of this invention.

The term “Community-acquired infections” relates to the infections acquired from the community in the patients, who had not recently been in a health care facility or been in contact with someone who had been recently in a health care facility. Community-acquired respiratory tract infection (CARTI) is a common cause of acute illness in adults and includes, community acquired pneumonia, mild to severe upper and lower respiratory tract infections, acute bronchitis, chronic obstructive pulmonary disease.

The term “Hospital-acquired infections (nosocomial infections)” also known as health-care associated infections relates to the infections acquired by patients from the surrounding bacterial pool in hospital setup. Patients contract these infections from pathogens on the hands of medical personnel, invasive procedures (e.g., intubations and extended ventilation, indwelling vascular lines, urine catheterization), or contaminated air-conditioning systems, contaminated water systems. Most serious hospital acquired infections include ventilator-associated pneumonia (VAP), lower respiratory infection, catheter related infection, foreign body, prosthesis infections or peptic ulcer disease, skin, soft tissue, and surgical-site infections.

In another aspect, the compounds disclosed herein may be prepared by the following reaction sequences as depicted in scheme I-IV

The compound of Formula XVII can be prepared by following scheme I

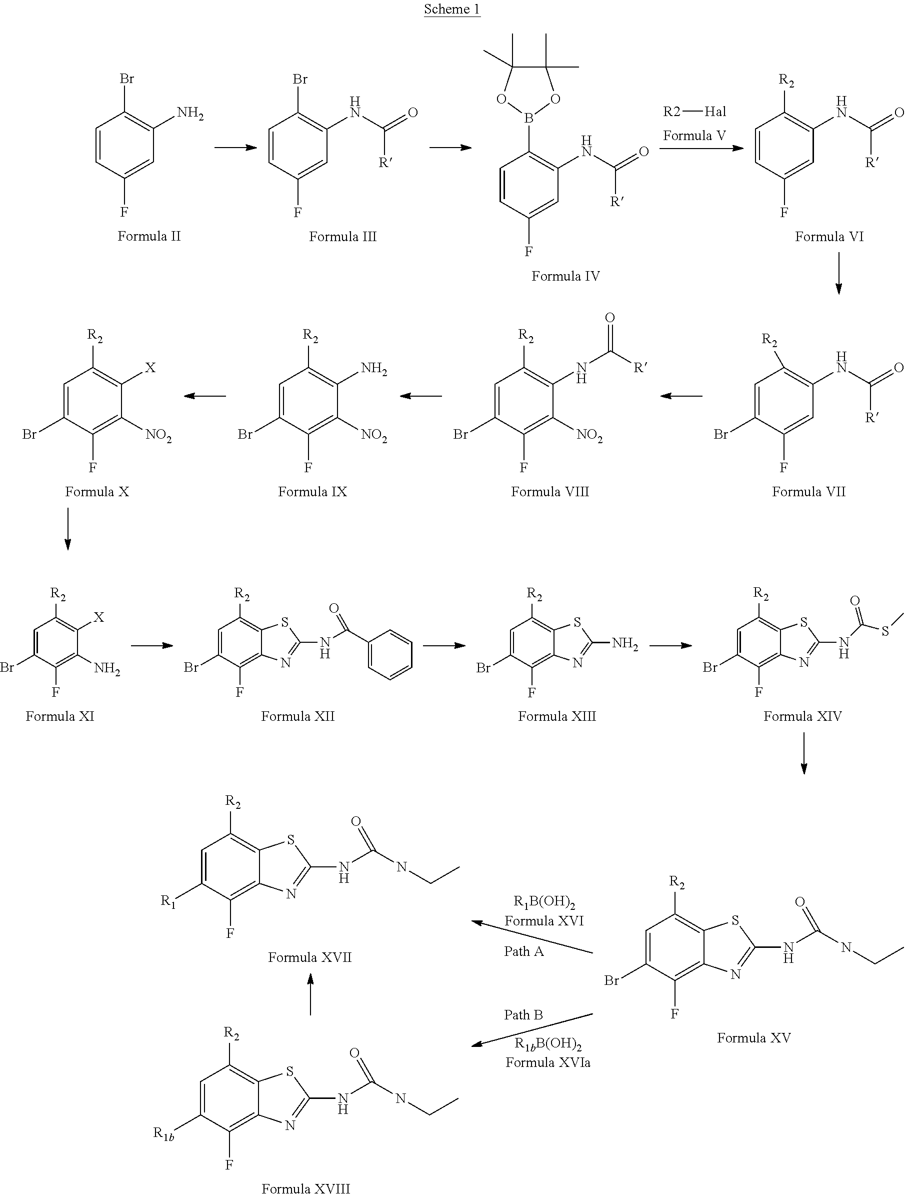

The compound of Formula XVII (wherein R₁ and R₂ are as defined earlier) can be prepared by following the synthetic route as depicted in Scheme I. Thus, a compound of Formula II can be acylated to give a compound of Formula III (wherein R′ is alkyl, haloalkyl or aryl), which can be borylated to a compound of Formula IV. The compound of Formula IV can be coupled with the compound of Formula V to form the compound of Formula VI. The compound of Formula VI can be brominated to give a compound of Formula VII, which can be nitrated to form compound of Formula VIII. Compound of Formula VIII can be N-deacylated to form compounds of Formula IX and then can be halogenated to give a compound of Formula X (wherein X can be halogen). Compound of Formula X can be reduced to give a compound of Formula XI which can be reacted with benzoylisothiocyanate to give a compound of Formula XII. Compound of Formula XII is hydrolyzed to give a compound of Formula XIII that can be reacted with methyl (chloro)thioformate or phenylchloroformate to give a compound of Formula XIV. Compound of Formula XIV is converted to a compound of Formula XV. Compound of Formula XV can directly be coupled with a compound of Formula XVI to give a compound of Formula XVII (Path A). Alternatively, compound of Formula XV can first couple with a compound of Formula XVIa (wherein R_{1b} is a cycloalkyl, aryl, heteroaryl or heterocyclyl protected with a suitable protecting group, for example, t-butyl carbamate (t-Boc), trityl chloride, 9-fluorenyl-methyl carbamate (F-moc), allyloxycarbonyl, trifluoroacetamide or tosyl) to give a compound of Formula XVIII, which is then deprotected to give a compound of Formula XVII (Path B).

N-acylation of a compound of Formula II to form a compound of Formula III can be carried out with acylating agents, for example, pivaloyl chloride, trifluoroacetic anhydride, benzoyl chloride or acetyl chloride in presence of a base, for example, triethylamine or pyridine in an organic solvent, for example, dichloromethane, methylene chloride, chloroform, diethyl ether, tetrahydrofuran or mixture(s) thereof.

Borylation of a compound of Formula III to form a compound of Formula IV can be carried out with bispinacolato diboron or another suitable boron precursor, for example, 9-borabicyclo[3.3.1]nonane (9BBN) in the presence of an appropriate Pd(II) catalyst, for example, [bis-(diphenyl-phosphino)ferrocene palladium II dichloride (Pd(dppf)Cl₂, tetrakistriphenylphosphine palladium (0) [Pd (Ph₃P)₄], Palladium acetate or dichlorobistriphenylphosphine palladium (II), with a suitable base, for example, potassium acetate, sodium acetate or potassium carbonate in one or more solvent, for example, dioxane, toluene, tetrahydrofuran, acetone or dimethylformamide.

Alternatively, borylation of a compound of Formula III to form a compound of Formula IV can also be carried out through synthesis of boronic acid derivative by treating with trimethyl borate, triisopropyl borate or with other borate ester in the presence of base, for example, butyllithium in a solvent, for example, tetrahydrofuran or toluene.

The coupling reaction of the compound of Formula IV with the compound of Formula V to form the compound of Formula VI can be carried out with a base, for example, sodium carbonate, potassium carbonate or cesium carbonate in the presence of a catalyst, for example, dichlorobistriphenylphosphine palladium (II) or tetrakistriphenylphosphine palladium (0) or -(diphenylphosphino)ferrocene palladium II dichloride in a suitable solvent, for example, acetonitrile, water, acetone, 1,4-dioxane, toluene, tetrahydrofuran, dimethylformamide or mixture(s) thereof.

The reaction of the compound of Formula VI to form the compound of Formula VII can be carried out in the presence of a brominating agent, for example, bromine, hypobromous acid, bromine in carbon tetrachloride, pyridinium bromide perbromide, dioxane dibromide or N-bromosuccinimide in the presence of an acid, for example, acetic acid.

Nitration of the compound of Formula VII to give the compound of Formula VIII can be carried out in nitric acid in presence of sulfuric acid or sodium nitrite in the presence of solvent, for example, trifluoroacetic acid, trifluoroacetic anhydride or mixture(s) thereof.

Alternatively, nitration of the compound of Formula VII to give the compound of Formula VIII can be carried out by using nitronium salts, for example, NO₂⁺BF₄⁻, NO₂⁺PF₄⁻, NO₂⁺CF₃SO₃⁻ in the presence of solvent, for example, dichloromethane, acetonitrile, nitromethane or mixture(s) thereof.

N-deacylation of the compound of Formula VIII to give the compound of Formula IX can be carried out in the presence of N-deacylating agents, for example, sulphuric acid, hydrochloric acid, or trifluoroacetic acid.

Halogenation of compound of Formula IX to form the compound of Formula X can be carried out with one or more halogenating agent, for example, iodine or cupric bromide in the presence of isoamylnitrite, sodium nitrite, ethylnitrite, lithium nitrite, potassium nitrite, zinc nitrite, or mixture(s) thereof in a solvent, for example, chloroform, acetonitrile, carbon tetrachloride, methylene chloride or mixture(s) thereof.

Reduction of Compound of Formula X to give a compound of Formula XI can be carried out with reducing agents, for example, metals Fe, Sn or Zn in the presence of an acid, for example, hydrochloric acid in one or more solvents, for example, ethanol, water, methanol, isopropanol or n-propanol.

Alternatively, catalytic reduction of compound of Formula X to give a compound of Formula XI can be done by palladium catalyzed reduction in the presence of H₂ in a solvent, for example, methanol, ethyl acetate, ethanol, water or mixture(s) thereof.

Reaction of compound of Formula XI with benzoylisothiocyanate to give a compound of Formula XII can be carried out in the presence of solvent, for example, acetone, toluene, N-methylpyrrolidone, dimethylformamide, tetrahydrofuran or mixture(s) thereof.

Hydrolysis of compound of Formula XII to give a compound of Formula XIII can be carried out using a base, for example, sodium hydroxide, potassium hydroxide, sodium methoxide or sodium ethoxide in the presence of a solvent, for example, methanol, ethanol, propanol or mixture(s) thereof.

Reaction of a compound of Formula XIII to give a compound of Formula XIV can be carried out with a reagent, for example, methyl(chloro)thioformate, phenylchloroformate or p-nitrophenyl chloroformate in the presence of a base, for example, pyridine or triethylamine optionally in the presence of a solvent, for example, dichloromethane, chloroform, carbon tetrachloride or mixture(s) thereof.

Conversion of compound of Formula XIV to give a compound of Formula XV can be carried out in the presence of a base, for example, ethyl amine, methyl amine or triethylamine in a solvent for example, ethanol, methanol, propanol or mixture(s) thereof.

Path A: Coupling of a compound of Formula XV with a compound of Formula XVI to give a compound of Formula XVII can be carried out using a base, for example, sodium carbonate, potassium carbonate or cesium carbonate in the presence of a catalyst, for example, dichlorobistriphenylphosphine palladium (II) or tetrakistriphenylphosphine palladium (0), -(diphenylphosphino)ferrocene palladium II dichloride in a suitable solvent, for example, acetonitrile, water, acetone, 1,4-dioxane, toluene, dimethylformamide, tetrahydrofuran or mixture(s) thereof.
Path B: Coupling of compound of Formula XV to give a compound of Formula XVIII can be carried out in the same way as coupling of compound of Formula XV to a compound of Formula XVII.

Deprotection of compound of Formula XVIII to give a compound of Formula XVII can be carried out in the presence of one or more acid, for example, hydrochloric acid, trifluoroacetic acid or p-toluene sulfonic acid in one or more organic solvent, for example, ethanol, dichloromethane, acetonitrile, tetrahydrofuran, dioxane or dimethylformamide.

The compounds of Formula XX can be prepared by following scheme II

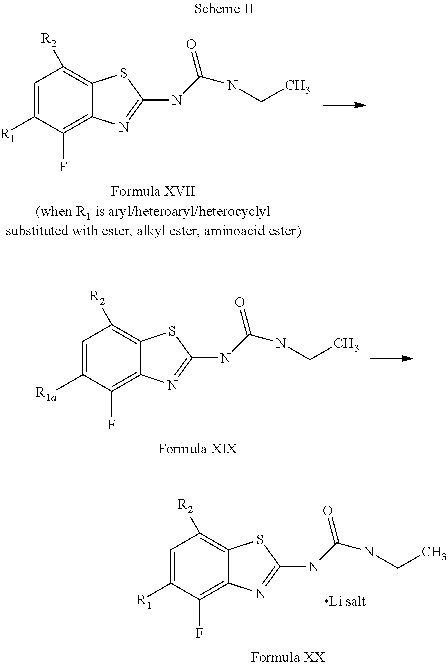

Accordingly, the compounds of Formula XVII (when R₁ is aryl, heteroaryl or heterocyclyl substituted with esters, alkylesters, aminoacid ester) undergo hydrolysis to give a compound of Formula XIX (wherein R₁ₐ is aryl, heteroaryl or heterocyclyl substituted with acid, alkylacid or amino acid), which reacts with lithium hydroxide to give a compound of Formula XX.

Hydrolysis of compounds of Formula XVII to give a compound of Formula XIX can be carried out in the presence of a base, for example, sodium hydroxide, lithium hydroxide or potassium hydroxide in one or more solvent, for example, methanol, ethanol, water, tetrahydrofuran or, acetonitrile.

Reaction of compound of Formula XIX with lithium hydroxide to give a salt of compound of Formula XX can be carried out in the presence of one or more solvents, for example, tetrahydrofuran, water, ethanol, dioxane, acetonitrile, acetone or dimethylformamide.

The compounds of Formulae XXIII and XXV can be prepared by following Scheme III

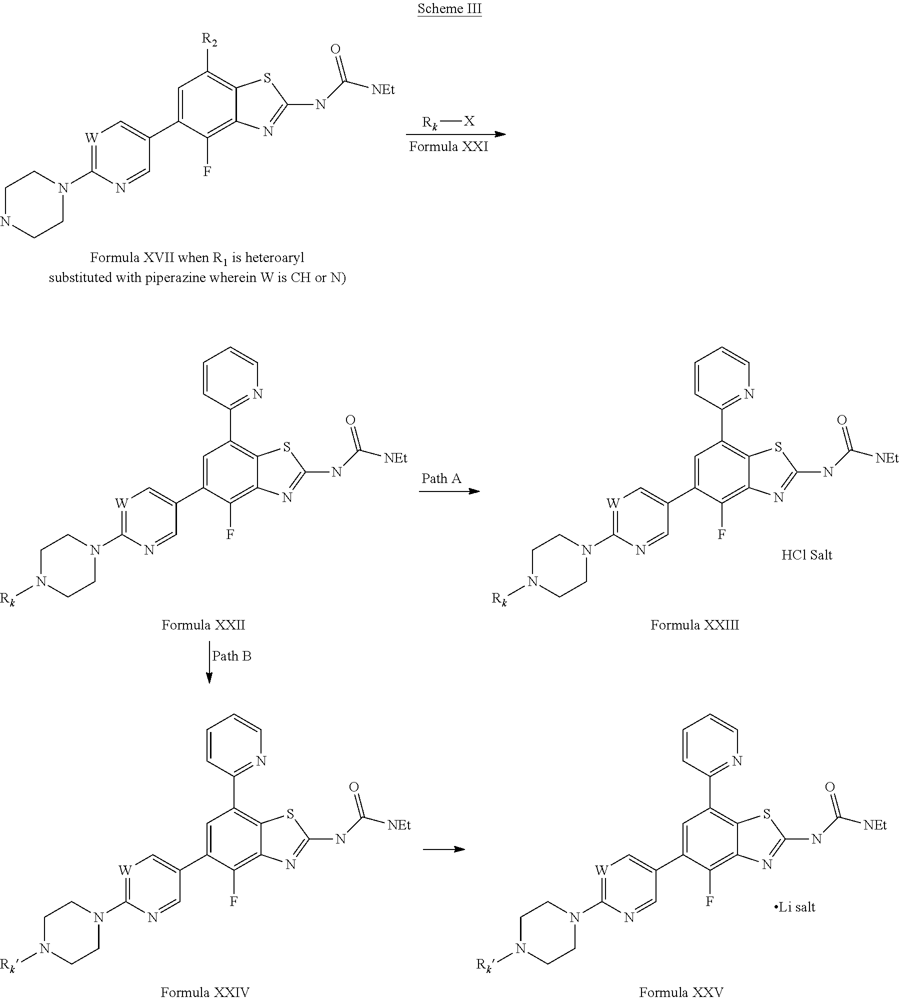

Accordingly, the compound of Formula XIV (when R₁ is heteroaryl substituted with piperazine wherein W is CH or N) couple with a compound of Formula XXI (wherein Rₖ is alkyl substituted with alkoxycarbonyl, hydroxy, amine or substituted amine and X is as defined earlier) to give a compound of Formula XXII, which either undergo salt formation to give a compound of Formula XXIII (Path A) or it first undergoes hydrolysis (when Rₖ is alkyl substituted with alkoxycarbonyl) to give a compound of Formula XXIV (wherein Rₖ′ is alkyl substituted with COOH) and then undergo salt formation to give a compound of Formula XXV.

Coupling of compound of Formula XVII with a compound of Formula XXI to give a compound of Formula XXII can be carried out in the presence of a base, for example, potassium carbonate, sodiumcarbonate, N,N-diisopropylethylamine in one or more solvent, for example, tetrahydrofuran, acetonitrile, acetone, 1,4-dioxane, toluene, dimethylformamide.

Path A: The reaction of compound of Formula XXII with ethanolic hydrochloric acid to give a compound of Formula XXIII can be carried out in one or more solvent for example ethanol, tetrahydrofuran or acetonitrile
Path B: Hydrolysis of compound of Formula XXII to give a compound of Formula XXIV can be carried out in the presence of a base, for example, sodium hydroxide, lithium hydroxide or potassium hydroxide in one or more solvent, for example, methanol, ethanol, water, tetrahydrofuran, or acetonitrile
The reaction of compound of Formula XXIV to give a compound of Formula XXV can be carried out in the similar way as the reaction of compound of Formula XIX to give a compound of Formula XX.

The compound of Formula XXVII can be prepared by following Scheme IV as mentioned below.

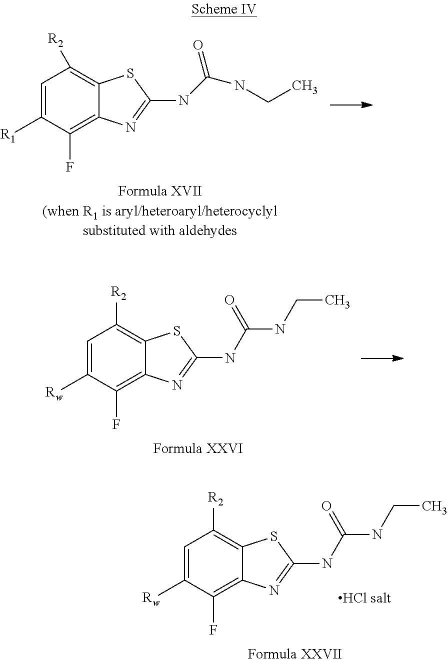

Accordingly, the compound of Formula XVII (when R₁ is aryl, heteroaryl or heterocyclyl substituted with aldehydes) can undergo reductive amination with a primary or secondary amine to give a compound of Formula XXVI (wherein R_{W} is aryl, heteroaryl or heterocyclyl substituted with CH₂-amine) which undergo salt formation to give a compound of Formula XXVII

Reductive amination of compound of Formula XVII with a primary or secondary amine to give a compound of Formula XXVI can be carried out in the presence of acids such as acetic acid or hydrochloric acid and using reducing agent such as sodium cyano borohydride, sodium triacetoxy borohydride or sodium borohydride in one or more solvent, for example, methanol, dichloroethane, ethanol, tetrahydrofuran, or acetonitrile.

Salt formation of compound of Formula XXVI to give a compound of Formula XXVII can be carried out in the similar way as the salt formation of compound of Formula XXII to give a compound of Formula XXIII

In the above schemes, where specific reagents, for example, bases, acids, solvents, condensing agents, acylating agents, hydrolyzing agents, metal catalysts etc., are mentioned, are it is to be understood that other reagents, e.g., other acids, bases, solvents, condensing agents, reducing agent, deprotecting agent, hydrolyzing agents, metal catalysts etc., known to one of ordinary skill in the art may be used. Similarly, reaction temperatures and durations may be adjusted according to the desired needs without undue experimentation and well within the abilities of one of ordinary skill in the art. All the epimers, unless otherwise specified in the above schemes, are also encompassed within the scope of the invention.

Table-1 lists the type of compounds synthesized by using the synthetic procedure as demonstrated in Schemes I-IV.

TABLE I
Formula I

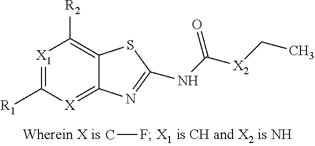

| S. | | |
|---|---|---|
| No. | R₂ | R₁ |
| 1 | 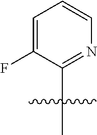 | 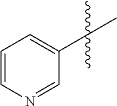 |
| 2 | 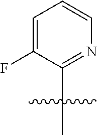 | 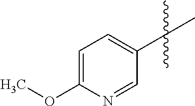 |
| 3 | 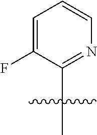 | 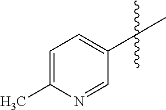 |
| 4 | 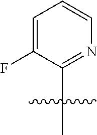 | 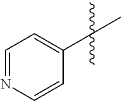 |
| 5 | 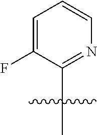 | 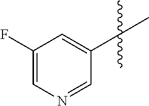 |
| 6 | 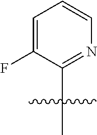 | 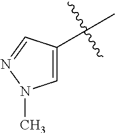 |
| 7 | 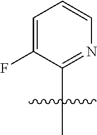 | 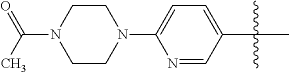 |
| 8 | 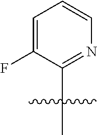 | 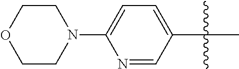 |
| 9 | 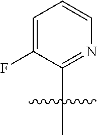 | 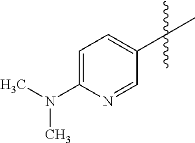 |
| 10 | 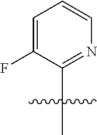 | 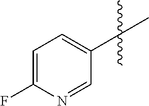 |
| 11 | 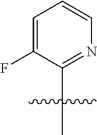 | 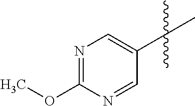 |
| 12 | 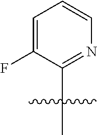 | 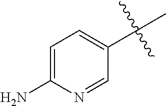 |
| 13 | 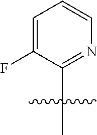 | 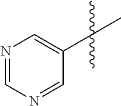 |
| 14 | 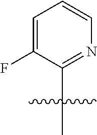 | 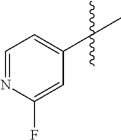 |
| 15 | 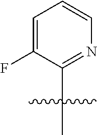 | 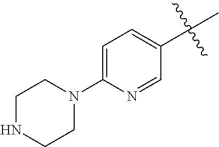 |
| 16 | 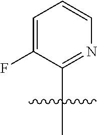 | 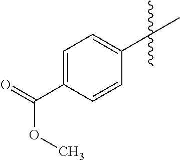 |
| 17 | 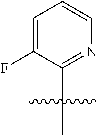 | 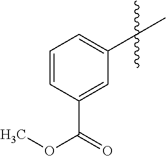 |
| 18 | 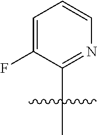 | 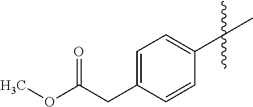 |
| 19 | 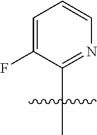 | 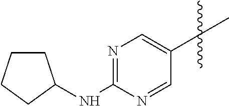 |
| 20 | 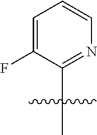 | 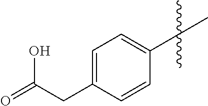 |
| 21 | 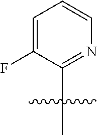 | 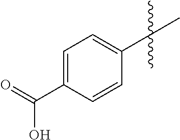 |
| 22 | 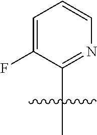 | 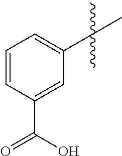 |
| 23 | 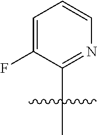 | 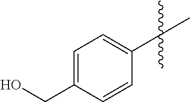 |
| 24 | 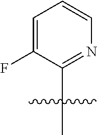 | 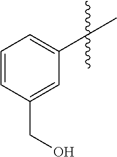 |
| 25 | 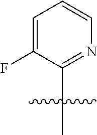 | 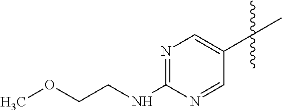 |
| 26 | 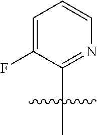 | 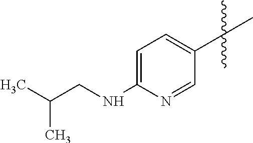 |
| 27 | 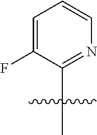 | 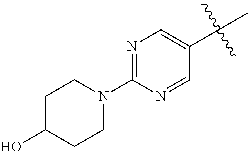 |
| 28 | 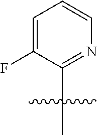 | 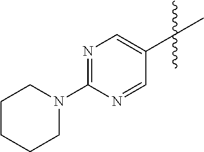 |
| 29 | 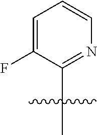 | 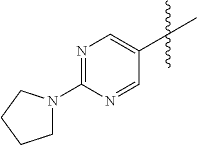 |
| 30 | 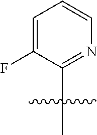 | 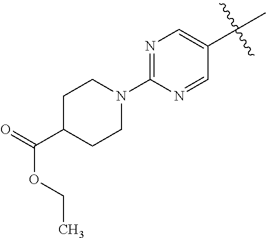 |
| 31 | 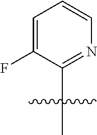 | 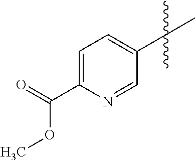 |
| 32 | 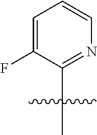 | 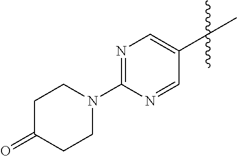 |
| 33 | 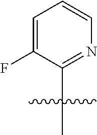 | 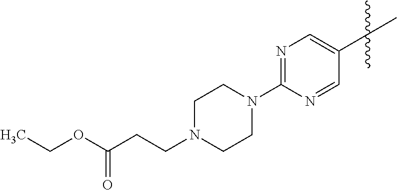 |
| 34 | 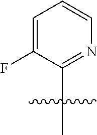 | 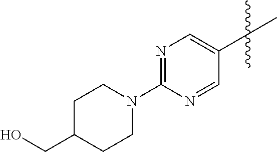 |
| 35 | 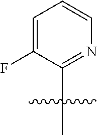 | 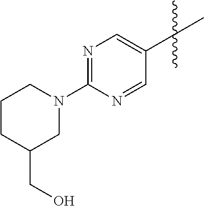 |
| 36 | 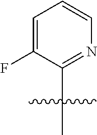 | 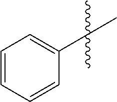 |
| 37 | 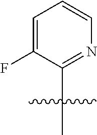 | 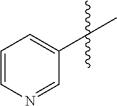 |
| 38 | 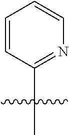 | 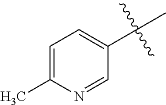 |
| 39 | 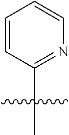 | 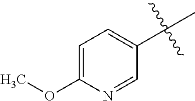 |
| 40 | 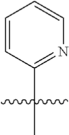 | 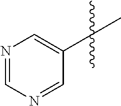 |
| 41 | 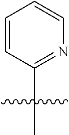 | 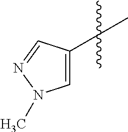 |
| 42 | 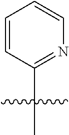 | 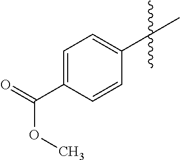 |
| 43 | 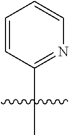 | 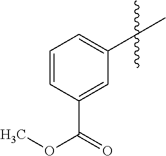 |
| 44 | 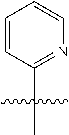 | 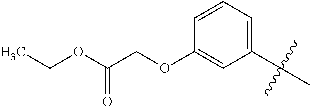 |
| 45 | 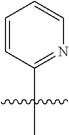 | 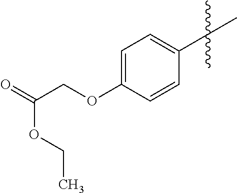 |
| 46 | 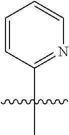 | 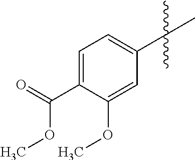 |
| 47 | 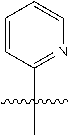 | 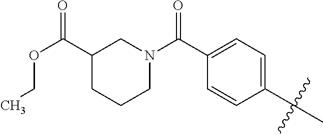 |
| 48 | 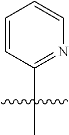 | 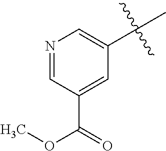 |
| 49 | 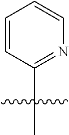 | 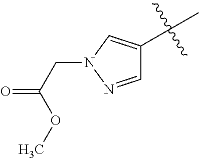 |
| 50 | 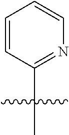 | 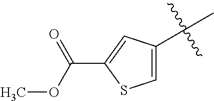 |
| 51 | 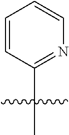 | 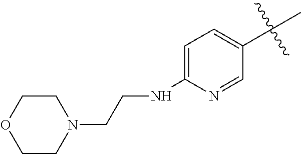 |
| 52 | 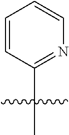 | 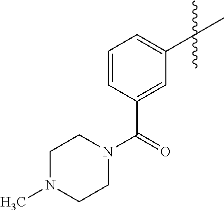 |
| 53 | 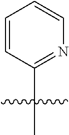 | 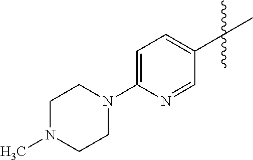 |
| 54 | 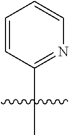 | 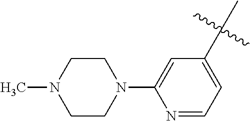 |
| 55 | 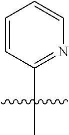 | 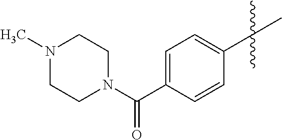 |
| 56 | 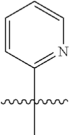 | 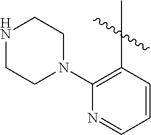 |
| 57 | 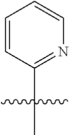 | 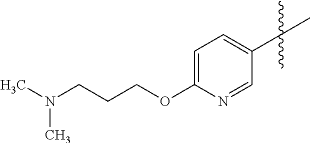 |
| 58 | 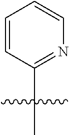 | 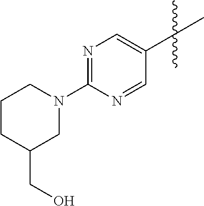 |
| 59 | 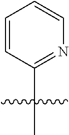 | 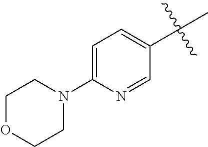 |
| 60 | 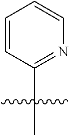 | 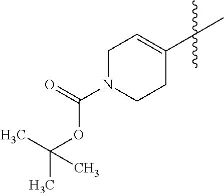 |
| 61 | 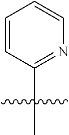 | 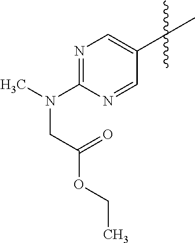 |
| 62 | 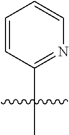 | 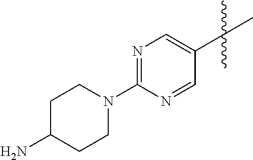 |
| 63 | 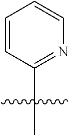 | 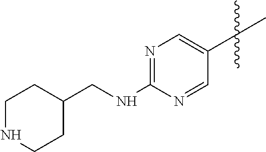 |
| 64 | 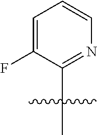 | 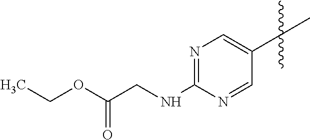 |
| 65 | 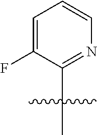 | 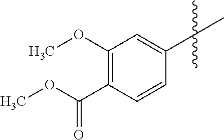 |
| 66 | 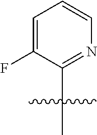 | 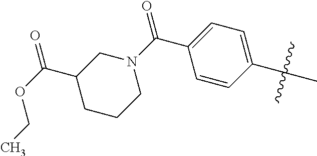 |
| 67 | 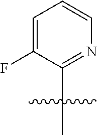 | 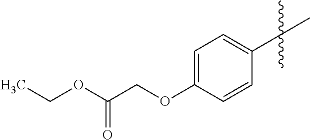 |
| 68 | 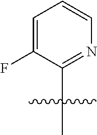 | 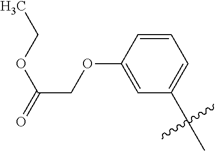 |
| 69 | 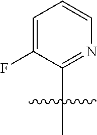 | 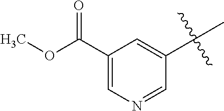 |
| 70 | 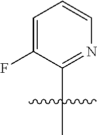 | 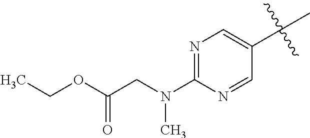 |
| 71 | 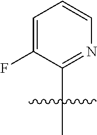 | 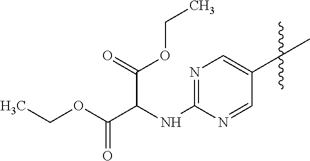 |
| 72 | 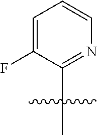 | 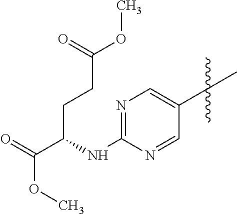 |
| 73 | 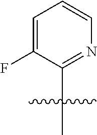 | 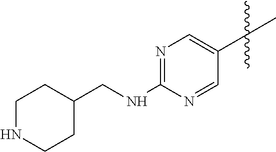 |
| 74 | 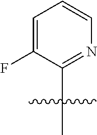 | 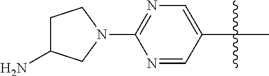 |
| 75 | 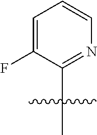 | 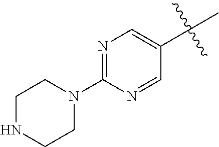 |
| 76 | 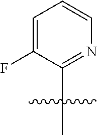 | 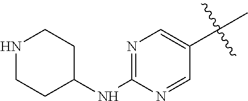 |
| 77 | 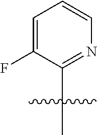 | 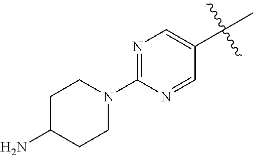 |
| 78 | 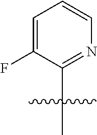 | 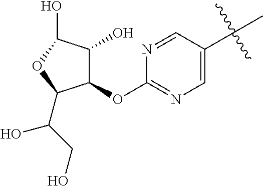 |
| 79 | 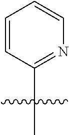 | 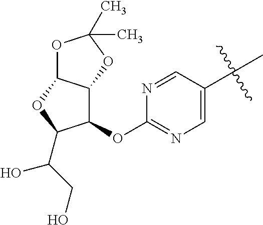 |
| 80 | 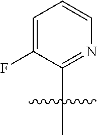 | 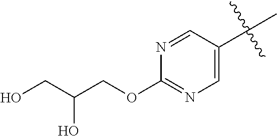 |
| 81 | 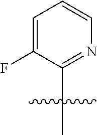 | 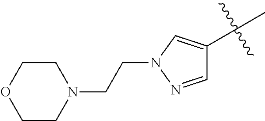 |
| 82 | 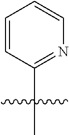 | 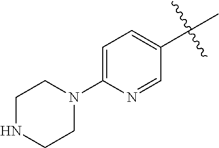 |
| 83 | 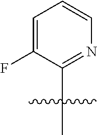 | 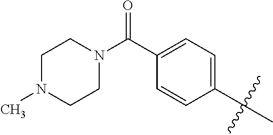 |
| 84 | 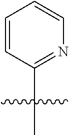 | 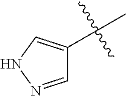 |
| 85 | 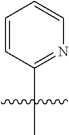 | 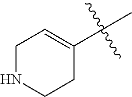 |
| 86 | 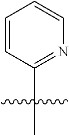 | 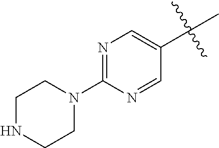 |
| 87 | 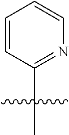 | 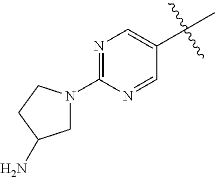 |
| 88 | 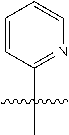 | 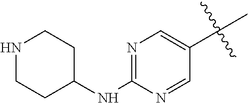 |
| 89 | 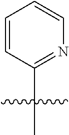 | 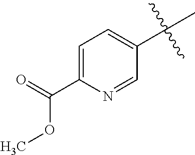 |
| 90 | 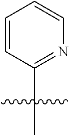 | 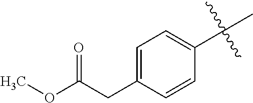 |
| 91 | 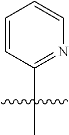 | 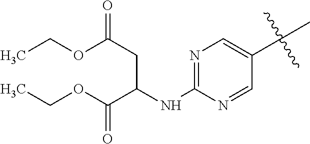 |
| 92 | 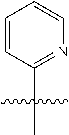 | 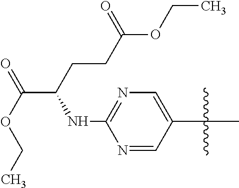 |
| 93 | 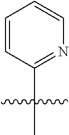 | 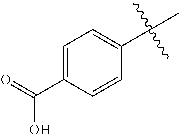 |
| 94 | 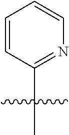 | 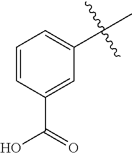 |
| 95 | 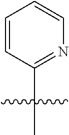 | 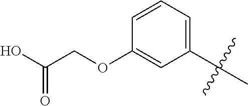 |
| 96 | 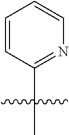 | 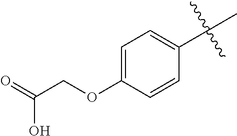 |
| 97 | 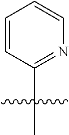 | 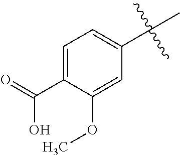 |
| 98 | 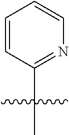 | 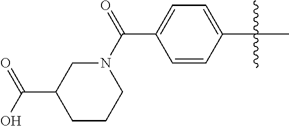 |
| 99 | 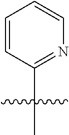 | 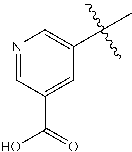 |
| 100 | 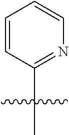 | 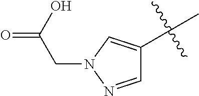 |
| 101 | 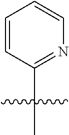 | 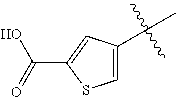 |
| 102 | 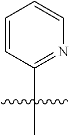 | 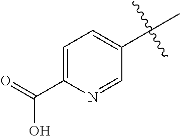 |
| 103 | 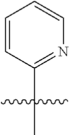 | 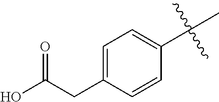 |
| 104 | 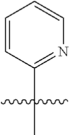 | 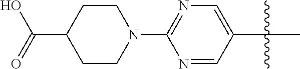 |
| 105 | 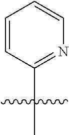 | 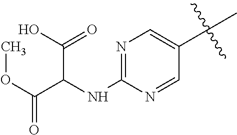 |
| 106 | 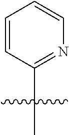 | 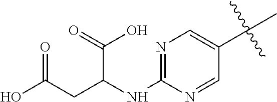 |
| 107 | 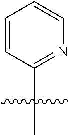 | 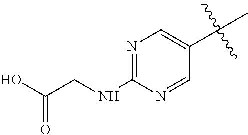 |
| 108 | 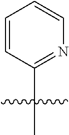 | 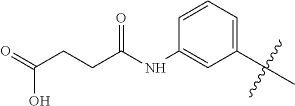 |
| 109 | 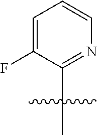 | 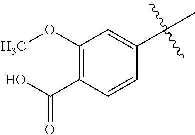 |
| 110 | 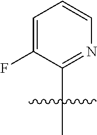 | 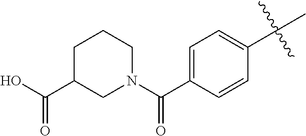 |
| 111 | 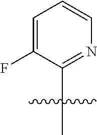 | 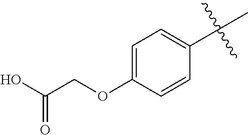 |
| 112 | 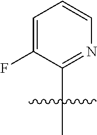 | 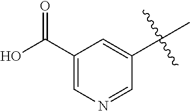 |
| 113 | 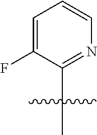 | 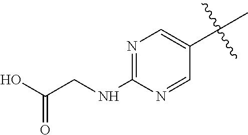 |
| 114 | 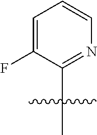 | 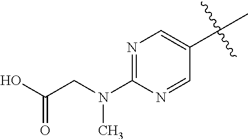 |
| 115 | 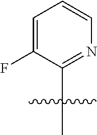 | 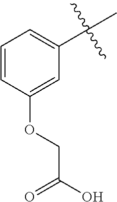 |
| 116 | 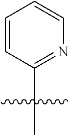 | 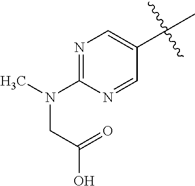 |
| 117 | 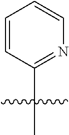 | 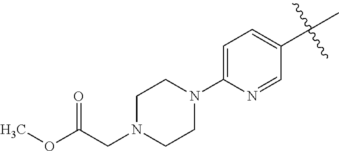 |
| 118 | 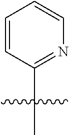 | 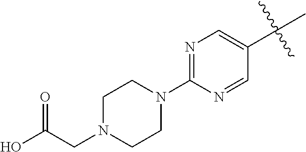 |
| 119 | 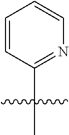 | 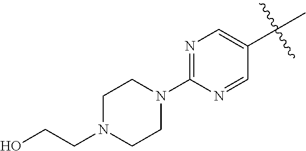 |
| 120 | 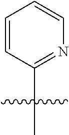 | 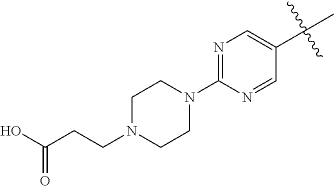 |
| 121 | 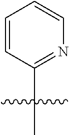 | 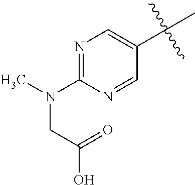 |
| 122 | 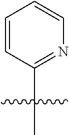 | 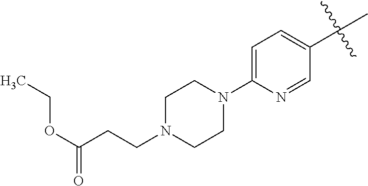 |
| 123 | 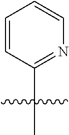 | 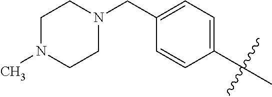 |
| 124 | 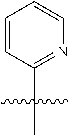 | 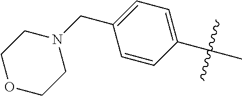 |
| 125 | 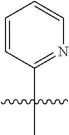 | 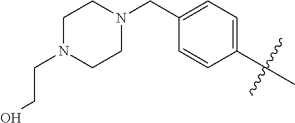 |
| 126 | 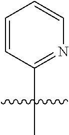 | 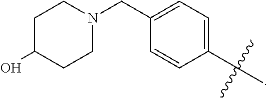 |
| 127 | 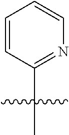 | 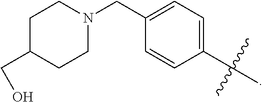 |
| 128 | 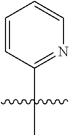 | 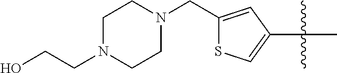 |
| 129 | 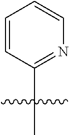 | 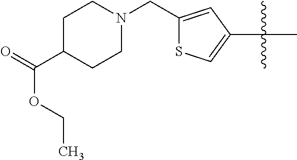 |
| 130 | 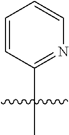 | 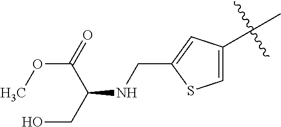 |
| 131 | 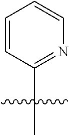 | 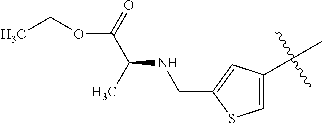 |
| 132 | 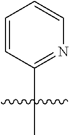 | 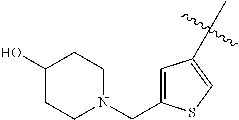 |
| 133 | 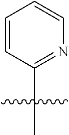 | 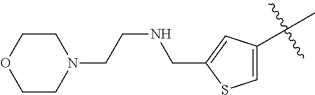 |
| 134 | 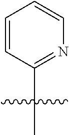 | 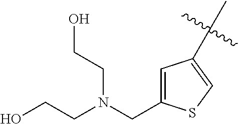 |
| 135 | 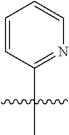 | 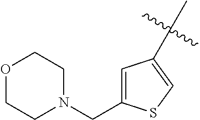 |
| 136 | 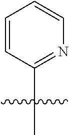 | 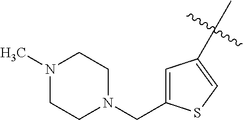 |
| 137 | 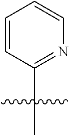 | 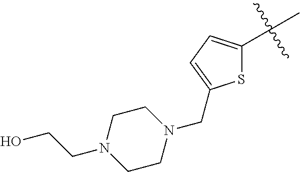 |
| 138 | 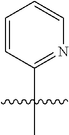 | 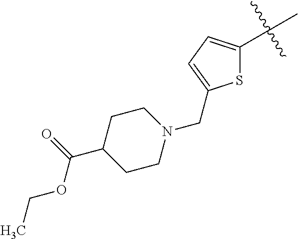 |

The compounds described herein may be administered to an animal for treatment orally, topically, rectally, internasally, or by parenteral route. Pharmaceutical compositions disclosed herein comprise pharmaceutically effective amounts of compounds described herein formulated together with one or more pharmaceutically acceptable carriers, excipients or diluents.

Solid form preparations for oral administration include capsules, tablet, pills, powder, granules, lozenges, troches, cachets and suppositories. For solid form preparations, active compounds can be mixed with one or more inert, pharmaceutically acceptable excipients or carrier, for example, sodium citrate, dicalcium phosphate and/or fillers or extenders (for example, starches, lactose, sucrose, glucose, mannitol, silicic acid or mixtures thereof); binders, for example, carboxymethylcellulose, alginates, gelatins, polyvinylpyrrolidinone, sucrose, acacia or mixtures thereof; disintegrating agents, for example, agar-agar, calcium carbonate, potato starch, alginic acid, certain silicates, sodium carbonate or mixtures thereof; absorption accelerators, for example, quaternary ammonium compounds; wetting agents, for example, cetyl alcohol, glycerol mono stearate or mixtures thereof; adsorbants, for example, Kaolin; lubricants, for example, talc, calcium stearate, magnesium stearate, solid polyethyleneglycol, sodium lauaryl sulfate or mixtures thereof.

Capsules, tablets or pills may also comprise buffering agents.

Tablets, capsules, pills or granules can be prepared using one or more coatings or shells to modulate the release of active ingredients, for example, enteric coatings or other coatings known to one of ordinary skill in the art.

Liquid form preparations for oral administration include pharmaceutically acceptable emulsions, solutions, suspensions, syrups or elixirs. In such liquid form preparations, active compounds can be mixed with water or one or more non-toxic solvents, solubilizing agents or emulsifiers, for example, water, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil, glycerol, fatty acid esters of sorbitan or mixtures thereof. Oral compositions can also include one or more adjuvants, for example, wetting agents, emulsifying agents, suspending agents, sweetening agents, flavoring agents, perfuming agents or mixtures thereof.

Injectable preparations, for example, sterile injections, and aqueous suspensions may be formulated according to methods known to one of ordinary skill in the art, and in particular, using one or more suitable dispersing or wetting and suspending agents. Acceptable vehicles and solvents that may be employed include one or more of water, Ringer's solution, isotonic sodium chloride or mixtures thereof.

Suppositories for rectal administration of the compound of this invention can be prepared by mixing the drug with suitable nonirritating excipients such as coca butter and polyethylene glycols, which are solid at ordinary temperatures but liquid at body temperature an which therefore melt in the rectum and release the drug

Dosage forms for topical or transdermal administration of a compound of the present invention include ointments, pastes, creams, lotions, gels, powders, solutions, sprays, inhalants or patches. Active compounds can be admixed under sterile condition with one or more pharmaceutically acceptable carriers and optionally any preservatives or buffers as may be required. Ophthalmic formulations, eardrops, eye ointments, powders and solutions are also encompassed within the scope of this invention.

Pharmaceutical preparations may be in unit dosage form. In unit dosage form, the preparations can be subdivided into unit doses containing appropriate quantities of active components. Unit dosage forms can be packaged preparations containing discrete capsules, powders, in vials or ampoules, ointments, capsules, sachets, tablets, gels, creams or any combination and number of such packaged forms.

The following examples are set forth to demonstrate general synthetic procedures for the preparation of representative compounds. The examples are provided to illustrate particular aspect of the disclosure and do not limit the scope of the present invention

### EXPERIMENTAL PROCEDURES

Various solvents, for example, dimethylformamide, benzene, tetrahydrofuran, acetonitrile etc were dried using various drying reagents according to procedure as described in the literature.

### Procedure for Synthesis of Intermediate Borate Ester

### Heterocycles

### Example A

### Synthesis of {1-[5-(4,4,5,5-tetramethyl-1,3,2-dioxaborolan-2-yl)pyrimidin-2-yl]piperidin-3-yl}methanol

### Step 1: Synthesis of [1-(5-bromopyrimidin-2-yl)piperidin-3-yl]methanol

To a solution of 2-chloro-5-bromo-pyrimidine (2.0 g, 10.4 mmol) in acetonitrile (15 mL) were added piperidine-3-yl-methanol (2.36 g, 20.8 mmol) and potassium carbonate (7.0 g, 52.0 mmol) at room temperature (˜25° C.). The reaction mixture was heated at about 100° C. for about 6 hours. It was cooled to room temperature (˜25° C.) and concentrated under reduced pressure. The residue obtained was diluted with water and extracted with ethyl acetate (2×100 mL). The organic layer was dried over anhydrous sodium sulfate, filtered and then concentrated under reduced pressure to afford title compound (2.3 g).

### Step 2: Synthesis of {1-[5-(4,4,5,5-tetramethyl-1,3,2-dioxaborolan-2-yl)pyrimidin-2-yl]piperidin-3-yl}methanol

To a solution of [1-(5-bromopyrimidin-2-yl)piperidin-3-yl]methanol (0.5 g, 1.83 mmol) in 1,4 dioxane (25 mL) were added bispinacolatodiborane (0.46 g, 3.3 mmol), potassium acetate (0.53 g, 5.4 mmol) and Pd (dppf)Cl₂ (0.14 g, 0.18 mmol) under argon at room temperature (˜25° C.). The mixture was heated at 80-90° C. for about 6 hours. It was cooled to room temperature (˜25° C.), concentrated under reduced pressure and then purified through filtering column using sintered funnel full of 230-400 mesh size silica gel. It was eluted with 50% ethyl acetate in hexane and concentrated to afford title compound (0.4 g). MS m/e 289.93 (MH⁺).

The following intermediates were synthesized using above synthetic procedure
N-Cyclopentyl-5-(4,4,5,5-tetramethyl-1,3,2-dioxaborolan-2-yl)pyrimidin-2-amine
MS m/e 308.44 (MH⁺).
Ethyl N-[5-(4,4,5,5-tetramethyl-1,3,2-dioxaborolan-2-yl)pyrimidin-2-yl]glycinate
MS m/e 280.43 (MH⁺).
N-(2-Methoxyethyl)-5-(4,4,5,5-tetramethyl-1,3,2-dioxaborolan-2-yl)pyrimidin-2-amine
MS m/e 277.34 (MH⁺).
N-(2-Methylpropyl)-5-(4,4,5,5-tetramethyl-1,3,2-dioxaborolan-2-yl)pyridin-2-amine
MS m/e 306.27 (MH⁺).
1-[5-(4,4,5,5-Tetramethyl-1,3,2-dioxaborolan-2-yl)pyrimidin-2-yl]piperidin-4-ol
MS m/e 290.37 (MH⁺).
2-(Piperidin-1-yl)-5-(4,4,5,5-tetramethyl-1,3,2-dioxaborolan-2-yl)pyrimidine
MS m/e 276.33 (MH⁺).
1-[5-(4,4,5,5-Tetramethyl-1,3,2-dioxaborolan-2-yl)pyrimidin-2-yl]piperidin-4-one
MS m/e 304.34 (MH⁺).
Ethyl 1-[5-(4,4,5,5-tetramethyl-1,3,2-dioxaborolan-2-yl)pyrimidin-2-yl]piperidine-4-carboxylate
MS m/e 362.33 (MH⁺).
tert-Butyl 4-[5-(4,4,5,5-tetramethyl-1,3,2-dioxaborolan-2-yl)pyrimidin-2-yl]piperazine-1-carboxylate
MS m/e 391.37 (MH⁺).
{1-[5-(4,4,5,5-Tetramethyl-1,3,2-dioxaborolan-2-yl)pyrimidin-2-yl]piperidin-4-yl}methanol
MS m/e 320.33 (MH⁺).
Diethyl N-[5-(4,4,5,5-tetramethyl-1,3,2-dioxaborolan-2-yl)pyrimidin-2-yl]aspartate
MS m/e 394.28 (MH⁺).
Dimethyl {[5-(4,4,5,5-tetramethyl-1,3,2-dioxaborolan-2-yl)pyrimidin-2-yl]amino}propanedioate
MS m/e 380.30 (MH⁺).
tert-Butyl {1-[5-(4,4,5,5-tetramethyl-1,3,2-dioxaborolan-2-yl)pyrimidin-2-yl]pyrrolidin-3-yl}carbamate
MS m/e 391.37 (MH⁺).
tert-Butyl 4-{[5-(4,4,5,5-tetramethyl-1,3,2-dioxaborolan-2-yl)pyrimidin-2-yl]amino}piperidine-1-carboxylate
MS m/e 405.35 (MH⁺).
Ethyl N-methyl-N-[5-(4,4,5,5-tetramethyl-1,3,2-dioxaborolan-2-yl)pyrimidin-2-yl]glycinate
MS m/e 322.28 (MH⁺).
tert-Butyl {1-[5-(4,4,5,5-tetramethyl-1,3,2-dioxaborolan-2-yl)pyrimidin-2-yl]piperidin-4-yl}carbamate
MS m/e 405.31 (MH⁺).
tert-Butyl 4-({[5-(4,4,5,5-tetramethyl-1,3,2-dioxaborolan-2-yl)pyrimidin-2-yl]amino}methyl)piperidine-1-carboxylate
MS m/e 419.31 (MH⁺).
Dimethyl N-[5-(4,4,5,5-tetramethyl-1,3,2-dioxaborolan-2-yl)pyrimidin-2-yl]-L-glutamate
MS m/e 380.30 (MH⁺).

### Example B

### Synthesis of 2-{[(3aR,5R,6S,6aR)-5-(2,2-dimethyl-1,3-dioxolan-4-yl)-2,2-dimethyltetrahydro-furo[2,3-d][1,3]dioxol-6-yl]oxy}-5-(4,4,5,5-tetramethyl-1,3,2-dioxaborolan-2-yl)pyrimidine

### Step 1: Synthesis of 5-bromo-2-{[(3aR,5R,6S,6aR)-5-(2,2-dimethyl-1,3-dioxolan-4-yl)-2,2-dimethyl-tetrahydrofuro[2,3-d][1,3]dioxol-6-yl]oxy}pyrimidine

To a solution of (3aR,5S,6S,6aR)-5-(2,2-dimethyl-1,3-dioxolan-4-yl)-2,2-dimethyltetrahydrofuro[2,3-d][1,3]dioxol-6-ol (1.6 g, 6.2 mmol) in dimethylformamide (15 mL) was added sodium hydride (0.23 g, 13.6 mmol) at about 0° C. and the reaction mixture was warmed to room temperature (−25° C.) and stirred for about 30 mins. It was then cooled to about 0° C. and a solution of 2-chloro-5-bromo pyrimidine (1 g, 0.0051 mmol) in dimethylformamide was added to the reaction at the rate to keep the internal temperature below 5° C. The reaction mixture was then warmed to room temperature (−25° C.) and stirred for about 3 hours and then poured into cold water. The aqueous layer was then extracted with 2×50 mL of ethyl acetate. The combined organic layer was washed with water, dried over anhydrous sodium sulfate, filtered and then concentrated under reduced pressure to afford title compound (0.85 g).

### Step 2: Synthesis of 2-{[(3aR,5R,6S,6aR)-5-(2,2-dimethyl-1,3-dioxolan-4-yl)-2,2-dimethyltetrahydro-furo[2,3-d][1,3]dioxol-6-yl]oxy}-5-(4,4,5,5-tetramethyl-1,3,2-dioxaborolan-2-yl)pyrimidine

To a solution of 5-bromo-2-{[(3aR,5R,6S,6aR)-5-(2,2-dimethyl-1,3-dioxolan-4-yl)-2,2-dimethyl-tetrahydrofuro[2,3-d][1,3]dioxol-6-yl]oxy}pyrimidine (0.7 g, 1.6 mmol) in 1,4 dioxane (25 mL) were added bispinacolatodiborane (0.57 g, 2.0 mmol), potassium acetate (0.49 g, 5.0 mmol) and Pd (dppf)Cl₂ (0.13 g, 0.16 mmol) under argon at room temperature (˜25° C.). The mixture was heated at 80-90° C. for about 6 hours. It was cooled to room temperature (˜25° C.)., concentrated under reduced pressure and then purified through filtering column using sintered funnel full of 230-400 mesh size silica gel. It was eluted with 50% ethyl acetate in hexane and concentrated to afford title compound (0.4 g).

MS m/e 465.25 (MH⁺)

The following intermediate was synthesized using the same synthetic procedure as above 2-[(2,2-dimethyl-1,3-dioxolan-4-yl)methoxy]-5-(4,4,5,5-tetramethyl-1,3,2-dioxaborolan-2-yl)pyrimidine

MS m/e 337.29 (MH⁺)

### Example C

### Synthesis of methyl 5-(4,4,5,5-tetramethyl-1,3,2-dioxaborolan-2-yl)pyridine-2-carboxylate

### Step 1: Synthesis of 5-bromo-pyridine-2-carbaldehyde

To a solution of 2-Iodo-5-bromo-pyridine (25.0 g, 104.0 mmol) in tetrahydrofuran (150 mL) were added isopropyl magnesium chloride (58 mL, 112.0 mmol) at −15° C. to −10° C. at an interval of about 1 hour. The reaction mixture was further stirred at −15° C. to 0° C. for about 1 hour. Freshly distilled anhydrous dimethylformamide (12 mL, 156.0 mmol) was added to the reaction mixture while keeping the temperature of the reaction mixture below 0° C.

After stirring at this temperature for about 30 min, the reaction mixture was allowed to warm to room temperature over 1 hour. the reaction mixture was then cooled to about 0° C. and 50 mL of 2N HCl aqueous solution was added at a rate to keep the internal temperature below 25° C. The mixture was stirred for 30 mins The pH was adjusted to pH 6-7 by adding 2N NaOH aqueous solution. The aqueous layer was then extracted with 2×250 mL of dichloromethane The combined organic layer was washed with water, dried over anhydrous sodium sulfate, filtered and then concentrated under reduced pressure to afford title compound (17 g)

### Step 2: Synthesis of 5-bromo-pyridine-2-carboxylic acid

To a solution of 5-bromo-pyridine-2-carbaldehyde (1.5 g, 8.0 mmol) in water (20 mL) was added sodium carbonate (0.42 g, 4.0 mmol) and the reaction mixture was cooled to about 10° C. Aqueous potassium permanganate solution (1.9 g, 12.0 mmol) was added to the reaction mixture while keeping the temperature at about 10° C. The reaction mixture was then stirred at room temperature for four hours. The pH was adjusted to pH 2-4 by adding 2N HCl solution. It was then extracted with ethylacetate (3×25 mL). The combined organic layer was washed with water, dried over anhydrous sodium sulfate, filtered and then concentrated under reduced pressure to afford title compound (1.4 g).

### Step 3: Synthesis of 5-bromo-pyridine-2-carboxylic acid methyl ester

To a solution of 5-bromo-pyridine-2-carboxylic acid (1.2 g, 5.0 mmol) in methanol (20 mL) was added catalytic amount of conc. sulfuric acid (0.5 mL) while keeping the temperature of reaction mixture between 10° C. to 15° C. The reaction mixture was then refluxed for about 5 hours and then the solvent was evaporated. The oily residue was then poured into cold water (25 mL) and pH was adjusted to pH 7-8 by adding 2N NaOH aqueous solution. The aqueous layer was then extracted with 2×250 mL of dichloromethane. The combined organic layer was washed with water, dried over anhydrous sodium sulfate, filtered and then concentrated under reduced pressure to afford title compound (1.2 g).

### Step 4: Synthesis of methyl 5-(4,4,5,5-tetramethyl-1,3,2-dioxaborolan-2-yl)pyridine-2-carboxylate

To a solution of 5-bromo-pyridine-2-carboxylic acid methyl ester (0.5 g, 2.3 mmol) in 1,4 dioxane (25 mL) were added bispinacolatodiborane (1.05 g, 4.1 mmol), potassium acetate (1.13 g, 11.5 mmol) and Pd (dppf)Cl₂ (0.28 g, 0.34 mmol) under argon at room temperature. The mixture was heated at 80-90° C. for about 6 hours. It was cooled to room temperature, concentrated under reduced pressure and then purified through filtering column using sintered funnel full of 230-400 mesh size silica gel. It was eluted with 50% ethylacetate in hexane and concentrated to afford title compound (0.23 g).

MS m/e 0.264.27. (MH⁺)

The following intermediates were synthesized using synthetic procedure as given above
Methyl[4-(4,4,5,5-tetramethyl-1,3,2-dioxaborolan-2-yl)phenyl]acetate
MS m/e 291.32 (MH⁺)
Methyl 4-(4,4,5,5-tetramethyl-1,3,2-dioxaborolan-2-yl)thiophene-2-carboxylate
MS m/e 269.05 (MH⁺).

### Example 1

### Synthesis of 1-ethyl-3-[4-fluoro-7-(3-fluoropyridin-2-yl)-5-(pyridin-3-yl)-1,3-benzothiazol-2-yl]urea (Compound No. 1)

### Step 1: Synthesis of N-(2-bromo-5-fluorophenyl)-2,2-dimethylpropanamide

To a solution of 2-bromo-5-fluoroaniline (15.0 g, 85.7 mmol) in dichloromethane (120 mL) were added triethylamine (11.25 g, 110.0 mmol) followed by pivaloyl chloride (11.3 g, 92.0 mmol) drop-wise at 0° C. and then stirred at 25-30° C. for about 4 hours. The reaction mixture was diluted with dichloromethane and washed with saturated solution of sodium bicarbonate followed by water. The organic layer was dried over anhydrous sodium sulfate, filtered and then concentrated to give the crude residue, which was purified through column chromatography using silica gel 230-400 mesh size and eluted with 5% ethyl acetate in hexane to yield the title compound (20 g)

### Step 2: Synthesis of N-[5-fluoro-2-(4,4,5,5-tetramethyl-1,3,2-dioxaborolan-2-yl)phenyl]-2,2-dimethylpropanamide

To a solution of N-(2-bromo-5-fluorophenyl)-2,2-dimethylpropanamide (0.5 g, 1.93 mmol) in dioxane (20 mL) were added (bispinacolato)diboron (0.97 g, 3.86 mmol), [bis-(diphenyl-phosphino)ferrocene palladium II dichloride [Pd (dppf)Cl₂] (0.158 g, 0.197 mmol) and potassium acetate (0.95 g, 9.65 mmol) under argon at 25-30° C. The reaction mixture was heated at about 100-105° C. for a period of about 4 hours. The reaction mixture was cooled to 25-30° C., filtered and washed with dichloromethane. The combined filtrates were concentrated under reduced pressure. The residue obtained was diluted with water and extracted with ethyl acetate. The organic layer was dried over anhydrous sodium sulfate, filtered and concentrated under reduced pressure. The crude residue was purified through column chromatography using silica gel 230-400 mesh and eluted with 5-10% ethyl acetate in hexane to yield the title compound (0.6 g)

MS m/e 322.72 (MH⁺).

### Step 3: Synthesis of N-[5-fluoro-2-(3-fluoropyridin-2-yl)phenyl]-2,2-dimethylpropanamide

To a solution of N-[5-fluoro-2-(4,4,5,5-tetramethyl-1,3,2-dioxaborolan-2-yl)phenyl]-2,2-dimethylpropanamide (8.0 g, 24.9 mmol) in acetonitrile:H₂O (96 mL:16 mL), was added 2-chloro-3-fluoropyridine (4.57 g, 34.0 mmol), Pd(PPh₃)₄ (2.87 g, 2.4 mmol) and potassium carbonate (10.39 g, 74.0 mmol) in under argon at 25-30° C. The reaction mixture was heated at 80-85° C. over a period of about 8 hours. The mixture was cooled to 25-30° C. and diluted with water, extracted with ethyl acetate. The organic layer was dried over anhydrous sodium sulfate, filtered and then concentrated under reduced pressure. The crude product obtained was purified through column chromatography using silica gel 230-400 mesh size and eluted with gradient of 5-10% ethyl acetate in hexane provided the title compound (3.0 g).

MS m/e 291.59 (MH⁺).

### Step 4: Synthesis of N-[4-bromo-5-fluoro-2-(3-fluoropyridin-2-yl)phenyl]-2,2-dimethylpropanamide

To a solution of N-[5-fluoro-2-(3-fluoropyridin-2-yl)phenyl]-2,2-dimethylpropanamide (3.5 g, 12.0 mmol) in acetic acid (45 mL) was added bromine (1.8 mL, 36.0 mmol) as a solution in 8 mL of acetic acid over a period of about 1 hour. The reaction mixture was stirred at 25-30° C. for about 5 hours and then poured over ice, diluted with 1N sodium thiosulfate (100 mL) or sodium bisulfite and stirred for 30 minute, then filtered and washed with water. The resulting solid was dried under vacuum to afford the title compound (3.6 g).

MS m/e 369.49 (MH⁺).

### Step 5: Synthesis of N-[4-bromo-3-fluoro-6-(3-fluoropyridin-2-yl)-2-nitrophenyl]-2,2-dimethylpropanamide

To a solution of N-[4-bromo-5-fluoro-2-(3-fluoropyridin-2-yl)phenyl]-2,2-dimethyl-propanamide (3.0 g, 10.8 mmol) in trifluoroacetic acid (59 mL) and trifluoroacetic anhydride (15 mL) at 0° C., trifluoroacetic acid solution of 90% fuming nitric acid (1.45 mL, 32.4 mmol in 18 mL trifluoroacetic acid) was added over about 45 minute period. The reaction mixture was then stirred at 0° C. for 4-5 hours. The reaction mixture was slowly poured in to ice water and stirred for about 1 hour and then extracted with ethyl acetate. The organic layer was dried over anhydrous sodium sulfate, filtered and then concentrated under vacuum. The semisolid residue obtained was purified through column chromatography using silica gel 230-400 mesh size and eluted with 1% methanol in dichloromethane to provide title compound (1.0 g)

MS m/e 414.52 (MH⁺).

### Step 6: Synthesis of 4-bromo-3-fluoro-6-(3-fluoropyridin-2-yl)-2-nitroaniline

A solution of N-[4-bromo-3-fluoro-6-(3-fluoropyridin-2-yl)-2-nitrophenyl]-2,2-dimethyl-propanamide (1.0 g, 2.4 mmol) in 70% aq. sulphuric acid (3.0 mL) was refluxed at 80-85° C. for a period of about 2 hours. The reaction mixture was cool to 25-30° C. and then poured over crushed ice and stirred for 30 minute. The resultant mixture was basified with 20% aq. Sodium hydroxide solution up to pH 10. The resultant solid was filtered, washed with water and then dried under vacuum to afford the title compound (0.65 g).

MS m/e 330.44 (MH⁺).

### Step 7: Synthesis of 2-(5-bromo-4-fluoro-2-iodo-3-nitrophenyl)-3-fluoropyridine

To a solution of 4-bromo-3-fluoro-6-(3-fluoropyridin-2-yl)-2-nitroaniline (0.55 g, 1.67 mmol) in chloroform (15 mL) were added iodine (2.4 g, 9.36 mmol) and isoamylnitrile (3.34 mmol) in portion wise and then refluxed for about 5 hours. The reaction mixture was cool to 25-30° C. diluted with 10% sodium bisulphite solution and extracted with dichloromethane. The organic layer was dried over anhydrous sodium sulfate, filtered and concentrated under reduced pressure. The crude residue was purified through column chromatography using silica gel 230-400 mesh size and eluted with gradient of 5-10% ethyl acetate in hexane provided the title compound (0.3 g).

MS m/e 441.41 (MH⁺).

### Step 8: Synthesis 3-bromo-2-fluoro-5-(3-fluoropyridin-2-yl)-6-iodoaniline

To a solution of 2-(5-bromo-4-fluoro-2-iodo-3-nitrophenyl)-3-fluoropyridine (0.27 g, 0.568 mmol) in ethanol and water (6 mL: 6 mL) was added Fe powder (0.269 g, 4.82 mmol), and conc. hydrochloric acid (0.058 mL). The reaction mixtures were refluxed for about 2 hours and then cool to 25-30° C., filtered through celite pad and washed with ethyl acetate. The filtrate was concentrated under reduced pressure and then aqueous layer was extracted with ethyl acetate. The organic layer was dried over anhydrous sodium sulfate, filtered and concentrated to afford title compound (0.255 g) MS m/e 411.53 (MH⁺).

### Step 9: Synthesis of N-[5-bromo-4-fluoro-7-(3-fluoropyridin-2-yl)-1,3-benzothiazol-2-yl]benzamide)

To a solution of 3-bromo-2-fluoro-5-(3-fluoropyridin-2-yl)-6-iodoaniline (0.25 g, 0.609 mmol) in acetone (5 mL), benzoylisothiocyanate (0.109 g, 0.67 mmol) was added drop-wise over a period of about 20 minutes. The reaction mixture was stirred at 25-30° C. for about 10 hours. The solvent was removed under reduced pressure and the residue obtained was triturated with diethyl ether. The solid separated was filtered, dried under vacuum to afford title compound (0.175 g).

MS m/e 446.59 (MH⁺).

### Step 10: Synthesis of 5-bromo-4-fluoro-7-(3-fluoropyridin-2-yl)-1,3-benzothiazol-2-amine

To a solution of N-[5-bromo-4-fluoro-7-(3-fluoropyridin-2-yl)-1,3-benzothiazol-2-yl]benzamide (0.2 g) in methanol (3 mL) was added 2 N aq. sodium hydroxide solution (3 mL). The reaction mixture was refluxed at 100-110° C. for about 18 hours. The solvent was removed under reduced pressure and then poured in to water. The solid precipitate was filtered, washed with water and then dried under vacuum to afford title compound (0.13 g)

MS m/e 342.47 (MH⁺).

### Step 11: Synthesis of S-methyl[5-bromo-4-fluoro-7-(3-fluoropyridin-2-yl)-1,3-benzothiazol-2-yl]carbamothioate

To a solution of 5-bromo-4-fluoro-7-(3-fluoropyridin-2-yl)-1,3-benzothiazol-2-amine (0.125 g, 0.366 mmol) in pyridine (4 mL) was added methyl(chloro)thioformate (0.082 g, 0.73 mmol) drop-wise under argon and then heated at 50-55° C. for about 12 hours. The reaction mixture was cooled to 25-30° C. and slowly poured in to ice. The solid precipitate was filtered, washed with water and then dried under vacuum to afford title compound (0.15 g), which is taken as such for next step.

### Step 12: Synthesis of 1-[5-bromo-4-fluoro-7-(3-fluoropyridin-2-yl)-1,3-benzothiazol-2-yl]-3-ethylurea

To a solution of S-methyl[5-bromo-4-fluoro-7-(3-fluoropyridin-2-yl)-1,3-benzothiazol-2-yl]carbamothioate (145 g, 0.342 mmol) in ethanol (4 mL) was added ethylamine (1.8 mL, 2.0 m tetrahydrofuran solution) and then refluxed for about 18 hours. The reaction mixture concentrated under reduced pressure. The residue obtained was triturated with water, filtered and dried under vacuum. The solid residue was again triturated with diethyl ether, filtered and dried under vacuum to afford title compound (0.125 g).

MS m/e 413.54 (MH⁺).

### Step 13: Synthesis of 1-ethyl-3-[4-fluoro-7-(3-fluoropyridin-2-yl)-5-(pyridin-3-yl)-1,3-benzothiazol-2-yl]urea

To a solution of 1-[5-bromo-4-fluoro-7-(3-fluoropyridin-2-yl)-1,3-benzothiazol-2-yl]-3-ethylurea (0.025 g, 0.060 mmol) in acetonitrile:water 9:1 (3 mL) were added pyridine-3-boronic acid (0.009 g, 0.0728 mmol), potassium carbonate (0.025 g, 0.182 mmol) and Pd(dppf)Cl₂ (0.005 g, 0.0060 mmol). The reaction mixture was run in microwave at about 110° C. for about 25 minutes. The reaction mixture was cooled to 25-30° C., diluted with ethyl acetate and then washed with water followed by brine solution. The organic layer was dried over anhydrous sodium sulfate, filtered and concentrated under reduced pressure. The residual solid obtained was purified through column chromatography using silica gel 230-400 mesh size and eluted with gradient of 1-5% methanol in dichloromethane provided the title compound (0.016 g).

¹H NMR (400 MHz, DMSO-d₆) δ: 11.15 (bs, 1H), 8.99 (s, 1H), 8.82-8.77 (m, 2H), 8.22-8.06 (m, 3H), 7.71-7.70 (m, 2H), 6.85 (bs, 1H) 3.32 (q, 2H), 1.22 (t, 3H)

MS m/e 412.67 (MH⁺).

The following compounds were prepared employing procedures as provided in Examples 1 described above:
1-Ethyl-3-[4-fluoro-7-(3-fluoropyridin-2-yl)-5-(6-methyoxypyridin-3-yl)-1,3-benzothiazol-2-yl]urea (Compound No. 2)
MS m/e 442.72 (MH⁺).
1-Ethyl-3-[4-fluoro-7-(3-fluoropyridin-2-yl)-5-(6-methylpyridin-3-yl)-1,3-benzothiazol-2-yl]urea (Compound No. 3),
MS m/e 426.71 (MH⁺).
1-Ethyl-3-[4-fluoro-7-(3-fluoropyridin-2-yl)-5-(pyridin-4-yl)-1,3-benzothiazol-2-yl]urea (Compound No. 4)
MS m/e 412.74 (MH⁺)
1-Ethyl-3-[4-fluoro-7-(3-fluoropyridin-2-yl)-5-(5-fluoropyridin-3-yl)-1,3-benzothiazol-2-yl]urea (Compound No. 5)
MS m/e 430.67 (MH⁺)
1-Ethyl-3-[4-fluoro-7-(3-fluoropyridin-2-yl)-5-(1-methyl-1H-pyrazol-4-yl)-1,3-benzothiazol-2-yl]urea (Compound No. 6)
MS m/e 415.69 (MH⁺)
1-{5-[6-(4-Acetylpiperazin-1-yl)pyridin-3-yl]-4-fluoro-7-(3-fluoropyridin-2-yl)-1,3-benzothiazol-2-yl}-3-ethylurea (Compound No. 7)
MS m/e 538.10 (MH⁺)
1-Ethyl-3-{4-fluoro-7-(3-fluoropyridin-2-yl)-5-[6-(morpholin-4-yl)pyridin-3-yl]-1,3-benzothiazol-2-yl}urea (Compound No. 8)
MS m/e 497.92 (MH⁺)
1-{5-[6-(Dimethylamino)pyridin-3-yl]-4-fluoro-7-(3-fluoropyridin-2-yl)-1,3-benzothiazol-2-yl}-3-ethylurea (Compound No. 9)
MS m/e 455.80 (MH⁺)
1-Ethyl-3-[4-fluoro-7-(3-fluoropyridin-2-yl)-5-(6-fluoropyridin-3-yl)-1,3-benzothiazol-2-yl]urea (Compound No. 10)
MS m/e 430.74 (MH⁺)
1-Ethyl-3-[4-fluoro-7-(3-fluoropyridin-2-yl)-5-(2-methoxypyrimidin-5-yl)-1,3-benzothiazol-2-yl]urea (Compound No. 11)
MS m/e 443.77 (MH⁺)
1-[5-(6-Aminopyridin-3-yl)-4-fluoro-7-(3-fluoropyridin-2-yl)-1,3-benzothiazol-2-yl]-3-ethylurea (Compound No. 12)
MS m/e 427.25 (MH⁺)
1-Ethyl-3-[4-fluoro-7-(3-fluoropyridin-2-yl)-5-(pyrimidin-5-yl)-1,3-benzothiazol-2-yl]urea (Compound No. 13)
MS m/e 413.74 (MH⁺)
1-Ethyl-3-[4-fluoro-5-(2-fluoropyridin-4-yl)-7-(3-fluoropyridin-2-yl)-1,3-benzothiazol-2-yl]urea (Compound No. 14)
MS m/e 430.74 (MH⁺)
1-Ethyl-3-{4-fluoro-7-(3-fluoropyridin-2-yl)-5-[6-(piperazin-1-yl)pyridin-3-yl]-1,3-benzothiazol-2-yl}urea (Compound No. 15)
MS m/e 495.99 (MH⁺)
Methyl 4-{2-[(ethylcarbamoyl)amino]-4-fluoro-7-(3-fluoropyridin-2-yl)-1,3-benzothiazol-5-yl}benzoate (Compound No. 16)
MS m/c 469.27 (MH⁺)
Methyl 3-{2-[(ethylcarbamoyl)amino]-4-fluoro-7-(3-fluoropyridin-2-yl)-1,3-benzothiazol-5-yl}benzoate (Compound No. 17)
MS m/e 469.27 (MH⁺)
Methyl (4-{2-[(ethylcarbamoyl)amino]-4-fluoro-7-(3-fluoropyridin-2-yl)-1,3-benzothiazol-5-yl}phenyl)acetate (Compound No. 18)
MS m/e 483.25 (MH⁺)
1-{5-[2-(Cyclopentylamino)pyrimidin-5-yl]-4-fluoro-7-(3-fluoropyridin-2-yl)-1,3-benzothiazol-2-yl}-3-ethylurea (Compound No. 19)
MS m/e 496.29 (MH⁺)
(4-{2-[(Ethylcarbamoyl)amino]-4-fluoro-7-(3-fluoropyridin-2-yl)-1,3-benzothiazol-5-yl}phenyl)acetic acid (Compound No. 20)
MS m/e 469.13 (MIT)
4-{2-[(Ethylcarbamoyl)amino]-4-fluoro-7-(3-fluoropyridin-2-yl)-1,3-benzothiazol-5-yl}benzoic acid (Compound No. 21)
MS m/e 455.15 (MH⁺)
3-{2-[(Ethylcarbamoyl)amino]-4-fluoro-7-(3-fluoropyridin-2-yl)-1,3-benzothiazol-5-yl}benzoic acid (Compound No. 22)
MS m/e 455.15 (MH⁺)
1-Ethyl-3-{4-fluoro-7-(3-fluoropyridin-2-yl)-5-[4-(hydroxymethyl)phenyl]-benzothiazol-2-yl}urea (Compound No. 23)
MS m/e 441.17 (MH⁺)
1-Ethyl-3-{4-fluoro-7-(3-fluoropyridin-2-yl)-5-[3-(hydroxymethyl)phenyl]-1,3-benzothiazol-2-yl}urea (Compound No. 24)
MS m/e 441.17 (MH⁺)
1-Ethyl-3-[4-fluoro-7-(3-fluoropyridin-2-yl)-5-{2-[(2-methoxyethyl)amino]pyrimidin-5-yl}-1,3-benzothiazol-2-yl]urea (Compound No. 25)
MS m/e 486.21 (MH⁺)
1-Ethyl-3-[4-fluoro-7-(3-fluoropyridin-2-yl)-5-{6-[(2-methylpropyl)amino]pyridin-3-yl}-1,3-benzothiazol-2-yl]urea (Compound No. 26)
MS m/e 483.25 (MH⁺)
1-Ethyl-3-{4-fluoro-7-(3-fluoropyridin-2-yl)-5-[2-(4-hydroxypiperidin-1-yl)pyrimidin-5-yl]-1,3-benzothiazol-2-yl}urea (Compound No. 27)
MS m/e 512.29 (MH⁺)
1-Ethyl-3-{4-fluoro-7-(3-fluoropyridin-2-yl)-5-[2-(piperidin-1-yl)pyrimidin-5-yl]-1,3-benzothiazol-2-yl}urea (Compound No. 28)
MS m/e 496.32 (MH⁺)
1-Ethyl-3-{4-fluoro-7-(3-fluoropyridin-2-yl)-5-[2-(pyrrolidin-1-yl)pyrimidin-5-yl]-1,3-benzothiazol-2-yl}urea (Compound No. 29)
MS m/e 482.29 (MH⁺)
Ethyl 1-(5-{2-[(ethylcarbamoyl)amino]-4-fluoro-7-(3-fluoropyridin-2-yl)-1,3-benzothiazol-5-yl}pyrimidin-2-yl)piperidine-4-carboxylate (Compound No. 30)
MS m/e 568.27 (MH⁺)
Methyl 5-{2-[(ethylcarbamoyl)amino]-4-fluoro-7-(3-fluoropyridin-2-yl)-1,3-benzothiazol-5-yl}pyridine-2-carboxylate (Compound No. 31)
MS m/e 470.20 (MH⁺)
1-Ethyl-3-{4-fluoro-7-(3-fluoropyridin-2-yl)-5-[2-(4-oxopiperidin-1-yl)pyrimidin-5-yl]-1,3-benzothiazol-2-yl}urea (Compound No. 32)
MS m/e 510.19 (MH⁺)
Ethyl 3-[4-(5-{2-[(ethylcarbamoyl)amino]-4-fluoro-7-(3-fluoropyridin-2-yl)-1,3-benzothiazol-5-yl}pyrimidin-2-yl)piperazin-1-yl]propanoate (Compound No. 33)
MS m/e 597.34 (MH⁺)
1-Ethyl-3-[4-fluoro-7-(3-fluoropyridin-2-yl)-5-{2-[4-(hydroxymethyl)piperidin-1-yl]pyrimidin-5-yl}-1,3-benzothiazol-2-yl]urea (Compound No. 34)
MS m/e 526.27 (MH⁺)
1-Ethyl-3-[4-fluoro-7-(3-fluoropyridin-2-yl)-5-{2-[3-(hydroxymethyl)piperidin-1-yl]pyrimidin-5-yl}-1,3-benzothiazol-2-yl]urea (Compound No. 35)
MS m/e 526.21 (MH⁺)
1-Ethyl-3-[4-fluoro-7-(3-fluoropyridin-2-yl)-5-phenyl-1,3-benzothiazol-2-yl]urea (Compound No. 36)
MS m/e 411.23 (MH⁺)

### Example 2

### Synthesis of 1-ethyl-3-[4-fluoro-7-(pyridin-2-yl)-5-(pyridin-3-yl)-1,3-benzothiazol-2-yl]urea (Compound No. 37)

### Step 1: Synthesis of N-(2-bromo-5-fluorophenyl)-2,2-dimethylpropanamide

Prepared employing procedures as provided in Step 1 of Examples 1

MS m/e 274.48 (MH⁺)

### Step 2: Synthesis of N-[5-fluoro-2-(4,4,5,5-tetramethyl-1,3,2-dioxaborolan-2-yl)phenyl]-2,2-dimethyl-propanamide

Prepared employing procedures as provided in Step 2 of Examples 1

MS m/e 322.73 (MH⁺)

### Step 3: Synthesis of N-[5-fluoro-2-(pyridin-2-yl)phenyl]-2,2-dimethylpropanamide

To a solution of N-[5-fluoro-2-(4,4,5,5-tetramethyl-1,3,2-dioxaborolan-2-yl)phenyl]-2,2-dimethylpropanamide (100.0 g, 300 mmol) in acetonitrile:water (900 mL:100 mL) was added 2-chloropyridine (42.0 g, 373 mmol), Pd(PPh₃)₄ (35.0 g, 0.03 mmol) and potassium carbonate (130.0 g, 934 mmol) in under argon at room temperature (˜25° C.). The reaction mixture was heated at 80 to 85° C. over a period of about 8 hours. The mixture was cooled to room temperature (˜25° C.) and concentrated under reduced pressure. The residue obtained was diluted with water and extracted with ethyl acetate. The organic layer was dried over anhydrous sodium sulfate, filtered and then concentrated under reduced pressure. The crude product obtained was purified through column chromatography using silica gel 230-400 mesh size and eluted with gradient of 5-10% ethyl acetate in hexane provided the title compound (65.0 g).

MS m/e 272.94 (MH⁺)

### Step 4: Synthesis of N-[4-bromo-5-fluoro-2-(pyridin-2-yl)phenyl]-2,2-dimethylpropanamide

To a solution of N-[5-fluoro-2-(pyridin-2-yl)phenyl]-2,2-dimethylpropanamide (64.0 g, 235 mmol) in acetic acid (770 mL) was added bromine (38 mL, 705 mmol) as a solution in 130 mL of acetic acid over a period of about 1 hour. The reaction mixture was stirred at room temperature (˜25° C.) for about 5 hours and then poured over ice, diluted with 1N Na₂S₂O₃/sodium bisulphate (400 mL) and stirred for 30 minute, then filtered and washed with water. The resulting solids were dried under high vacuum to afford the title compound (65 g).

MS m/e 351.16 (MH⁺)

### Step 5: Synthesis of N-[4-bromo-3-fluoro-6-(pyridin-2-yl)-2-nitrophenyl]-2,2-dimethylpropanamide

The solution of N-[4-bromo-5-fluoro-2-(pyridin-2-yl)phenyl]-2,2-dimethylpropanamide (30.0 g, 85.0 mmol) in conc. sulfuric acid was cooled to 5-10° C. and then added nitric acid (fuming, 30 mL) drop wise while maintaining the temperature 5-10° C. After complete addition, reaction mixture was brought up to 15-20° C. and stirred for 30 minute. Again 17 mL of nitric acid was added drop wise, while maintaining the temperature 15-20° C. and stirred for 15-20 minutes. After 20 minutes stirring again 17 ml of nitric acid was added, while maintaining the same temperature and stirred for 15-20 minutes. On the basis of TLC, 8 mL of nitric acid was again added drop wise while maintaining the same temperature and stirred for 15 minutes. The reaction mixture was immediately poured (slowly) in to crushed ice (2.0 lit) and stirred for 30 minutes. The solid separated was filtered, washed with water and dried to get title compound. The filtrate was further extracted with dichloromethane, dried over anhydrous sodium sulfate, filtered and concentrated to recover product from aqueous layer. The combined product were triturated with hexane, filtered and dried under high vacuum to afford title compound (22 g).

MS m/e 396.11 (MH⁺)

### Step 6: Synthesis of 4-bromo-3-fluoro-6-(pyridin-2-yl)-2-nitroaniline

A solution of N-[4-bromo-3-fluoro-6-(pyridin-2-yl)-2-nitrophenyl]-2,2-dimethyl-propanamide (48.0 g, 121.5 mmol) in 70% aq. sulfuric acid (220 mL) was refluxed at 80-90° C. for a period of 30-45 minutes. The reaction mixture was cool to room temperature (−25° C.) and then poured over crushed ice and stirred for about 30 minutes. The resultant solid were filtered, washed with water and then dried under high vacuum to afford title compound (29 g).

MS m/e 312.16 (MH⁺)

### Step 7: Synthesis of 2-(5-bromo-4-fluoro-2-bromo-3-nitrophenyl)-pyridine

To a solution of 4-bromo-3-fluoro-6-(pyridin-2-yl)-2-nitroaniline (1.0 g, 3.2 mmol) in acetonitrile (15 mL) were added cupric bromide (0.46 g, 1.9 mmol) and stirred at 60° C. To this stirred solution, isoamylnitrite (0.7 mL, 4.8 mmol) was added dropwise at the same temperature. After complete addition, reaction mixture was bring to room temperature (˜25° C.) and stirred for about 2 hours. The reaction mixture was concentrated and then diluted with ethylacetate, washed with 0.5 N aq. HCl solution. The organic layer was dried over anhydrous sodium sulfate, filtered and then concentrated under reduced pressure. The crude product obtained was purified through column chromatography using silica gel 100-200 mesh size and eluted with 1% MeOH in dichloromethane to afford title compound (0.65 g).

MS m/e 374.92 (MH⁺)

### Step 8: Synthesis 3-bromo-2-fluoro-5-(pyridin-2-yl)-6-bromoaniline

To a solution of 2-(5-bromo-4-fluoro-2-bromo-3-nitrophenyl)-pyridine (17.0 g, 52.6 mmol) in ethanol and water (165 mL: 165 mL) was added Fe powder (25.0 g, 44.7 mmol), and cone. HCl (5.4 mL). The reaction mixtures were refluxed for about 2 hours and then cool to room temperature (˜25° C.), filtered through celite pad and washed with ethyl acetate. The filtrate was concentrated under reduced pressure and then aqueous layer was extracted with ethyl acetate. The organic layer was dried over anhydrous sodium sulfate, filtered and concentrated to afford title compound (16.0 g).

MS m/e 345.01 (MH⁺)

### Step 9: Synthesis of N-[5-bromo-4-fluoro-7-(pyridin-2-yl)-1,3-benzothiazol-2-yl]acetamide

To a solution of 3-bromo-2-fluoro-5-(pyridin-2-yl)-6-bromoaniline (16.0 g, 46.5 mmol) in acetone (350 mL) was added benzoylisothiocyanate (8.3 g, 51.0 mmol) drop-wise over a period of 20 minutes. The reaction mixture was stirred at room temperature (˜25° C.) for about 10 hours. The solvent was removed under reduced pressure and the residue obtained was triturated with diethyl ether. The solid separated was filtered, dried under high vacuum to afford title compound (14.0 g).

MS m/e 428.10 (MH)

### Step 10: Synthesis of 5-bromo-4-fluoro-7-(pyridin-2-yl)-1,3-benzothiazol-2-amine

To a solution of N-[5-bromo-4-fluoro-7-(pyridin-2-yl)-1,3-benzothiazol-2-yl]acetamide (0.065 g, 0.152 mmol) in methanol (1 mL) was added 2 N aq. NaOH solution (1 mL). The reaction mixture was refluxed at 100-110° C. for about 18 hours. The solvent was removed under reduced pressure and then poured in to water. The solid precipitate was filtered, washed with water and then dried under high vacuum to afford title compound (0.05 g).

MS m/e 324.07 (MH⁺)

### Step 11: Synthesis of S-methyl[5-bromo-4-fluoro-7-(pyridin-2-yl)-1,3-benzothiazol-2-yl]carbamothioate

To a solution of 5-bromo-4-fluoro-7-(pyridin-2-yl)-1,3-benzothiazol-2-amine (0.120 g, 0.366 mmol) in pyridine (4 mL) was added methylchlorothiformate (0.082 g, 0.73 mmol) drop-wise under argon and then heated at 50-55° C. for about 12 hours. The reaction mixture was cooled to room temperature (˜25° C.) and slowly poured in to ice. The solid precipitate was filtered, washed with water and then dried under high vacuum to afford title compound (0.125 g), which is taken as such for next step.

MS m/e 397.98 (MH⁺)

### Step 12: Synthesis of 1-[5-bromo-4-fluoro-7-(pyridin-2-yl)-1,3-benzothiazol-2-yl]-3-ethylurea

To a solution of S-methyl[5-bromo-4-fluoro-7-(pyridin-2-yl)-1,3-benzothiazol-2-yl]carbamothioate (0.125 g, 0.342 mmol) in ethanol (4 mL) was added ethylamine (1.5 mL, 2.0 mL tetrahydrofuran solution) and then refluxed for about 18 hours. The reaction mixture concentrated under reduced pressure. The residue obtained was triturated with water, filtered and dried under high vacuum. The solid residue was again triturated with diethyl ether, filtered and dried under high vacuum to afford title compound (0.100 g).

MS m/e 395.07 (MH⁺)

### Step 13: Synthesis of 1-ethyl-3-[4-fluoro-7-(pyridin-2-yl)-5-(pyridin-3-yl)-1,3-benzothiazol-2-yl]urea

Prepared employing procedures as provided in Step 13 of Examples 1.

¹H NMR (400 MHz, DMSO-d₆) δ: 10.94 (bs, 1H), 8.93 (bs, 1H), 8.80 (bs, 1H), 8.65 (d, 1H, J=4.6 Hz), 8.42 (d, 1H, 7.7 Hz), 8.15 (d, 2H, J=6.2 Hz) 7.97 (bs, 1H), 7.57 (t, 3H, J=4.0 Hz), 7.44 (d, 1H, J=3.8 Hz), 6.73 (bs, 1H), 3.20 (q, 2H), 1.10 (t, 3H).

MS m/e 394.22 (MH⁺).

Following compounds are prepared by following same route of synthesis of Example 2 described above
1-Ethyl-3-[4-fluoro-5-(6-methylpyridin-3-yl)-7-(pyridin-2-yl)-1,3-benzothiazol-2-yl]urea (Compound No. 38)
MS m/e 408.26 (MH⁺)
1-Ethyl-3-[4-fluoro-5-(6-methoxypyridin-3-yl)-7-(pyridin-2-yl)-1,3-benzothiazol-2-yl]urea (Compound No. 39)
MS m/e 424.20 (MH⁺)

### 1-Ethyl-3-[4-fluoro-7-(pyridin-2-yl)-5-(pyrimidin-5-yl)-1,3-benzothiazol-2-yl]urea (Compound No. 40)

MS m/e 395.25 (MH⁺)
1-ethyl-3-[4-fluoro-5-(1-methyl-1H-pyrazol-4-yl)-7-(pyridin-2-yl)-1,3-benzothiazol-2-yl]urea (Compound No. 41)
MS m/e 397.25 (MH⁺)
Methyl 4-{2-[(ethylcarbamoyl)amino]-4-fluoro-7-(pyridin-2-yl)-1,3-benzothiazol-5-yl}benzoate (Compound No. 42)
MS m/e 451.28 (MH⁺)
Methyl 3-{2-[(ethylcarbamoyl)amino]-4-fluoro-7-(pyridin-2-yl)-1,3-benzothiazol-5-yl}benzoate (Compound No. 43)
MS m/e 451.22 (MH⁺)
Ethyl (3-{2-[(ethylcarbamoyl)amino]-4-fluoro-7-(pyridin-2-yl)-1,3-benzothiazol-5-yl}phenoxy)acetate (Compound No. 44)
MS m/e 495.26 (MH⁺)
Ethyl (4-{2-[(ethylcarbamoyl)amino]-4-fluoro-7-(pyridin-2-yl)-1,3-benzothiazol-5-yl}phenoxy)acetate (Compound No. 45)
MS m/e 495.25 (MH⁺)
Methyl 4-{2-[(ethylcarbamoyl)amino]-4-fluoro-7-(pyridin-2-yl)-1,3-benzothiazol-5-yl}-2-methoxybenzoate (Compound No. 46)
MS m/e 481.28 (MH⁺)
Ethyl 1-[(4-{2-[(ethylcarbamoyl)amino]-4-fluoro-7-(pyridin-2-yl)-1,3-benzothiazol-5-yl}phenyl)carbonyl]piperidine-3-carboxylate (Compound No. 47)
MS m/e 576.33 (MH⁺)
Methyl 5-{2-[(ethylcarbamoyl)amino]-4-fluoro-7-(pyridin-2-yl)-1,3-benzothiazol-5-yl}pyridine-3-carboxylate (Compound No. 48)
MS m/e 452.25 (MH⁺)
Methyl (4-{2-[(ethylcarbamoyl)amino]-4-fluoro-7-(pyridin-2-yl)-1,3-benzothiazol-5-yl}-1H-pyrazol-1-yl)acetate (Compound No. 49)
MS m/e 455.21 (MH⁺)
Methyl 4-{2-[(ethylcarbamoyl)amino]-4-fluoro-7-(pyridin-2-yl)-1,3-benzothiazol-5-yl}thiophene-2-carboxylate (Compound No. 50)
MS m/e 457.17 (MH⁺)
1-Ethyl-3-[4-fluoro-5-(6-{[2-(morpholin-4-yl)ethyl]amino}pyridin-3-yl)-7-(pyridin-2-yl)-1,3-benzothiazol-2-yl]urea (Compound No. 51)
MS m/e 522.41 (MH⁺)
1-Ethyl-3-[4-fluoro-5-{3-[(4-methylpiperazin-1-yl)carbonyl]phenyl}-7-(pyridin-2-yl)-1,3-benzothiazol-2-yl]urea (Compound No. 52)
MS m/e 519.31 (MH⁺)
1-Ethyl-3-{4-fluoro-5-[6-(4-methylpiperazin-1-yl)pyridin-3-yl]-7-(pyridin-2-yl)-1,3-benzothiazol-2-yl}urea (Compound No. 53)
MS m/e 449.29 (MH⁺)
1-Ethyl-3-{4-fluoro-5-[2-(4-methylpiperazin-1-yl)pyridin-4-yl]-7-(pyridin-2-yl)-1,3-benzothiazol-2-yl}urea (Compound No. 54)
MS m/e 492.29 (MH⁺)
1-Ethyl-3-[4-fluoro-5-{4-[(4-methylpiperazin-1-yl)carbonyl]phenyl}-7-(pyridin-2-yl)-1,3-benzothiazol-2-yl]urea (Compound No. 55)
MS m/e 519.24 (MH⁺)
1-Ethyl-3-{4-fluoro-5-[2-(piperazin-1-yl)pyridin-3-yl]-7-(pyridin-2-yl)-1,3-benzothiazol-2-yl}urea (Compound No. 56)
MS m/e 478.24 (MH⁺)
1-[5-{6-[3-(Dimethylamino)propoxy]pyridin-3-yl}-4-fluoro-7-(pyridin-2-yl)-1,3-benzothiazol-2-yl]-3-ethylurea (Compound No. 57)
MS m/e 495.32 (MH⁺)
1-Ethyl-3-[4-fluoro-5-{2-[3-(hydroxymethyl)piperidin-1-yl]pyrimidin-5-yl}-7-(pyridin-2-yl)-1,3-benzothiazol-2-yl]urea (Compound No. 58)
MS m/e 508.30 (MH⁺)
1-Ethyl-3-{4-fluoro-5-[6-(morpholin-4-yl)pyridin-3-yl]-7-(pyridin-2-yl)-1,3-benzothiazol-2-yl}urea (Compound No. 59)
MS m/e 479.29 (MH⁺)
tert-Butyl 4-{2-[(ethylcarbamoyl)amino]-4-fluoro-7-(pyridin-2-yl)-1,3-benzothiazol-5-yl}-3,6-dihydropyridine-1(2H)-carboxylate (Compound No. 60)
MS m/e 498.42 (MH⁺)
Ethyl N-(5-{2-[(ethylcarbamoyl)amino]-4-fluoro-7-(pyridin-2-yl)-1,3-benzothiazol-5-yl}pyrimidin-2-yl)-N-methylglycinate (Compound No. 61)
MS m/e 510.28 (MH⁺)
1-{5-[2-(4-Aminopiperidin-1-yl)pyrimidin-5-yl]-4-fluoro-7-(pyridin-2-yl)-1,3-benzothiazol-2-yl}-3-ethylurea hydrochloride salt (Compound No. 62)
MS m/e 493.25 (MH⁺)
1-Ethyl-3-[4-fluoro-5-{2-[(piperidin-4-ylmethyl)amino]pyrimidin-5-yl}-7-(pyridin-2-yl)-1,3-benzothiazol-2-yl]urea hydrochloride salt (Compound No. 63)
MS m/e 507.32 (MH⁺)
Ethyl N-(5-{2-[(ethylcarbamoyl)amino]-4-fluoro-7-(3-fluoropyridin-2-yl)-1,3-benzothiazol-5-yl}pyrimidin-2-yl)glycinate (Compound No. 64)
MS m/e 514.24 (MH⁺)
Methyl 4-{2-[(ethylcarbamoyl)amino]-4-fluoro-7-(3-fluoropyridin-2-yl)-1,3-benzothiazol-5-yl}-2-methoxybenzoate (Compound No. 65)
MS m/e 499.27 (MH⁺)
Ethyl 1-[(4-{2-[(ethylcarbamoyl)amino]-4-fluoro-7-(3-fluoropyridin-2-yl)-1,3-benzothiazol-5-yl}phenyl)carbonyl]piperidine-3-carboxylate (Compound No. 66)
MS m/e 594.35 (MH⁺)
Ethyl (4-{2-[(ethylcarbamoyl)amino]-4-fluoro-7-(3-fluoropyridin-2-yl)-1,3-benzothiazol-5-yl}phenoxy)acetate (Compound No. 67)
MS m/e 513.24 (MH⁺)
Ethyl (3-{2-[(ethylcarbamoyl)amino]-4-fluoro-7-(3-fluoropyridin-2-yl)-1,3-benzothiazol-5-yl}phenoxy)acetate (Compound No. 68)
MS m/e 513.24 (MH⁺)
Methyl 5-{2-[(ethylcarbamoyl)amino]-4-fluoro-7-(3-fluoropyridin-2-yl)-1,3-benzothiazol-5-yl}pyridine-3-carboxylate (Compound No. 69)
MS m/e 470.19 (MH⁺)
Ethyl N-(5-{2-[(ethylcarbamoyl)amino]-4-fluoro-7-(3-fluoropyridin-2-yl)-1,3-benzothiazol-5-yl}pyrimidin-2-yl)-N-methylglycinate (Compound No. 70)
MS m/e 528.21 (MH⁺)
Diethyl[(5-{2-[(ethylcarbamoyl)amino]-4-fluoro-7-(3-fluoropyridin-2-yl)-1,3-benzothiazol-5-yl}pyrimidin-2-yl)amino]propanedioate (Compound No. 71)
MS m/e 586.23 (MH⁺)
Dimethyl N-(5-{2-[(ethylcarbamoyl)amino]-4-fluoro-7-(3-fluoropyridin-2-yl)-1,3-benzothiazol-5-yl}pyrimidin-2-yl)-L-glutamate (Compound No. 72)
MS m/e 586.3 (MH⁺)
1-Ethyl-3-[4-fluoro-7-(3-fluoropyridin-2-yl)-5-{2-[(piperidin-4-ylmethyl)amino]pyrimidin-5-yl}-1,3-benzothiazol-2-yl]urea hydrochloride salt (Compound No. 73)
MS m/e 525.26 (MH⁺)
1-{5-[2-(3-Aminopyrrolidin-1-yl)pyrimidin-5-yl]-4-fluoro-7-(3-fluoropyridin-2-yl)-1,3-benzothiazol-2-yl}-3-ethylurea hydrochloride salt (Compound No. 74)
MS m/e 497.27 (MH⁺)
1-Ethyl-3-{4-fluoro-7-(3-fluoropyridin-2-yl)-5-[2-(piperazin-1-yl)pyrimidin-5-yl]-1,3-benzothiazol-2-yl}urea hydrochloride salt (Compound No. 75)
MS m/e 497.2 (MH⁺)
1-Ethyl-3-{4-fluoro-7-(3-fluoropyridin-2-yl)-5-[2-(piperidin-4-ylamino)pyrimidin-5-yl]-1,3-benzothiazol-2-yl}urea hydrochloride salt (Compound No. 76)
MS m/e 511.23 (MH⁺)
1-{5-[2-(4-Aminopiperidin-1-yl)pyrimidin-5-yl]-4-fluoro-7-(3-fluoropyridin-2-yl)-1,3-benzothiazol-2-yl}-3-ethylurea hydrochloride salt (Compound No. 77)
MS m/e 511.23 (MH⁺)
1-[5-{2-[2-(1,2-Dihydroxy-ethyl)-4,5-dihydroxy-tetrahydro-furan-3-yloxy]-pyrimidin-5-yl}-4-fluoro-7-(3-fluoro-pyridin-2-yl)-benzothiazol-2-yl]-3-ethyl-urea (Compound No. 78)
MS m/e 591.14 (MH⁺)
1-(5-{2-[5-(1,2-Dihydroxy-ethyl)-2,2-dimethyl-tetrahydro-furo[2,3-d][1,3]dioxol-6-yloxy]-pyrimidine-5-yl}-4-fluoro-7-pyridin-2-yl-benzpthiazol-2-yl)-3-ethyl-urea (Compound No. 79)
MS m/e 612.98 (MH⁺)
1-{5-[2-(2,3-Dihydroxypropoxy)pyrimidin-5-yl]-4-fluoro-7-(3-fluoropyridin-2-yl)-1,3-benzothiazol-2-yl}-3-ethylurea (Compound No. 80)
MS m/e 503.12 (MH⁺)
1-Ethyl-3-[4-fluoro-7-(3-fluoropyridin-2-yl)-5-{1-[2-(morpholin-4-yl)ethyl]-1H-pyrazol-4-yl}-1,3-benzothiazol-2-yl]urea hydrochloride salt (Compound No. 81)
MS m/e 514.29 (MH⁺)
1-Ethyl-3-{4-fluoro-5-[6-(piperazin-1-yl)pyridin-3-yl]-7-(pyridin-2-yl)-1,3-benzothiazol-2-yl}urea (Compound No. 82)
MS m/e 478.30 (MH⁺)
1-Ethyl-3-[4-fluoro-7-(3-fluoropyridin-2-yl)-5-{4-[(4-methylpiperazin-1-yl)carbonyl]phenyl}-1,3-benzothiazol-2-yl]urea (Compound No. 83)
MS m/e 537.15 (MH⁺)

### Example 3

### Synthesis of 1-ethyl-3-[4-fluoro-5-(1H-pyrazol-4-yl)-7-(pyridin-2-yl)-1,3-benzothiazol-2-yl]urea Hydrochloride salt (Compound No. 84)

### Step 1: Synthesis of tert-butyl 4-{2-[(ethylcarbamoyl)amino]-4-fluoro-7-(pyridin-2-yl)-1,3-benzothiazol-5-yl}-1H-pyrazole-1-carboxylate

The title compound was prepared by reacting 1-[5-bromo-4-fluoro-7-(pyridin-2-yl)-1,3-benzothiazol-2-yl]-3-ethylurea (0.050 g, 0.126 mmol) and tert-butyl 4-(4,4,5,5-tetramethyl-1,3,2-dioxaborolan-2-yl)-1H-pyrazole-1-carboxylate (0.055 g, 0.19 mmol) in the presence of potassium carbonate and Pd(dppf)Cl₂ dichloromethane complex (1:1) using the same procedure as used in step 13 of example 1. Yield: 0.025 g.

MS m/e 483.24 (MH⁺).

### Step 2: Synthesis of 1-ethyl-3-[4-fluoro-5-(1H-pyrazol-4-yl)-7-(pyridin-2-yl)-1,3-benzothiazol-2-yl]urea Hydrochloride salt

To a solution of 1-ethyl-3-[4-fluoro-5-(1H-pyrazol-4-yl)-7-(pyridin-2-yl)-1,3-benzothiazol-2-yl]urea 0.02 g, 0.041 mmol) in ethanol was added ethanolic HCl (2.5 mL, in excess) at about 0° C. and then stirred at room temperature (˜25° C.) for about 4 hours. The reaction mixture was concentrated under reduced pressure and triturated with ether, solid separated was filtered and dried under high vacuum to afford title compound. Yield: 0.010 mg.

MS m/e 383.24 (MH⁺).

¹H NMR (400 MHz, DMSO-d₆) δ: 10.9 (bs, 1H), 8.79 (d, 1H, J=4.2 Hz), 8.45 (d, 1H, J=8.28 Hz), 8.34-8.28 (m, 3H), 8.00 (t, 1H, J=8.0 Hz), 7.43 (t, 1H, J=4.9 Hz), 6.82 (bs, 1H), 3.20 (q, 2H), 1.05 (t, 3H).

Following compounds are prepared by following same route of synthesis as above
1-Ethyl-3-[4-fluoro-7-(pyridin-2-yl)-5-(1,2,3,6-tetrahydropyridin-4-yl)-1,3-benzothiazol-2-yl]urea Hydrochloride salt (Compound No. 85)
MS m/e 398.27 (MH⁺)
1-Ethyl-3-{4-fluoro-5-[2-(piperazin-1-yl)pyrimidin-5-yl]-7-(pyridin-2-yl)-1,3-benzothiazol-2-yl}urea hydrochloride salt (Compound No. 86)
MS m/e 479.25 (MH⁺)
1-{5-[2-(3-Aminopyrrolidin-1-yl)pyrimidin-5-yl]-4-fluoro-7-(pyridin-2-yl)-1,3-benzothiazol-2-yl}-3-ethylurea hydrochloride salt (Compound No. 87)
MS m/e 479.28 (MH⁺)
1-Ethyl-3-{4-fluoro-5-[2-(piperidin-4-ylamino)pyrimidin-5-yl]-7-(pyridin-2-yl)-1,3-benzothiazol-2-yl}urea hydrochloride salt (Compound No. 88)
MS m/e 493.33 (MH⁺)
Methyl 5-{2-[(ethylcarbamoyl)amino]-4-fluoro-7-(pyridin-2-yl)-1,3-benzothiazol-5-yl}pyridine-2-carboxylate (Compound No. 89)
MS m/e 452.23 (MH⁺)
Methyl (4-{2-[(ethylcarbamoyl)amino]-4-fluoro-7-(pyridin-2-yl)-1,3-benzothiazol-5-yl}phenyl)acetate (Compound No. 90)
MS m/e 465.5 (MH⁺)
Diethyl N-(5-{2-[(ethylcarbamoyl)amino]-4-fluoro-7-(pyridin-2-yl)-1,3-benzothiazol-5-yl}pyrimidin-2-yl)aspartate (Compound No. 91)
MS m/e 582.28 (MH⁺)
Diethyl N-(5-{2-[(ethylcarbamoyl)amino]-4-fluoro-7-(pyridin-2-yl)-1,3-benzothiazol-5-yl}pyrimidin-2-yl)-L-glutamate (Compound No. 92)
MS m/e 596.30 (MH⁺)

### Example 4

### Synthesis of 4-{2-[(ethylcarbamoyl)amino]-4-fluoro-7-(pyridin-2-yl)-1,3-benzothiazol-5-yl}benzoic acid Lithium salt (Compound No. 93)

### Step 1: Synthesis of 4-{2-[(ethylcarbamoyl)amino]-4-fluoro-7-(pyridin-2-yl)-1,3-benzothiazol-5-yl}benzoic acid

To a solution of methyl 4-{2-[(ethylcarbamoyl)amino]-4-fluoro-7-(pyridin-2-yl)-1,3-benzothiazol-5-yl}benzoate (0.025 g, 0.059 mmol) in methanol was added 2N NaOH solution and then stirred at room temperature (˜25° C.) for about 6 hours. The reaction mixture was concentrated under reduced pressure. The residue obtained was taken in water, acidified with aq. HCl. The solid separated was filtered, washed with water and dried under high vacuum to get title compound (0.020 g).

MS m/e 437.24 (MH⁺).

### Step 2: Synthesis of 4-{2-[(ethylcarbamoyl)amino]-4-fluoro-7-(pyridin-2-yl)-1,3-benzothiazol-5-yl}benzoic acid Lithium salt

To solution of 4-{2-[(ethylcarbamoyl)amino]-4-fluoro-7-(pyridin-2-yl)-1,3-benzothiazol-5-yl}benzoic acid (0.018 g, 0.041 mmol) in tetrahydrofuran were added lithium hydroxide and then stirred at room temperature (˜25° C.) for about 18 hours. the reaction mixture was concentrated and triturated with ether, solid separated was filtered and dried to get title compound (0.015 g).

MS m/e 437.24 (MH⁺).

¹H NMR (400 MHz, DMSO-d₆) δ: 11.0 (bs, 1H), 8.81 (d, 111, J=4.6 Hz), 8.40 (d, 1H, J=8.28 Hz), 8.13-8.08 (m, 3H), 7.97 (t, 1H, J=8 Hz), 7.87 (d, 2H, J=7.68 Hz), 7.44 (t, 1H, J=4.9 Hz), 6.79 (bs, 1H), 3.21 (q, 2H), 1.11 (t, 3H).

Following compounds are prepared by following same route of synthesis as above
3-{2-[(Ethylcarbamoyl)amino]-4-fluoro-7-(pyridin-2-yl)-1,3-benzothiazol-5-yl}benzoic acid Lithium salt (Compound No. 94)
MS m/e 437.24 (MH⁺)
(3-{2-[(Ethylcarbamoyl)amino]-4-fluoro-7-(pyridin-2-yl)-1,3-benzothiazol-5-yl}phenoxy)acetic acid Lithium salt (Compound No. 95)
MS m/e 467.23 (MH⁺)
(4-{2-[(Ethylcarbamoyl)amino]-4-fluoro-7-(pyridin-2-yl)-1,3-benzothiazol-5-yl}phenoxy)acetic acid Lithium salt (Compound No. 96)
MS m/e 467.23 (MH⁺)
4-{2-[(Ethylcarbamoyl)amino]-4-fluoro-7-(pyridin-2-yl)-1,3-benzothiazol-5-yl}-2-methoxybenzoic acid (Compound No. 97)
MS m/e 467.23 (MH⁺)
1-[(4-{2-[(Ethylcarbamoyl)amino]-4-fluoro-7-(pyridin-2-yl)-1,3-benzothiazol-5-yl}phenyl)carbonyl]piperidine-3-carboxylic acid Lithium salt (Compound No. 98)
MS m/e 548.23 (MH⁺)
5-{2-[(Ethylcarbamoyl)amino]-4-fluoro-7-(pyridin-2-yl)-1,3-benzothiazol-5-yl}pyridine-3-carboxylic acid Lithium salt (Compound No. 99)
MS m/e 438.19 (MH⁺)
(4-{2-[(Ethylcarbamoyl)amino]-4-fluoro-7-(pyridin-2-yl)-1,3-benzothiazol-5-yl}-1H-pyrazol-1-yl)acetic acid lithium salt (Compound No. 100)
MS m/e 441.29 (MH⁺)
4-{2-[(ethylcarbamoyl)amino]-4-fluoro-7-(pyridin-2-yl)-1,3-benzothiazol-5-yl}thiophene-2-carboxylic acid Lithium salt (Compound No. 101)
MS m/e 443.18 (MH⁺)
5-{2-[(Ethylcarbamoyl)amino]-4-fluoro-7-(pyridin-2-yl)-1,3-benzothiazol-5-yl}pyridine-2-carboxylic acid Lithium salt (Compound No. 102)
MS m/e 438.25 (MH⁺)
(4-{2-[(Ethylcarbamoyl)amino]-4-fluoro-7-(pyridin-2-yl)-1,3-benzothiazol-5-yl}phenyl)acetic acid Lithium salt (Compound No. 103)
MS m/e 451.22 (MH⁺)
1-(5-{2-[(Ethylcarbamoyl)amino]-4-fluoro-7-(pyridin-2-yl)-1,3-benzothiazol-5-yl}pyrimidin-2-yl)piperidine-4-carboxylic acid Lithium salt (Compound No. 104)
MS m/e 522.28 (MH⁺)
5-{2-[(1-Carboxy-2-methoxy-2-oxoethyl)amino]pyrimidin-5-yl}-2-[(ethylcarbamoyl)amino]-4-fluoro-7-(pyridin-2-yl)-1,3-benzothiazole Lithium salt (Compound No. 105)
MS m/e 526.27 (MH⁺)
N-(5-{2-[(Ethylcarbamoyl)amino]-4-fluoro-7-(pyridin-2-yl)-1,3-benzothiazol-5-yl}pyrimidin-2-yl)aspartic acid Lithium salt (Compound No. 106)
MS m/e 526.27 (MH⁺)
N-(5-{2-[(Ethylcarbamoyl)amino]-4-fluoro-7-(pyridin-2-yl)-1,3-benzothiazol-5-yl}pyrimidin-2-yl)glycine Lithium salt (Compound No. 107)
MS m/e 468.18 (MH⁺)
4-[(3-{2-[(Ethylcarbamoyl)amino]-4-fluoro-7-(pyridin-2-yl)-1,3-benzothiazol-5-yl}phenyl)amino]-4-oxobutanoic acid Lithium salt (Compound No. 108)
MS m/e 508.22 (MH⁺)
4-{2-[(Ethylcarbamoyl)amino]-4-fluoro-7-(3-fluoropyridin-2-yl)-1,3-benzothiazol-5-yl}-2-methoxybenzoic acid (Compound No. 109)
MS m/e 485.23 (MH⁺)
1-[(4-{2-[(Ethylcarbamoyl)amino]-4-fluoro-7-(3-fluoropyridin-2-yl)-1,3-benzothiazol-5-yl}phenyl)carbonyl]piperidine-3-carboxylic acid Lithium salt (Compound No. 110)
MS m/e 566.21 (MH⁺)
(4-{2-[(Ethylcarbamoyl)amino]-4-fluoro-7-(3-fluoropyridin-2-yl)-1,3-benzothiazol-5-yl}phenoxy)acetic acid Lithium salt (Compound No. 111)
MS m/e 485.17 (MH⁺)
5-{2-[(Ethylcarbamoyl)amino]-4-fluoro-7-(3-fluoropyridin-2-yl)-1,3-benzothiazol-5-yl}pyridine-3-carboxylic acid Lithium salt (Compound No. 112)
MS m/e 456.18 (MH⁺)
N-(5-{2-[(Ethylcarbamoyl)amino]-4-fluoro-7-(3-fluoropyridin-2-yl)-1,3-benzothiazol-5-yl}pyrimidin-2-yl)glycine Lithium salt (Compound No. 113)
MS m/e 486.17 (MH⁺)
N-(5-{2-[(Ethylcarbamoyl)amino]-4-fluoro-7-(3-fluoropyridin-2-yl)-1,3-benzothiazol-5-yl}pyrimidin-2-yl)-N-methylglycine Lithium salt (Compound No. 114)
MS m/e 500.26 (MH⁺)
(3-{2-[(Ethylcarbamoyl)amino]-4-fluoro-7-(3-fluoropyridin-2-yl)-1,3-benzothiazol-5-yl}phenoxy)acetic acid Lithium salt (Compound No. 115)
MS m/e 485.17 (MH⁺)
N-(5-{2-[(Ethylcarbamoyl)amino]-4-fluoro-7-(pyridin-2-yl)-1,3-benzothiazol-5-yl}pyrimidin-2-yl)-N-methylglycine Lithium salt (Compound No. 116)
MS m/e 482.21 (MH⁺)

### Example 5

### Synthesis of methyl[4-(5-{2-[(ethylcarbamoyl)amino]-4-fluoro-7-(pyridin-2-yl)-1,3-benzothiazol-5-yl}pyridin-2-yl)piperazin-1-yl]acetate hydrochloride salt (Compound No. 117)

### Step 1: Synthesis of methyl[4-(5-{2-[(ethylcarbamoyl)amino]-4-fluoro-7-(pyridin-2-yl)-1,3-benzothiazol-5-yl}pyridin-2-yl)piperazin-1-yl]acetate

To a solution of 1-ethyl-3-{4-fluoro-5-[6-(piperazin-1-yl)pyridi7n-3-yl]-7-(pyridin-2-yl)-1,3-benzothiazol-2-yl}urea (0.025 g, 0.052 mmol) in tetrahydrofuran (2 mL) were added methylbromo acetate (0.011 g, 0.068 mmol), and potassium carbonate (0.011 g, 0.078 mmol) at about 0° C. The mixture was stirred at room temperature (˜25° C.) for about 12 hours. The solvent was remove under reduced pressure and then purified through column chromatography using silica gel 100-200 mesh size and eluted with 2% methanol in dichloromethane to afford the title compound (0.17 g).

MS m/e 550.31 (MH⁺).

### Step 2: Synthesis of methyl[4-(5-{2-[(ethylcarbamoyl)amino]-4-fluoro-7-(pyridin-2-yl)-1,3-benzothiazol-5-yl}pyridin-2-yl)piperazin-1-yl]acetate hydrochloride salt

To a solution of methyl[4-(5-{2-[(ethylcarbamoyl)amino]-4-fluoro-7-(pyridin-2-yl)-1,3-benzothiazol-5-yl}pyridin-2-yl)piperazin-1-yl]acetate (0.012 g, 0.021 mmol) in ethanol (2 mL) was added ethanolic.HCl (16% w/v, 0.005 mL, 0.022 mmol) at about 0° C. The mixture was stirred at room temperature (˜25° C.) for about 4 hours. The solvent was removed under reduced pressure and then triturated with ether and then filtered, dried under high vacuum to afford the title compound (0.010 g).

¹H NMR (400 MHz, DMSO-d₆) δ: 10.97 (bs, 1H), 8.81 (d, 1H, J=4.2 Hz), 8.55 (s, 1H), 8.39 (d, 1H, J=8.0 Hz), 8.09-7.97 (m, 3H), 7.44 (t, 1H, J=5.4 Hz), 7.16 (d, 1H, J=8.0 Hz), 6.96 (bs, 1H), 4.44 (s, 3H), 3.92 (s, 2H), 3.63-3.45 (m, 6H), 3.23 (q, 2H), 3.14-3.11 (m, 2H), 1.11 (t, 3H).

MS m/e 550.31 (MH⁺).

### Example 6

### Synthesis of [4-(5-{2-[(ethylcarbamoyl)amino]-4-fluoro-7-(pyridin-2-yl)-1,3-benzothiazol-5-yl}pyrimidin-2-yl)piperazin-1-yl]acetic acid Lithium salt (Compound No. 118)

### Step 1: Synthesis of methyl[4-(5-{2-[(ethylcarbamoyl)amino]-4-fluoro-7-(pyridin-2-yl)-1,3-benzothiazol-5-yl}pyrimidin-2-yl)piperazin-1-yl]acetate

To a solution of 1-ethyl-3-{4-fluoro-5-[2-(piperazin-1-yl)pyrimidin-5-yl]-7-(pyridin-2-yl)-1,3-benzothiazol-2-yl}urea (0.025 g, 0.0523 mmol) in tetrahydrofuran (2 mL) were added methylbromo acetate (0.012 g, 0.068 mmol), and potassium carbonate (0.012 g, 0.078 mmol) at about 0° C. The mixture was stirred at room temperature (−25° C.) for about 12 hours. The solvent was remove under reduced pressure and then purified through column chromatography using silica gel 100-200 mesh size and eluted with 2% methanol in dichloromethane to afford the title compound (0.15 g).

MS m/e 551.33 (MH⁺).

### Step 2: Synthesis of [4-(5-{2-[(ethylcarbamoyl)amino]-4-fluoro-7-(pyridin-2-yl)-1,3-benzothiazol-5-yl}pyrimidin-2-yl)piperazin-1-yl]acetic acid

To a solution of compound obtained from step 1 (0.010 g, 0.018 mmol) in methanol was added 2N NaOH solution and then stirred at room temperature (˜25° C.) for about 6 hours. The reaction mixture was concentrated under reduced pressure. The residue obtained was taken in water, acidified with aq. HCl. The solid separated was filtered, washed with water and dried under high vacuum to get title compound (0.008 g).

MS m/e 537.27 (MH⁺).

### Step 3: Synthesis of [4-(5-{2-[(ethylcarbamoyl)amino]-4-fluoro-7-(pyridin-2-yl)-1,3-benzothiazol-5-yl}pyrimidin-2-yl)piperazin-1-yl]acetic acid Lithium salt

To solution of compound obtained from step 2 (0.008 g, 0.014 mmol) in tetrahydrofuran, was added lithium hydroxide and then stirred at room temperature (˜25° C.) for about 18 hours. The reaction mixture was concentrated and triturated with ether, solid separated was filtered and dried to get title compound (0.008 g).

¹H NMR (400 MHz, DMSO-d₆) δ: 8.76-8.70 (m, 3H), 8.26 (s, 1H), 7.94 (t, 2H, J=8.0 Hz), 7.39 (s, 1H), 4.04 (bs, 4H), 3.34-3.14 (m, 8H), 1.06 (t, 3H).

MS m/e 537.27 (MH⁺).

Following compounds are prepared by following the similar route of synthesis as above
1-Ethyl-3-[4-fluoro-5-{2-[4-(2-hydroxyethyl)piperazin-1-yl]pyrimidin-5-yl}-7-(pyridin-2-yl)-1,3-benzothiazol-2-yl]urea hydrochloride salt (Compound No. 119)
MS m/e 523.34 (MH⁺)
3-[4-(5-{2-[(Ethylcarbamoyl)amino]-4-fluoro-7-(pyridin-2-yl)-1,3-benzothiazol-5-yl}pyrimidin-2-yl)piperazin-1-yl]propanoic acid (Compound No. 120)
MS m/e 565.30 (MH⁺)
3-[4-(5-{2-[(Ethylcarbamoyl)amino]-4-fluoro-7-(pyridin-2-yl)-1,3-benzothiazol-5-yl}pyrimidin-2-yl)piperazin-1-yl]propanoic acid Lithium salt (Compound No. 121)
MS m/e 551.23 (MH⁺)
Ethyl 3-[4-(5-{2-[(ethylcarbamoyl)amino]-4-fluoro-7-(pyridin-2-yl)-1,3-benzothiazol-5-yl}pyridin-2-yl)piperazin-1-yl]propanoate hydrochloride salt (Compound No. 122)
MS m/e 578.31 (MH⁺)

### Example 7

### Synthesis of 1-ethyl-3-{4-fluoro-5-[4-(morpholin-4-ylmethyl)phenyl]-7-(pyridin-2-yl)-1,3-benzothiazol-2-yl}urea hydrochloride salt (Compound No. 124)

### Step 1: Synthesis of [4-fluoro-5-(4-formyl-phenyl)-7-pyridine-2-yl-benzothiazol-2-yl]-urea

The title compound was prepared by reacting 1-[5-bromo-4-fluoro-7-(pyridin-2-yl)-1,3-benzothiazol-2-yl]-3-ethylurea and 4-carboxaldehyde-phenyl boronic acid in the presence of potassium carbonate and Pd(dppf)Cl₂ dichloromethane complex (1:1) using the same procedure as used for compound 13 (scheme 1). Yield: 0.3 g.

MS m/e 421.19 (MH⁺).

### Step 2: Synthesis of 1-ethyl-3-{4-fluoro-5-[4-(morpholin-4-ylmethyl)phenyl]-7-(pyridin-2-yl)-1,3-benzothiazol-2-yl}urea

To a solution of [4-fluoro-5-(4-formyl-phenyl)-7-pyridine-2-yl-benzothiazol-2-yl]-urea (0.025 g, 0.059 mmol) in methanol were added morpholine (0.01 mL, 0.119 mmol), acetic acid (0.02 mL, 0.89 mmol) at room temperature (˜25° C.). After about 15 minutes stirring, sodiumcyanoborohydride was added and then stirred for about 12 hours at room temperature (˜25° C.). The solvent was removed under reduced pressure and then purified through column chromatography using silica gel 100-200 mesh size and eluted with 2% methanol in dichloromethane to afford the title compound (0.024 g).

MS m/e 492.29 (MH⁺).

### Step 3: Synthesis of 1-ethyl-3-{4-fluoro-5-[4-(morpholin-4-ylmethyl)phenyl]-7-(pyridin-2-yl)-1,3-benzothiazol-2-yl}urea hydrochloride salt

To a solution of 1-ethyl-3-{4-fluoro-5-[4-(morpholin-4-ylmethyl)phenyl]-7-(pyridin-2-yl)-1,3-benzothiazol-2-yl}urea (0.023 g, 0.046 mmol) in ethanole (2 mL) was added ethanolic.HCl (16% w/v, 0.012 mL, 0.051 mmol) at 0° C. The mixture was stirred at room temperature (˜25° C.) for about 4 hours. The solvent was removed under reduced pressure and then triturated with ether and then filtered, dried under high vacuum to afford the title compound (0.018 g).

¹H NMR (400 MHz, DMSO-d₆) δ: 11.00 (bs, 1H), 8.82 (d, 1H, J=4.4 Hz), 8.38 (d, 1H, J=−8.0 Hz), 8.09 (d, 1H, J=8.0 Hz), 7.98 (t, 1H, J=8.0 Hz), 7.86 (d, 2H, J=8.0 Hz), 7.74 (d, 2H, J=8.0 Hz), 7.46-7.43 (m, 1H), 6.87 (bs, 1H), 4.44 (s, 2H), 4.00-3.97 (m, 2H), 3.79-3.73 (m, 2H), 3.32-3.14 (m, 6H), 1.11 (t, 3H)

MS m/e 492.29 (MH⁺).

### Example 8

### Synthesis of methyl N-[(4-{2-[(ethylcarbamoyl)amino]-4-fluoro-7-(pyridin-2-yl)-1,3-benzothiazol-5-yl}thiophen-2-yl)methyl]-L-serinate hydrochloride salt (Compound No. 130)

### Step 1: Synthesis of 1-ethyl-3-[4-fluoro-5-(5-formylthiophen-3-yl)-7-(pyridin-2-yl)-1,3-benzothiazol-2-yl]urea

The title compound was prepared by reacting 1-[5-bromo-4-fluoro-7-(pyridin-2-yl)-1,3-benzothiazol-2-yl]-3-ethylurea (0.4 g, 1.0 mmol) and 5-formylthiophene-3-boronic acid (0.19 g, 1.2 mmol) in the presence of potassium carbonate (0.7 g, 5.0 mmol) and Pd(dppf)Cl₂ dichloromethane complex (1:1) (0.082 g, 0.1 mmol) using the same procedure as used for compound 13 (scheme 1). Yield: 0.32 g.

¹H NMR (400 MHz, DMSO-d₆) δ: 8.49 (bs, 1H), 7.65 (S, 1H), 7.43-7.36 (m, 3H), 7.30 (d, 1H, J=7.0 Hz), 6.50 (s, 1H), 6.43 (d, 1H, J=3.0 Hz), 5.84 (bs, 1H), 3.59-3.52 (m, 2H), 1.14 (d, 3H).

MS m/e 427.21 (MH⁺).

### Step 2: Synthesis of methyl N-[(4-{2-[(ethylcarbamoyl)amino]-4-fluoro-7-(pyridin-2-yl)-1,3-benzothiazol-5-yl}thiophen-2-yl)methyl]L-serinate

To a solution of 1-ethyl-3-[4-fluoro-5-(5-formylthiophen-3-yl)-7-(pyridin-2-yl)-1,3-benzothiazol-2-yl]urea (0.030 g, 0.070 mmol) in methanol were added L-Serine methyl ester hydrochloride (0.021 g, 0.140 mmol), acetic acid (0.06 mL, 0.10 mmol) at room temperature (˜25° C.). After about 15 minutes stirring, sodiumcyanoborohydride was added and then stirred for about 12 hours at room temperature (˜25° C.). The solvent was removed under reduced pressure and then purified through column chromatography using silica gel 100-200 mesh size and eluted with 2% methanol in dichloromethane to afford the title compound (0.019 g).

MS m/e 530.33 (MH⁺).

### Step 3: Synthesis of methyl N-[(4-{2-[(ethylcarbamoyl)amino]-4-fluoro-7-(pyridin-2-yl)-1,3-benzothiazol-5-yl}thiophen-2-yl)methyl]-serinate hydrochloride salt

To a solution of methyl N-[(4-{2-[(ethylcarbamoyl)amino]-4-fluoro-7-(pyridin-2-yl)-1,3-benzothiazol-5-yl}thiophen-2-yl)methyl]-L-serinate (0.015 g, 0.056 mmol) in ethanol (2 mL) was added ethanolic.HCl (16% w/v, 0.015 mL, 0.068 mmol) at about 0° C. The mixture was stirred at room temperature (˜25° C.) for about 4 hours. The solvent was removed under reduced pressure and then triturated with ether and then filtered, dried under high vacuum to afford the title compound (0.013 g).

¹H NMR (400 MHz, DMSO-d₆) δ: 11.00 (bs, 1H), 9.83 (bs, 1H), 8.79 (d, 1H, J=4.0 Hz), 8.36 (d, 1H, J=8.0 Hz), 8.18 (m, 2H), 7.98 (t, 2H, J=7.0 Hz), 7.90 (s, 1H), 7.45-7.42 (m, 1H), 6.90 (bs, 1H), 4.52 (s, 2H), 4.18 (s, 1H), 3.93-3.89 (m, 31-1), 3.76 (s, 2H), 3.19 (t, 2H), 1.11 (t, 3H).

MS m/e 530.26 (MH⁺)

Following compounds are prepared by following the similar route of synthesis as above
1-Ethyl-3-[4-fluoro-5-{4-[(4-methylpiperazin-1-yl)methyl]phenyl}-7-(pyridin-2-yl)-1,3-benzothiazol-2-yl]urea (Compound No. 123)
MS m/e 505.34 (MH⁺)
1-Ethyl-3-[4-fluoro-5-(4-{[4-(2-hydroxyethyl)piperazin-1-yl]methyl}phenyl)-7-(pyridin-2-yl)-1,3-benzothiazol-2-yl]urea hydrochloride salt (Compound No. 125)
MS m/e 535.32 (MH⁺)
1-Ethyl-3-[4-fluoro-5-{4-[(4-hydroxypiperidin-1-yl)methyl]phenyl}-7-(pyridin-2-yl)-1,3-benzothiazol-2-yl]urea hydrochloride salt (Compound No. 126)
MS m/e 506.34 (MH⁺)
1-Ethyl-3-[4-fluoro-5-(4-{[4-(hydroxymethyl)piperidin-1-yl]methyl}phenyl)-7-(pyridin-2-yl)-1,3-benzothiazol-2-yl]urea hydrochloride salt (Compound No. 127)
MS m/e 520.32 (MH⁺)
1-Ethyl-3-[4-fluoro-5-(5-{[4-(2-hydroxyethyl)piperazin-1-yl]methyl}thiophen-3-yl)-7-(pyridin-2-yl)-1,3-benzothiazol-2-yl]urea hydrochloride salt (Compound No. 128)
MS m/e 541.30 (MH⁺)
Ethyl 1-[(4-{2-[(ethylcarbamoyl)amino]-4-fluoro-7-(pyridin-2-yl)-1,3-benzothiazol-5-yl}thiophen-2-yl)methyl]piperidine-4-carboxylate hydrochloride salt (Compound No. 129)
MS m/e 568.25 (MH⁺)
Ethyl N-[(4-{2-[(ethyl carbamoyl)amino]-4-fluoro-7-(pyridin-2-yl)-1,3-benzothiazol-5-yl}thiophen-2-yl)methyl]-L-alaninate hydrochloride salt (Compound No. 131)
MS m/e 528.29 (MH⁺)
1-Ethyl-3-[4-fluoro-5-{5-[(4-hydroxypiperidin-1-yl)methyl]thiophen-3-yl}-7-(pyridin-2-yl)-1,3-benzothiazol-2-yl]urea hydrochloride salt (Compound No. 132)
MS m/e 512.25 (MH⁺)
1-Ethyl-3-{4-fluoro-5-[5-({[2-(morpholin-4-yl)ethyl]amino}methyl)thiophen-3-yl]-7-(pyridin-2-yl)-1,3-benzothiazol-2-yl}urea hydrochloride salt (Compound No. 133)
MS m/e 541.30 (MH⁺)
1-[5-(5-{[bis(2-Hydroxyethyl)amino]methyl}thiophen-3-yl)-4-fluoro-7-(pyridin-2-yl)-1,3-benzothiazol-2-yl]-3-ethyl urea hydrochloride salt (Compound No. 134)
MS m/e 516.19 (MH⁺)
1-Ethyl-3-{4-fluoro-5-[5-(morpholin-4-ylmethyl)thiophen-3-yl]-7-(pyridin-2-yl)-1,3-benzothiazol-2-yl}urea hydrochloride salt (Compound No. 135)
MS m/e 498.25 (MH⁺)
1-Ethyl-3-[4-fluoro-5-{5-[(4-methylpiperazin-1-yl)methyl]thiophen-3-yl}-7-(pyridin-2-yl)-1,3-benzothiazol-2-yl]urea hydrochloride salt (Compound No. 136)
MS m/e 511.33 (MH⁺)
1-Ethyl-3-[4-fluoro-5-(5-{[4-(2-hydroxyethyl)piperazin-1-yl]methyl}thiophen-2-yl)-7-(pyridin-2-yl)-1,3-benzothiazol-2-yl]urea hydrochloride salt (Compound No. 137)
MS m/e 541.30 (MH⁺)
Ethyl 1-[(5-{2-[(ethylcarbamoyl)amino]-4-fluoro-7-(pyridin-2-yl)-1,3-benzothiazol-5-yl}thiophen-2-yl)methyl]piperidine-4-carboxylate hydrochloride salt (Compound No. 138)
MS m/e 568.25 (MH⁺)

### Assay Methods

### DNA Supercoiling Inhibition Assay (Gyrase Inhibition Assay):

Gyrase supercoiling assays was performed as described by Inspiralis, Norwich, UK (Inspiralis Product No. #G1001). Samples (30 μl) containing 1 unit of DNA gyrase and 0.5 μg of relaxed pBR322 DNA in assay buffer (35 mM Tris-HCl, pH 7.5, 24 mM KCl, 4 mM MgCl₂, 2 mM DTT, 1.8 mM spermidine, 1 mM ATP, 6.5% glycerol and 0.1 mg/ml albumin) was incubated at 37° C. for 30 mM with and without inhibitors. Samples was loaded onto 0.8% agarose gels and run in the absence of ethidium bromide. The gels was stained in ethidium bromide and visualized in Bio-rad gel doc system. The conversion or inhibition of supercoiling DNA was estimated from the bands visible and the IC₅₀ was calculated using Bio-Rad's Quantity one software.

The compounds provided herein showed activity (IC₅₀) between 0.03 μM-2 μM. More specifically, the compounds showed a range of activity between 0.03 μM-0.55 μM.

### DNA Relaxation Inhibition Assay (TopoIV Inhibition Assay):

DNA relaxation assays was performed as described by Inspiralis, Norwich, UK (Inspiralis Product No. #D4001). Samples (30 μl) containing 1 unit of Topoisomerase IV and 0.4 μg of supercoiled pBR322 DNA in assay buffer (40 mM HEPES-KOH, pH 7.6, 100 mM Potassium Glutamate, 10 mM Mg acetate, 10 mM DTT, 2 mM ATP and 50 μg/ml albumin) was incubated at 37° C. for 30 mM with and without inhibitors. Samples was loaded onto 0.8% agarose gels and run in the absence of ethidium bromide. The gels was stained in ethidium bromide and visualized in Bio-rad gel doc system. The conversion or inhibition of supercoiling DNA was estimated from the bands visible and the IC₅₀ was calculated using Bio-Rad's Quantity one software

The compounds provided herein showed activity (IC₅₀) between 0.03 μM-15 μM. More specifically, the compounds showed a range of activity between 0.03 μM-0.55 μM.

### In Vitro Antibacterial Activity (MIC Method)

### Microbroth Dilution Method

### Medium:

1. Cation adjusted Mueller Hinton Broth (MHB-Difco): staphylococci spp., enterococci spp.
2. Cation adjusted Mueller Hinton Broth +5% lysed horse blood for fastidious pathogens: streptococci spp.

### Preparation of Compounds:

1 mg/mL of stock solution of compounds and standard drug were prepared in dimethylsulfoxide/distilled water/solvent and further 2 fold dilutions were done in respective broth in 96 well microtiter plates as per CLSI guidelines. The stock solution was changed according to the need of the experiment.

### Inoculum Preparation:

Saline suspensions was prepared from three to four isolated colonies taken from 18-24 hrs agar plates. The turbidity of the suspension was adjusted to 0.5-1.0 Mc Farland standard (1.5×10⁸ CFU/mL) Cultures was diluted 100 times (respective medium) and 100 μl of diluted culture broth was added in wells already containing 100 μl of broth (positive control) or broth containing compound to get approximately 3−7×10⁵ CFU/ml. Cultures was randomly selected for CFU determination of inoculum suspensions. Micro titer plates was then incubated at 35-37° C. for 16-20 hrs in ambient air.

End Point Determination of MIC:

Concentration of the drug at which there was complete disappearance of growth was considered as MIC.

### REFERENCES

1. Clinical and Laboratory Standards Institute, Methods for Dilution Antimicrobial Susceptibility Tests for Bacteria That Grow Aerobically; Approved Standard-Seventh Edition. M7-A7, Vol. 26. No. 2 (January 2006)
2) Clinical and Laboratory Standards Institute, Performance Standards for Antimicrobial Susceptibility Testing—Sixteenth informational supplement, M100-S17, Vol. 27 No. 1, January 2007

### Results:

a) The compounds described herein exhibited MIC values against Staphylococcus aureus (ATCC29213), S. aureus (ATCC MRSA 43300), S. aureus (MRSA 562), in the range of between about 0.03 μg/mL to about 16 μg/mL, for example between about 0.03 μg/mL to about 2 μg/mL.
b) The compounds described herein exhibited MIC values against S. epidermidis (ATCC 12228) in the range of between about 0.008 μg/mL to about 16 μg/mL, for example between about 0.008 μg/mL to about 1 μg/mL.
c) The compounds described herein exhibited MIC values against S. epidermidis (ATCC MRSE 35984) in the range of between about 0.03 μg/mL to about 16 μg/mL, for example between about 0.03 μg/mL to about 2 μg/mL.
d) The compounds described herein exhibited MIC values against Streptococcus pyogenes (ATCC 19615) in the range of between about 0.06 μg/mL to about 16 μg/mL, for example between about 0.06 μg/mL to about 2 μg/mL.
e) The compounds described herein exhibited MIC values against Streptococcus pyogenes (2534) in the range of between about 0.03 μg/mL to about 16 μg/mL, for example between about 0.03 μg/mL to about 2 μg/mL.
f) The compounds described herein exhibited MIC values against S. viridans (659) in the range of between about 0.03 μg/mL to about 16 μg/mL, for example between about 0.03 μg/mL to about 2 μg/mL.
g) The compounds described herein exhibited MIC values against Streptococcus pneumoniae (ATCC 49619) in the range of between about 0.03 μg/mL to about 4 μg/mL, for example between about 0.03 μg/mL to about 0.5 μg/mL.
h) The compounds described herein exhibited MIC values against E. faecalis (ATCC 29212) in the range of between about 0.03 μg/mL to about 16 μg/mL, for example between about 0.03 μg/mL to about 2 μg/mL.
i) The compounds described herein exhibited MIC values against E. faecium (6A VRE) in the range of between about 0.03 μg/mL to about 16 μg/mL, for example between about 0.03 μg/mL to about 2 μg/mL.

We claim:

1. Compounds having the structure of Formula I 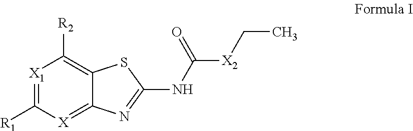
and its pharmaceutically acceptable salts,
wherein
X is N, —C—F or —CCOOR_{f};
X₁ is —CH—, N, —C—F or —CCOOR_{f};
X₂ is —NH— or —O—;
R₁ is cycloalkyl, aryl, heteroaryl or heterocyclyl;
R₂ is hydrogen, alkyl, cycloalkyl, aryl, aralkyl, heterocyclyl substituted alkyl, heteroaryl, heterocyclyl, —NR_{f}R_{q}, —CONR_{f}R_{q}, —COR_{f}, —SO₂R_{f}—COOR_{f}, —CR_{f}═NOR_{f} or —OCONR_{f}R_{q};
wherein R_{f} and R_{q} are independently selected from to hydrogen, alkyl, cycloalkyl, aryl, heteroaryl and heterocyclyl;
and wherein said alkyl, cycloalkyl, aryl, aralkyl, heterocyclyl substituted alkyl, heteroaryl, heterocyclyl groups have 1 to 20 carbon atoms, and with the proviso that
when R₂ is —NR_{f}R_{q}, —CH₂NR_{f}R_{q} or —CONR_{f}R_{q} and R_{f} is heterocyclyl or —CH₂— heterocyclyl then the said heterocyclyl cannot be 1-aza-bicyclo[2.2.2]octane.
when R₂ is —NR_{f}R_{q} wherein R_{f} is heteroaryl and R₁ is phenyl or pyridinyl then it cannot be further substituted with —CH₂-thiazolidinedione or —CH₂-dialkoxy.

2. A compound selected from the group consisting of:
1-Ethyl-3-[4-fluoro-7-(3-fluoropyridin-2-yl)-5-(pyridin-3-yl)-1,3-benzothiazol-2-yl]urea (Compound No. 1),
1-Ethyl-3-[4-fluoro-7-(3-fluoropyridin-2-yl)-5-(6-methyloxypyridin-3-yl)-1,3-benzothiazol-2-yl]urea (Compound No. 2),
1-Ethyl-3-[4-fluoro-7-(3-fluoropyridin-2-yl)-5-(6-methylpyridin-3-yl)-1,3-benzothiazol-2-yl]urea (Compound No. 3),
1-Ethyl-3-[4-fluoro-7-(3-fluoropyridin-2-yl)-5-(pyridin-4-yl)-1,3-benzothiazol-2-yl]urea (Compound No. 4),
1-Ethyl-3-[4-fluoro-7-(3-fluoropyridin-2-yl)-5-(5-fluoropyridin-3-yl)-1,3-benzothiazol-2-yl]urea (Compound No. 5),
1-Ethyl-3-[4-fluoro-7-(3-fluoropyridin-2-yl)-5-(1-methyl-1H-pyrazol-4-yl)-1,3-benzothiazol-2-yl]urea (Compound No. 6),
1-{5-[6-(4-Acetylpiperazin-1-yl)pyridin-3-yl]-4-fluoro-7-(3-fluoropyridin-2-yl)-1,3-benzothiazol-2-yl}-3-ethylurea (Compound No. 7),
1-Ethyl-3-{4-fluoro-7-(3-fluoropyridin-2-yl)-5-[6-(morpholin-4-yl)pyridin-3-yl]-1,3-benzothiazol-2-yl}urea (Compound No. 8),
1-{5-[6-(Dimethylamino)pyridin-3-yl]-4-fluoro-7-(3-fluoropyridin-2-yl)-1,3-benzothiazol-2-yl}-3-ethylurea (Compound No. 9),
1-Ethyl-3-[4-fluoro-7-(3-fluoropyridin-2-yl)-5-(6-fluoropyridin-3-yl)-1,3-benzothiazol-2-yl]urea (Compound No. 10),
1-Ethyl-3-[4-fluoro-7-(3-fluoropyridin-2-yl)-5-(2-methoxypyrimidin-5-yl)-1,3-benzothiazol-2-yl]urea (Compound No. 11),
1-[5-(6-Aminopyridin-3-yl)-4-fluoro-7-(3-fluoropyridin-2-yl)-1,3-benzothiazol-2-yl]-3-ethylurea (Compound No. 12),
1-Ethyl-3-[4-fluoro-7-(3-fluoropyridin-2-yl)-5-(pyrimidin-5-yl)-1,3-benzothiazol-2-yl]urea (Compound No. 13),
1-Ethyl-3-[4-fluoro-5-(2-fluoropyridin-4-yl)-7-(3-fluoropyridin-2-yl)-1,3-benzothiazol-2-yl]urea (Compound No. 14),
1-Ethyl-3-{4-fluoro-7-(3-fluoropyridin-2-yl)-5-[6-(piperazin-1-yl)pyridin-3-yl]-1,3-benzothiazol-2-yl}urea (Compound No. 15),
Methyl 4-{2-[(ethylcarbamoyl)amino]-4-fluoro-7-(3-fluoropyridin-2-yl)-1,3-benzothiazol-5-yl}benzoate (Compound No. 16),
Methyl 3-{2-[(ethylcarbamoyl)amino]-4-fluoro-7-(3-fluoropyridin-2-yl)-1,3-benzothiazol-5-yl}benzoate (Compound No. 17),
Methyl (4-{2-[(ethylcarbamoyl)amino]-4-fluoro-7-(3-fluoropyridin-2-yl)-1,3-benzothiazol-5-yl}phenyl)acetate (Compound No. 18),
1-{5-[2-(Cyclopentylamino)pyrimidin-5-yl]-4-fluoro-7-(3-fluoropyridin-2-yl)-1,3-benzothiazol-2-yl}-3-ethylurea (Compound No. 19),
(4-{2-[(Ethyl carbamoyl)amino]-4-fluoro-7-(3-fluoropyridin-2-yl)-1,3-benzothiazol-5-yl}phenyl)acetic acid (Compound No. 20),
4-{2-[(Ethylcarbamoyl)amino]-4-fluoro-7-(3-fluoropyridin-2-yl)-1,3-benzothiazol-5-yl}benzoic acid (Compound No. 21),
3-{2-[(Ethylcarbamoyl)amino]-4-fluoro-7-(3-fluoropyridin-2-yl)-1,3-benzothiazol-5-yl}benzoic acid (Compound No. 22),
1-Ethyl-3-{4-fluoro-7-(3-fluoropyridin-2-yl)-5-[4-(hydroxymethyl)phenyl]-1,3-benzothiazol-2-yl}urea (Compound No. 23),
1-Ethyl-3-{4-fluoro-7-(3-fluoropyridin-2-yl)-5-[3-(hydroxymethyl)phenyl]-1,3-benzothiazol-2-yl}urea (Compound No. 24),
1-Ethyl-3-[4-fluoro-7-(3-fluoropyridin-2-yl)-5-{2-[(2-methoxyethyl)amino]pyrimidin-5-yl}-1,3-benzothiazol-2-yl]urea (Compound No. 25),
1-Ethyl-3-[4-fluoro-7-(3-fluoropyridin-2-yl)-5-{6-[(2-methylpropyl)amino]pyridin-3-yl}-1,3-benzothiazol-2-yl]urea (Compound No. 26),
1-Ethyl-3-{4-fluoro-7-(3-fluoropyridin-2-yl)-5-[2-(4-hydroxypiperidin-1-yl)pyrimidin-5-yl]-1,3-benzothiazol-2-yl}urea (Compound No. 27),
1-Ethyl-3-{4-fluoro-7-(3-fluoropyridin-2-yl)-5-[2-(piperidin-1-yl)pyrimidin-5-yl]-1,3-benzothiazol-2-yl}urea (Compound No. 28),
1-Ethyl-3-{4-fluoro-7-(3-fluoropyridin-2-yl)-5-[2-(pyrrolidin-1-yl)pyrimidin-5-yl]-1,3-benzothiazol-2-yl}urea (Compound No. 29),
Ethyl 1-(5-{2-[(ethylcarbamoyl)amino]-4-fluoro-7-(3-fluoropyridin-2-yl)-1,3-benzothiazol-5-yl}pyrimidin-2-yl)piperidine-4-carboxylate (Compound No. 30),
Methyl 5-{2-[(ethylcarbamoyl)amino]-4-fluoro-7-(3-fluoropyridin-2-yl)-1,3-benzothiazol-5-yl}pyridine-2-carboxylate (Compound No. 31),
1-Ethyl-3-{4-fluoro-7-(3-fluoropyridin-2-yl)-5-[2-(4-oxopiperidin-1-yl)pyrimidin-5-yl]-1,3-benzothiazol-2-yl}urea (Compound No. 32),
Ethyl 3-[4-(5-{2-[(ethylcarbamoyl)amino]-4-fluoro-7-(3-fluoropyridin-2-yl)-1,3-benzothiazol-5-yl}pyrimidin-2-yl)piperazin-1-yl]propanoate (Compound No. 33),
1-Ethyl-3-[4-fluoro-7-(3-fluoropyridin-2-yl)-5-{2-[4-(hydroxymethyl)piperidin-1-yl]pyrimidin-5-yl}-1,3-benzothiazol-2-yl]urea (Compound No. 34),
1-Ethyl-3-[4-fluoro-7-(3-fluoropyridin-2-yl)-5-{2-[3-(hydroxymethyl)piperidin-1-yl]pyrimidin-5-yl}-1,3-benzothiazol-2-yl]urea (Compound No. 35),
Ethyl-3-[4-fluoro-7-(3-fluoropyridin-2-yl)-5-phenyl-1,3-benzothiazol-2-yl]urea (Compound No. 36),
Ethyl-3-[4-fluoro-7-(pyridin-2-yl)-5-(pyridin-3-yl)-1,3-benzothiazol-2-yl]urea (Compound No. 37),
Ethyl-3-[4-fluoro-5-(6-methylpyridin-3-yl)-7-(pyridin-2-yl)-1,3-benzothiazol-2-yl]urea (Compound No. 38),
Ethyl-3-[4-fluoro-5-(6-methoxypyridin-3-yl)-7-(pyridin-2-yl)-1,3-benzothiazol-2-yl]urea (Compound No. 39),
Ethyl-3-[4-fluoro-7-(pyridin-2-yl)-5-(pyrimidin-5-yl)-1,3-benzothiazol-2-yl]urea (Compound No. 40),
Ethyl-3-[4-fluoro-5-(1-methyl-1H-pyrazol-4-yl)-7-(pyridin-2-yl)-1,3-benzothiazol-2-yl]urea (Compound No. 41),
Methyl 4-{2-[(ethylcarbamoyl)amino]-4-fluoro-7-(pyridin-2-yl)-1,3-benzothiazol-5-yl}benzoate (Compound No. 42),
Methyl 3-{2-[(ethylcarbamoyl)amino]-4-fluoro-7-(pyridin-2-yl)-1,3-benzothiazol-5-yl}benzoate (Compound No. 43),
Ethyl (3-{2-[(ethylcarbamoyl)amino]-4-fluoro-7-(pyridin-2-yl)-1,3-benzothiazol-5-yl}phenoxy)acetate (Compound No. 44),
Ethyl (4-{2-[(ethylcarbamoyl)amino]-4-fluoro-7-(pyridin-2-yl)-1,3-benzothiazol-5-yl}phenoxy)acetate (Compound No. 45),
Methyl 4-{2-[(ethylcarbamoyl)amino]-4-fluoro-7-(pyridin-2-yl)-1,3-benzothiazol-5-yl}-2-methoxybenzoate (Compound No. 46),
Ethyl 1-[(4-{2-[(ethylcarbamoyl)amino]-4-fluoro-7-(pyridin-2-yl)-1,3-benzothiazol-5-yl}phenyl)carbonyl]piperidine-3-carboxylate (Compound No. 47),
Methyl 5-{2-[(ethylcarbamoyl)amino]-4-fluoro-7-(pyridin-2-yl)-1,3-benzothiazol-5-yl}pyridine-3-carboxylate (Compound No. 48),
Methyl (4-{2-[(ethylcarbamoyl)amino]-4-fluoro-7-(pyridin-2-yl)-1,3-benzothiazol-5-yl}-1H-pyrazol-1-yl)acetate (Compound No. 49),
Methyl 4-{2-[(ethylcarbamoyl)amino]-4-fluoro-7-(pyridin-2-yl)-1,3-benzothiazol-5-yl}thiophene-2-carboxylate (Compound No. 50),
1-Ethyl-3-[4-fluoro-5-(6-{[2-(morpholin-4-yl)ethyl]amino}pyridin-3-yl)-7-(pyridin-2-yl)-1,3-benzothiazol-2-yl]urea (Compound No. 51),
1-Ethyl-3-[4-fluoro-5-{3-[(4-methylpiperazin-1-yl)carbonyl]phenyl}-7-(pyridin-2-yl)-1,3-benzothiazol-2-yl]urea (Compound No. 52),
1-Ethyl-3-{4-fluoro-5-[6-(4-methylpiperazin-1-yl)pyridin-3-yl]-7-(pyridin-2-yl)-1,3-benzothiazol-2-yl}urea (Compound No. 53),
1-Ethyl-3-{4-fluoro-5-[2-(4-methylpiperazin-1-yl)pyridin-4-yl]-7-(pyridin-2-yl)-1,3-benzothiazol-2-yl}urea (Compound No. 54),
1-Ethyl-3-[4-fluoro-5-{4-[(4-methylpiperazin-1-yl)carbonyl]phenyl}-7-(pyridin-2-yl)-1,3-benzothiazol-2-yl]urea (Compound No. 55),
1-Ethyl-3-{4-fluoro-5-[2-(piperazin-1-yl)pyridin-3-yl]-7-(pyridin-2-yl)-1,3-benzothiazol-2-yl}urea (Compound No. 56),
1-[5-{6-[3-(Dimethylamino)propoxy]pyridin-3-yl}-4-fluoro-7-(pyridin-2-yl)-1,3-benzothiazol-2-yl]-3-ethylurea (Compound No. 57),
1-Ethyl-3-[4-fluoro-5-{2-[3-(hydroxymethyl)piperidin-1-yl]pyrimidin-5-yl}-7-(pyridin-2-yl)-1,3-benzothiazol-2-yl]urea (Compound No. 58),
1-Ethyl-3-{4-fluoro-5-[6-(morpholin-4-yl)pyridin-3-yl]-7-(pyridin-2-yl)-1,3-benzothiazol-2-yl}urea (Compound No. 59),
tert-Butyl 4-{2-[(ethylcarbamoyl)amino]-4-fluoro-7-(pyridin-2-yl)-1,3-benzothiazol-5-yl}-3,6-dihydropyridine-1(2H)-carboxylate (Compound No. 60),
Ethyl N-(5-{2-[(ethylcarbamoyl)amino]-4-fluoro-7-(pyridin-2-yl)-1,3-benzothiazol-5-yl}pyrimidin-2-yl)-N-methylglycinate (Compound No. 61),
1-{5-[2-(4-Aminopiperidin-1-yl)pyrimidin-5-yl]-4-fluoro-7-(pyridin-2-yl)-1,3-benzothiazol-2-yl}-3-ethylurea hydrochloride salt (Compound No. 62),
1-Ethyl-3-[4-fluoro-5-{2-[(piperidin-4-ylmethyl)amino]pyrimidin-5-yl}-7-(pyridin-2-yl)-1,3-benzothiazol-2-yl]urea hydrochloride salt (Compound No. 63),
Ethyl N-(5-{2-[(ethylcarbamoyl)amino]-4-fluoro-7-(3-fluoropyridin-2-yl)-1,3-benzothiazol-5-yl}pyrimidin-2-yl)glycinate (Compound No. 64),
Methyl 4-{2-[(ethylcarbamoyl)amino]-4-fluoro-7-(3-fluoropyridin-2-yl)-1,3-benzothiazol-5-yl}-2-methoxybenzoate (Compound No. 65),
Ethyl 1-[(4-{2-[(ethylcarbamoyl)amino]-4-fluoro-7-(3-fluoropyridin-2-yl)-1, 3-192 benzothiazol-5-yl}phenyl)carbonyl]piperidine-3-carboxylate (Compound No. 66),
Ethyl (4-{2-[(ethylcarbamoyl)amino]-4-fluoro-7-(3-fluoropyridin-2-yl)-1,3-benzothiazol-5-yl}phenoxy)acetate (Compound No. 67),
Ethyl (3-{2-[(ethylcarbamoyl)amino]-4-fluoro-7-(3-fluoropyridin-2-yl)-1,3-benzothiazol-5-yl}phenoxy)acetate (Compound No. 68),
Methyl 5-{2-[(ethylcarbamoyl)amino]-4-fluoro-7-(3-fluoropyridin-2-yl)-1,3-benzothiazol-5-yl}pyridine-3-carboxylate (Compound No. 69),
Ethyl N-(5-{2-[(ethylcarbamoyl)amino]-4-fluoro-7-(3-fluoropyridin-2-yl)-1,3-benzothiazol-5-yl}pyrimidin-2-yl)-N-methylglycinate (Compound No. 70),
Diethyl[(5-{2-[(ethylcarbamoyl)amino]-4-fluoro-7-(3-fluoropyridin-2-yl)-1,3-benzothiazol-5-yl}pyrimidin-2-yl)amino]propanedioate (Compound No. 71),
Dimethyl N-(5-{2-[(ethylcarbamoyl)amino]-4-fluoro-7-(3-fluoropyridin-2-yl)-1,3-benzothiazol-5-yl}pyrimidin-2-yl)-L-glutamate (Compound No. 72),
1-Ethyl-3-[4-fluoro-7-(3-fluoropyridin-2-yl)-5-{2-[(piperidin-4-ylmethyl)amino]pyrimidin-5-yl}-1,3-benzothiazol-2-yl]urea hydrochloride salt (Compound No. 73),
1-{5-[2-(3-Aminopyrrolidin-1-yl)pyrimidin-5-yl]-4-fluoro-7-(3-fluoropyridin-2-yl)-1,3-benzothiazol-2-yl}-3-ethylurea hydrochloride salt (Compound No. 74),
1-Ethyl-3-{4-fluoro-7-(3-fluoropyridin-2-yl)-5-[2-(piperazin-1-yl)pyrimidin-5-yl]-1,3-benzothiazol-2-yl}urea hydrochloride salt (Compound No. 75),
1-Ethyl-3-{4-fluoro-7-(3-fluoropyridin-2-yl)-5-[2-(piperidin-4-ylamino)pyrimidin-5-yl]-1,3-benzothiazol-2-yl}urea hydrochloride salt (Compound No. 76),
1-{5-[2-(4-Aminopiperidin-1-yl)pyrimidin-5-yl]-4-fluoro-7-(3-fluoropyridin-2-yl)-1,3-benzothiazol-2-yl}-3-ethylurea hydrochloride salt (Compound No. 77),
1-[5-{2-[2-(1,2-Dihydroxy-ethyl)-4,5-dihydroxy-tetrahydro-furan-3-yloxy]-pyrimidin-5-yl}-4-fluoro-7-(3-fluoro-pyridin-2-yl)-benzothiazol-2-yl]-3-ethyl-urea (Compound No. 78),
1-(5-{2-[5-(1,2-Dihydroxy-ethyl)-2,2-dimethyl-tetrahydro-furo[2,3-d][1,3]dioxol-6yloxy]-pyrimidine-5yl}-4-fluoro-7-pyridin-2-yl-benzpthiazol-2-yl)-3-ethyl-urea (Compound No. 79),
1-{5-[2-(2,3-Dihydroxypropoxy)pyrimidin-5-yl]-4-fluoro-7-(3-fluoropyridin-2-yl)-1,3-benzothiazol-2-yl}-3-ethylurea (Compound No. 80),
1-Ethyl-3-[4-fluoro-7-(3-fluoropyridin-2-yl)-5-{1-[2-(morpholin-4-yl)ethyl]-1H-pyrazol-4-yl}-1,3-benzothiazol-2-yl]urea hydrochloride salt (Compound No. 81),
1-Ethyl-3-{4-fluoro-5-[6-(piperazin-1-yl)pyridin-3-yl]-7-(pyridin-2-yl)-1,3-benzothiazol-2-yl}urea (Compound No. 82),
1-Ethyl-3-[4-fluoro-7-(3-fluoropyridin-2-yl)-5-{4-[(4-methylpiperazin-1-yl)carbonyl]phenyl}-1,3-benzothiazol-2-yl]urea (Compound No. 83),
1-Ethyl-3-[4-fluoro-5-(1H-pyrazol-4-yl)-7-(pyridin-2-yl)-1,3-benzothiazol-2-yl]urea Hydrochloride salt (Compound No. 84),
1-Ethyl-3-[4-fluoro-7-(pyridin-2-yl)-5-(1,2,3,6-tetrahydropyridin-4-yl)-1,3-benzothiazol-2-yl]urea Hydrochloride salt (Compound No. 85),
1-Ethyl-3-{4-fluoro-5-[2-(piperazin-1-yl)pyrimidin-5-yl]-7-(pyridin-2-yl)-1,3-benzothiazol-2-yl}urea hydrochloride salt (Compound No. 86),
1-{5-[2-(3-Aminopyrrolidin-1-yl)pyrimidin-5-yl]-4-fluoro-7-(pyridin-2-yl)-1,3-benzothiazol-2-yl}-3-ethylurea hydrochloride salt (Compound No. 87),
1-Ethyl-3-{4-fluoro-5-[2-(piperidin-4-ylamino)pyrimidin-5-yl]-7-(pyridin-2-yl)-1,3-benzothiazol-2-yl}urea hydrochloride salt (Compound No. 88),
Methyl 5-{2-[(ethylcarbamoyl)amino]-4-fluoro-7-(pyridin-2-yl)-1,3-benzothiazol-5-yl}pyridine-2-carboxylate (Compound No. 89),
Methyl (4-{2-[(ethylcarbamoyl)amino]-4-fluoro-7-(pyridin-2-yl)-1,3-benzothiazol-5-yl}phenyl)acetate (Compound No. 90),
Diethyl N-(5-{2-[(ethylcarbamoyl)amino]-4-fluoro-7-(pyridin-2-yl)-1,3-benzothiazol-5-yl}pyrimidin-2-yl)aspartate (Compound No. 91),
Diethyl N-(5-{2-[(ethylcarbamoyl)amino]-4-fluoro-7-(pyridin-2-yl)-1,3-benzothiazol-5-yl}pyrimidin-2-yl)-L-glutamate (Compound No. 92),
4-{2-[(Ethylcarbamoyl)amino]-4-fluoro-7-(pyridin-2-yl)-1,3-benzothiazol-5-yl}benzoic acid Lithium salt (Compound No. 93),
3-{2-[(Ethylcarbamoyl)amino]-4-fluoro-7-(pyridin-2-yl)-1,3-benzothiazol-5-yl}benzoic acid Lithium salt (Compound No. 94),
(3-{2-[(Ethylcarbamoyl)amino]-4-fluoro-7-(pyridin-2-yl)-1,3-benzothiazol-5-yl}phenoxy)acetic acid Lithium salt (Compound No. 95),
(4-{2-[(Ethylcarbamoyl)amino]-4-fluoro-7-(pyridin-2-yl)-1,3-benzothiazol-5-yl}phenoxy)acetic acid Lithium salt (Compound No. 96),
4-{2-[(Ethylcarbamoyl)amino]-4-fluoro-7-(pyridin-2-yl)-1,3-benzothiazol-5-yl}-2-methoxybenzoic acid (Compound No. 97),
1-[(4-{2-[(Ethylcarbamoyl)amino]-4-fluoro-7-(pyridin-2-yl)-1,3-benzothiazol-5-yl}phenyl)carbonyl]piperidine-3-carboxylic acid Lithium salt (Compound No. 98),
5-{2-[(Ethylcarbamoyl)amino]-4-fluoro-7-(pyridin-2-yl)-1,3-benzothiazol-5-yl}pyridine-3-carboxylic acid Lithium salt (Compound No. 99),
(4-{2-[(Ethylcarbamoyl)amino]-4-fluoro-7-(pyridin-2-yl)-1,3-benzothiazol-5-yl}-1H-pyrazol-1-yl)acetic acid lithium salt (Compound No. 100),
4-{2-[(Ethylcarbamoyl)amino]-4-fluoro-7-(pyridin-2-yl)-1,3-benzothiazol-5-yl}thiophene-2-carboxylic acid Lithium salt (Compound No. 101),
5-{2-[(Ethylcarbamoyl)amino]-4-fluoro-7-(pyridin-2-yl)-1,3-benzothiazol-5-yl}pyridine-2-carboxylic acid Lithium salt (Compound No. 102),
(4-{2-[(Ethylcarbamoyl)amino]-4-fluoro-7-(pyridin-2-yl)-1,3-benzothiazol-5-yl}phenyl)acetic acid Lithium salt (Compound No. 103),
1-(5-{2-[(Ethylcarbamoyl)amino]-4-fluoro-7-(pyridin-2-yl)-1,3-benzothiazol-5-yl}pyrimidin-2-yl)piperidine-4-carboxylic acid Lithium salt (Compound No. 104),
5-{2-[(1-Carboxy-2-methoxy-2-oxoethyl)amino]pyrimidin-5-yl}-2-[(ethylcarbamoyl)amino]-4-fluoro-7-(pyridin-2-yl)-1,3-benzothiazole Lithium salt (Compound No. 105),
N-(5-{2-[(Ethylcarbamoyl)amino]-4-fluoro-7-(pyridin-2-yl)-1,3-benzothiazol-5-yl}pyrimidin-2-yl)aspartic acid Lithium salt (Compound No. 106),
N-(5-{2-[(Ethylcarbamoyl)amino]-4-fluoro-7-(pyridin-2-yl)-1,3-benzothiazol-5-yl}pyrimidin-2-yl)glycine Lithium salt (Compound No. 107),
4-[(3-{2-[(Ethylcarbamoyl)amino]-4-fluoro-7-(pyridin-2-yl)-1,3-benzothiazol-5-yl}phenyl)amino]-4-oxobutanoic acid Lithium salt (Compound No. 108),
4-{2-[(Ethylcarbamoyl)amino]-4-fluoro-7-(3-fluoropyridin-2-yl)-1,3-benzothiazol-5-yl}-2-methoxybenzoic acid (Compound No. 109),
1-[(4-{2-[(Ethylcarbamoyl)amino]-4-fluoro-7-(3-fluoropyridin-2-yl)-1,3-benzothiazol-5-yl}phenyl)carbonyl]piperidine-3-carboxylic acid Lithium salt (Compound No. 110),
(4-{2-[(Ethylcarbamoyl)amino]-4-fluoro-7-(3-fluoropyridin-2-yl)-1,3-benzothiazol-5-yl}phenoxy)acetic acid Lithium salt (Compound No. 111),
5-{2-[(Ethylcarbamoyl)amino]-4-fluoro-7-(3-fluoropyridin-2-yl)-1,3-benzothiazol-5-yl}pyridine-3-carboxylic acid Lithium salt (Compound No. 112),
N-(5-{2-[(Ethylcarbamoyl)amino]-4-fluoro-7-(3-fluoropyridin-2-yl)-1,3-benzothiazol-5-yl}pyrimidin-2-yl)glycine Lithium salt (Compound No. 113),
N-(5-{2-[(Ethylcarbamoyl)amino]-4-fluoro-7-(3-fluoropyridin-2-yl)-1,3-benzothiazol-5-yl}pyrimidin-2-yl)-N-methylglycine Lithium salt (Compound No. 114),
(3-{2-[(Ethylcarbamoyl)amino]-4-fluoro-7-(3-fluoropyridin-2-yl)-1,3-benzothiazol-5-yl}phenoxy)acetic acid Lithium salt (Compound No. 115),
N-(5-{2-[(Ethylcarbamoyl)amino]-4-fluoro-7-(pyridin-2-yl)-1,3-benzothiazol-5-yl}pyrimidin-2-yl)-N-methylglycine Lithium salt (Compound No. 116),
Methyl[4-(5-{2-[(ethylcarbamoyl)amino]-4-fluoro-7-(pyridin-2-yl)-1,3-benzothiazol-5-yl}pyridin-2-yl)piperazin-1-yl]acetate hydrochloride salt (Compound No. 117),
[4-(5-{2-[(Ethylcarbamoyl)amino]-4-fluoro-7-(pyridin-2-yl)-1,3-benzothiazol-5-yl}pyrimidin-2-yl)piperazin-1-yl]acetic acid Lithium salt (Compound No. 118),
1-Ethyl-3-[4-fluoro-5-{2-[4-(2-hydroxyethyl)piperazin-1-yl]pyrimidin-5-yl}-7-(pyridin-2-yl)-1,3-benzothiazol-2-yl]urea hydrochloride salt (Compound No. 119),
3-[4-(5-{2-[(Ethylcarbamoyl)amino]-4-fluoro-7-(pyridin-2-yl)-1,3-benzothiazol-5-yl}pyrimidin-2-yl)piperazin-1-yl]propanoic acid (Compound No. 120),
3-[4-(5-{2-[(Ethylcarbamoyl)amino]-4-fluoro-7-(pyridin-2-yl)-1,3-benzothiazol-5-yl}pyrimidin-2-yl)piperazin-1-yl]propanoic acid Lithium salt (Compound No. 121),
Ethyl 3-[4-(5-{2-[(ethylcarbamoyl)amino]-4-fluoro-7-(pyridin-2-yl)-1,3-benzothiazol-5-yl}pyridin-2-yl)piperazin-1-yl]propanoate hydrochloride salt (Compound No. 122),
1-Ethyl-3-[4-fluoro-5-{4-[(4-methylpiperazin-1-yl)methyl]phenyl}-7-(pyridin-2-yl)-1,3-benzothiazol-2-yl]urea (Compound No. 123),
1-Ethyl-3-{4-fluoro-5-[4-(morpholin-4-ylmethyl)phenyl]-7-(pyridin-2-yl)-1,3-benzothiazol-2-yl}urea hydrochloride salt (Compound No. 124),
1-Ethyl-3-[4-fluoro-5-(4-{[4-(2-hydroxyethyl)piperazin-1-yl]methyl}phenyl)-7-(pyridin-2-yl)-1,3-benzothiazol-2-yl]urea hydrochloride salt (Compound No. 125),
1-Ethyl-3-[4-fluoro-5-{4-[(4-hydroxypiperidin-1-yl)methyl]phenyl}-7-(pyridin-2-yl)-1,3-benzothiazol-2-yl]urea hydrochloride salt (Compound No. 126),
1-Ethyl-3-[4-fluoro-5-(4-{[4-(hydroxymethyl)piperidin-1-yl]methyl}phenyl)-7-(pyridin-2-yl)-1,3-benzothiazol-2-yl]urea hydrochloride salt (Compound No. 127),
1-Ethyl-3-[4-fluoro-5-(5-{[4-(2-hydroxyethyl)piperazin-1-yl]methyl}thiophen-3-yl)-7-(pyridin-2-yl)-1,3-benzothiazol-2-yl]urea hydrochloride salt (Compound No. 128),
Ethyl 1-[(4-{2-[(ethylcarbamoyl)amino]-4-fluoro-7-(pyridin-2-yl)-1,3-benzothiazol-5-yl}thiophen-2-yl)methyl]piperidine-4-carboxylate hydrochloride salt (Compound No. 129),
Methyl N-[(4-{2-[(ethylcarbamoyl)amino]-4-fluoro-7-(pyridin-2-yl)-1,3-benzothiazol-5-yl}thiophen-2-yl)methyl]-1-serinate hydrochloride salt (Compound No. 130),
Ethyl N-[(4-{2-[(ethylcarbamoyl)amino]-4-fluoro-7-(pyridin-2-yl)-1,3-benzothiazol-5-yl}thiophen-2-yl)methyl]-alaninate hydrochloride salt (Compound No. 131),
1-Ethyl-3-[4-fluoro-5-{5-[(4-hydroxypiperidin-1-yl)methyl]thiophen-3-yl}-7-(pyridin-2-yl)-1,3-benzothiazol-2-yl]urea hydrochloride salt (Compound No. 132),
1-Ethyl-3-{4-fluoro-5-[5-({[2-(morpholin-4-yl)ethyl]amino}methyl)thiophen-3-yl]-7-(pyridin-2-yl)-1,3-benzothiazol-2-yl}urea hydrochloride salt (Compound No. 133),
1-[5-(5-{[bis(2-Hydroxyethyl)amino]methyl}thiophen-3-yl)-4-fluoro-7-(pyridin-2-yl)-1,3-benzothiazol-2-yl]-3-ethylurea hydrochloride salt (Compound No. 134),
1-Ethyl-3-{4-fluoro-5-[5-(morpholin-4-ylmethyl)thiophen-3-yl]-7-(pyridin-2-yl)-1,3-benzothiazol-2-yl}urea hydrochloride salt (Compound No. 135),
1-Ethyl-3-[4-fluoro-5-{5-[(4-methylpiperazin-1-yl)methyl]thiophen-3-yl}-7-(pyridin-2-yl)-1,3-benzothiazol-2-yl]urea hydrochloride salt (Compound No. 136),
1-Ethyl-3-[4-fluoro-5-(5-{[4-(2-hydroxyethyl)piperazin-1-yl]methyl}thiophen-2-yl)-7-(pyridin-2-yl)-1,3-benzothiazol-2-yl]urea hydrochloride salt (Compound No. 137) or
Ethyl 1-[(5-{2-[(ethylcarbamoyl)amino]-4-fluoro-7-(pyridin-2-yl)-1,3-benzothiazol-5-yl}thiophen-2-yl)methyl]piperidine-4-carboxylate hydrochloride salt (Compound No. 138)
or its pharmaceutically acceptable solvates, co-crystals, enantiomers, diastereomers, polymorphs.

3. A pharmaceutical composition comprising therapeutically effective amount compound of claim 1 or 2 together with one or more pharmaceutically acceptable carriers, excipients or diluents.

4. A method for treating or preventing a condition caused by or contributed to by bacterial infection comprising administering to a mammal in need thereof therapeutically effective amount of compound of claim 1-2 and pharmaceutical composition of claim 3.

5. The method of claim 4, wherein the condition is selected from community acquired pneumonia, upper or lower respiratory tract infections, skin or soft tissue infections, hospital acquired lung infections, hospital acquired bone or joint infections, mastitis, catheter infection, foreign body, prosthesis infections and peptic ulcer disease.

6. The method of claim 4, wherein the bacterial infection is caused by Gram-positive, Gram-negative or anaerobic bacteria.

7. The method of claim 6, wherein the Gram-positive, Gram-negative or anaerobic bacteria is selected from Staphylococci, Streptococci, Enterococci, Haemophilus, Moraxella spp., Chlamydia spp., Mycoplasm, Legionella spp., Mycobacterium, Helicobacter, Clostridium, Bacteroides, Corynebacterium, Bacillus and Enterobactericeae.

8. The method of claim 7, wherein the bacterium is cocci.

9. The method of claim 8, wherein the cocci is drug resistant.

10. A pharmaceutical composition of claim 3 further comprising one or more additional active ingredient selected from (i) protein synthesis inhibitors (ii) aminoglycosides (iii) cell wall synthesis inhibitors (iv) RNA and DNA synthesis inhibitors (v) fatty acid synthesis inhibitors.

11. A process for preparing a compound of Formula XVII 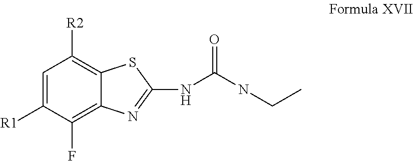
which comprises the steps of:
a) N-acylating the compound of Formula II 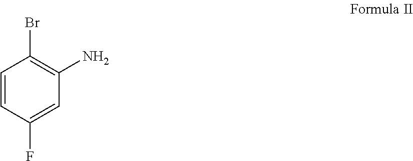
to give a compound of Formula III 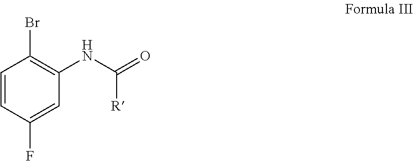
b) borylating the compound of Formula III to give a compound of Formula IV 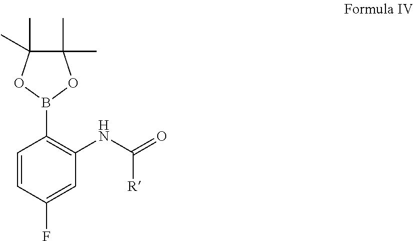
c) coupling the compound of Formula IV with a compound of Formula V
R2-Hal Formula V
to give a compound of Formula VI 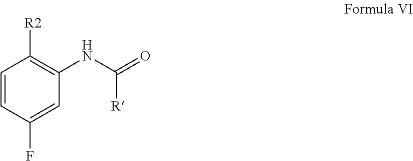
d) brominating the compound of Formula VI to give a compound of Formula VII 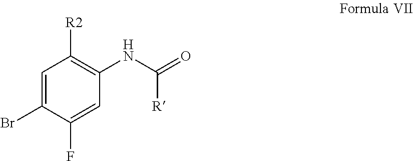
e) nitrating the compound of Formula VII to give the compound of Formula VIII 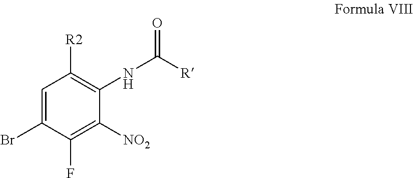
f) N-deacylating the compound of Formula VIII to give the compound of Formula IX 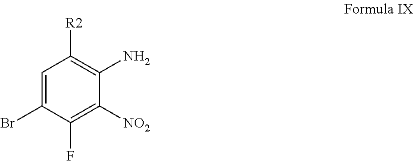
g) halogenating the compound of Formula IX to form the compound of Formula X 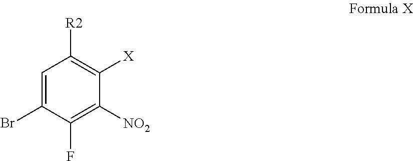
h) reducing the Compound of Formula X to give a compound of Formula XI 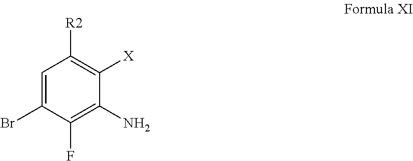
i) reacting the compound of Formula XI with benzoylisothiocyanate to give a compound of Formula XII 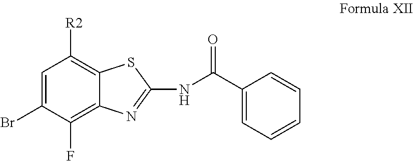
j) hydrolysing the compound of Formula XII to give a compound of Formula XIII 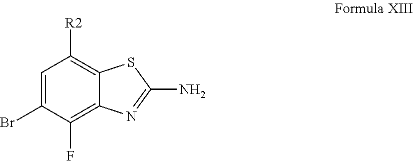
k) reacting the compound of Formula XIII with methyl(chloro)thioformate to give a compound of Formula XIV 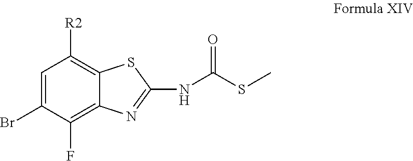
l) converting the compound of Formula XIV to a compound of Formula XV 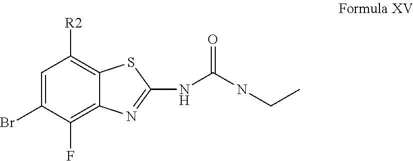
m) coupling the compound of Formula XV with a compound of Formula XVI
R1B(OH)₂ Formula XVI
to give a compound of Formula XVII 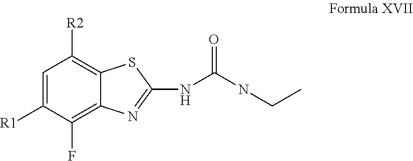
or
n) coupling the compound of Formula XV with a compound of Formula XVIa
R_{1b}B(OH)₂ Formula XVIa
to give a compound of Formula XVIII 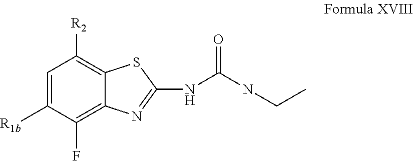
o) deprotecting the compound of Formula XVIII to give a compound of Formula XVII
wherein
R′ is alkyl, haloalkyl or aryl;
X is halogen;
Hal is halogen;
R_{1b} is cycloalkyl, aryl, heteroaryl or heterocyclyl protected with a protecting groups elected from t-butyl carbamate (t-Boc), trityl chloride, 9-fluorenyl-methyl carbamate (F-moc), allyloxycarbonyl, trifluoroacetamide or tosyl;
R₁ is cycloalkyl, aryl, heteroaryl or heterocyclyl;
R₂ is hydrogen, alkyl, cycloalkyl, aryl, aralkyl, heterocyclyl substituted alkyl, heteroaryl, heterocyclyl, —NR_{f}R_{q}, —CONR_{f}R_{q}, —COR_{f}, —SO₂R_{f}—COOR_{f}, —CR_{f}═NOR_{f} or —OCONR_{f}R_{q};
wherein R_{f} and R_{q} are independently selected from to hydrogen, alkyl, cycloalkyl, aryl, heteroaryl and heterocyclyl;
and wherein said alkyl, cycloalkyl, aryl, aralkyl, heterocyclyl substituted alkyl, heteroaryl, heterocyclyl groups have 1 to 20 carbon atoms

12. A process for preparing a compound of Formula XX 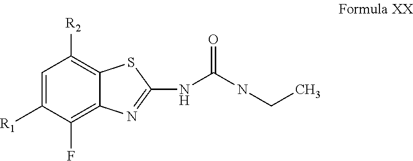
which comprise the steps of
a) hydrolysing the compound of Formula XVII (when R₁ is aryl, heteroaryl or heterocyclyl substituted with esters, alkylesters, aminoacid ester) 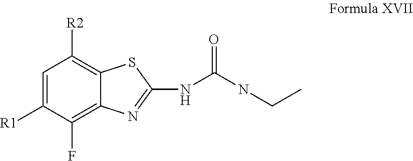
to give a compound of Formula XIX 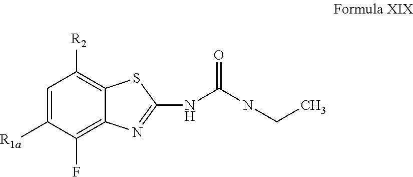
b) forming a salt of compound of Formula XIX to get a compound of Formula XX.
Wherein
R₁ₐ is aryl, heteroaryl or heterocyclyl substituted with acid, alkylacid or amino acid
R₁ is aryl, heteroaryl or heterocyclyl;
R₂ is hydrogen, alkyl, cycloalkyl, aryl, aralkyl, heterocyclyl substituted alkyl, heteroaryl, heterocyclyl, —NR_{f}R_{q}, —CONR_{f}R_{q}, —COR_{f}, —SO₂R_{f}—COOR_{f}, —CR_{f}═NOR_{f} or —OCONR_{f}R_{q};
wherein R_{f} and R_{q} are independently selected from to hydrogen, alkyl, cycloalkyl, aryl, heteroaryl and heterocyclyl;
and wherein said alkyl, cycloalkyl, aryl, aralkyl, heterocyclyl substituted alkyl, heteroaryl, heterocyclyl groups have 1 to 20 carbon atoms.

13. A process for preparing a compound of Formulae XXIII and XXV 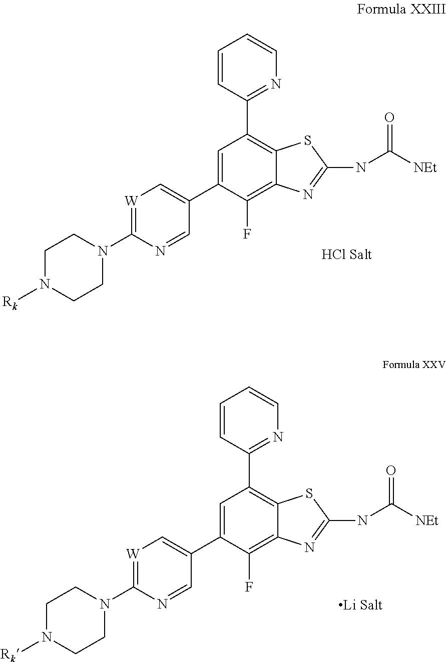
which comprise the steps of
a) coupling the compound of Formula XVII (when R₁ is pyridine or primidine substituted with piperazine wherein W is CH or N) 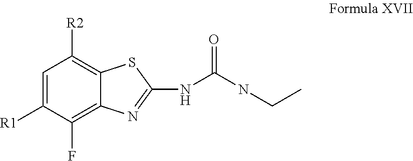
with a compound of Formula XXI
Rₖ—X Formula XXI
to give a compound of Formula XXII 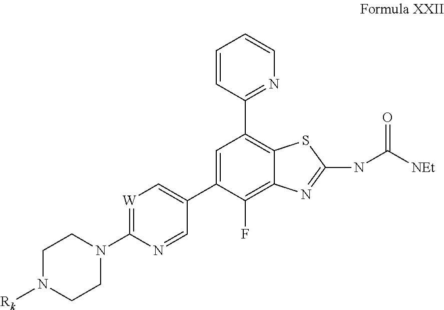
b) forming a salt of compound of Formula XXII to give a compound of Formula XXIII or
c) hydrolyzing the compound of Formula XXII to give a compound of Formula XXIV 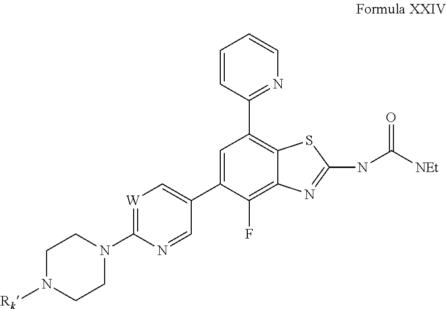
d) forming a salt of compound of Formula XXIV to give a compound of Formula XXV
wherein
Rₖ is alkyl substituted with alkoxycarbonyl;
Rₖ′ is alkyl substituted with COOH;
R₂ is hydrogen, alkyl, cycloalkyl, aryl, aralkyl, heterocyclyl substituted alkyl, heteroaryl, heterocyclyl, —NR_{f}R_{q}, —CONR_{f}R_{q}, —COR_{f}, —SO₂R_{f}—COOR_{f}, —CR_{f}═NOR_{f} or —OCONR_{f}R_{q};
wherein R_{f} and R_{q} are independently selected from to hydrogen, alkyl, cycloalkyl, aryl, heteroaryl and heterocyclyl;
and wherein said alkyl, cycloalkyl, aryl, aralkyl, heterocyclyl substituted alkyl, heteroaryl, heterocyclyl groups have 1 to 20 carbon atoms.

14. A process for preparing a compound of Formula XXVII 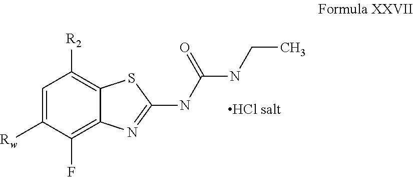
which comprise the steps of
a) reductive amination of the compound of Formula XVII (when R₁ is aryl, heteroaryl or heterocyclyl substituted with aldehyde) 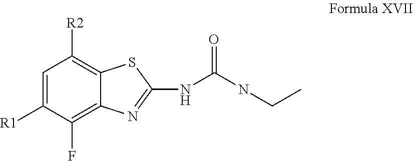
to give a compound of Formula XXVI 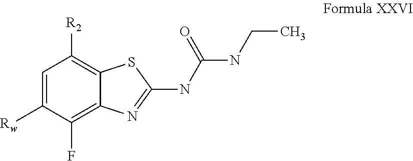
b) forming salt of compound of Formula XXVI to give a compound of Formula XXVII
wherein
R_{W} is aryl, heteroaryl or heterocyclyl substituted with CH₂-amine;
R₂ is hydrogen, alkyl, cycloalkyl, aryl, aralkyl, heterocyclyl substituted alkyl, heteroaryl, heterocyclyl, —NR_{f}R_{q}, —CONR_{f}R_{q}, —COR_{f}, —SO₂R_{f}, —COOR_{f}, —CR_{f}═NOR_{f} or —OCONR_{f}R_{q};
wherein R_{f} and R_{q} are independently selected from to hydrogen, alkyl, cycloalkyl, aryl, heteroaryl and heterocyclyl;
and wherein said alkyl, cycloalkyl, aryl, aralkyl, heterocyclyl substituted alkyl, heteroaryl, heterocyclyl groups have 1 to 20 carbon atoms
